SEQUENCE LISTING (1152 sequences)

SEQ ID NO: 1 - RNA, 21 nt, Homo sapiens
  source 1..21: mol_type = mRNA
tccagctgttgccaagacaga

SEQ ID NO: 2 - RNA, 21 nt, Homo sapiens
  source 1..21: mol_type = mRNA
cagctgttgccaagacagaga

SEQ ID NO: 3 - RNA, 21 nt, Homo sapiens
  source 1..21: mol_type = mRNA
gctgttgccaagacagagatt

SEQ ID NO: 4 - RNA, 21 nt, Homo sapiens
  source 1..21: mol_type = mRNA
ctgttgccaagacagagattg

SEQ ID NO: 5 - RNA, 21 nt, Homo sapiens
  source 1..21: mol_type = mRNA
tgttgccaagacagagattgc

SEQ ID NO: 6 - RNA, 21 nt, Homo sapiens
  source 1..21: mol_type = mRNA
gttgccaagacagagattgct

SEQ ID NO: 7 - RNA, 21 nt, Homo sapiens
  source 1..21: mol_type = mRNA
ttgccaagacagagattgctt

SEQ ID NO: 8 - RNA, 21 nt, Homo sapiens
  source 1..21: mol_type = mRNA
ccaagacagagattgctttaa

SEQ ID NO: 9 - RNA, 21 nt, Homo sapiens
  source 1..21: mol_type = mRNA
acagagattgctttaagtggc

SEQ ID NO: 10 - RNA, 21 nt, Homo sapiens
  source 1..21: mol_type = mRNA
aatcacctttattagcagcta

SEQ ID NO: 11 - RNA, 21 nt, Homo sapiens
  source 1..21: mol_type = mRNA
ggacaatattcttggtcctag

SEQ ID NO: 12 - RNA, 21 nt, Homo sapiens
  source 1..21: mol_type = mRNA
gacaatattcttggtcctaga

SEQ ID NO: 13 - RNA, 21 nt, Homo sapiens
  source 1..21: mol_type = mRNA
caatattcttggtcctagagt

SEQ ID NO: 14 - RNA, 21 nt, Homo sapiens
  source 1..21: mol_type = mRNA
atattcttggtcctagagtaa

SEQ ID NO: 15 - RNA, 21 nt, Homo sapiens
  source 1..21: mol_type = mRNA
ttcttggtcctagagtaaggc

SEQ ID NO: 16 - RNA, 21 nt, Homo sapiens
  source 1..21: mol_type = mRNA
tcttggtcctagagtaaggca

SEQ ID NO: 17 - RNA, 21 nt, Homo sapiens
  source 1..21: mol_type = mRNA
cttggtcctagagtaaggcac

SEQ ID NO: 18 - RNA, 21 nt, Homo sapiens
  source 1..21: mol_type = mRNA
gcacatttgggctccaaagac

SEQ ID NO: 19 - RNA, 21 nt, Homo sapiens
  source 1..21: mol_type = mRNA
cacatttgggctccaaagaca

SEQ ID NO: 20 - RNA, 21 nt, Homo sapiens
  source 1..21: mol_type = mRNA
acatttgggctccaaagacag

SEQ ID NO: 21 - RNA, 21 nt, Homo sapiens
  source 1..21: mol_type = mRNA
catttgggctccaaagacaga

SEQ ID NO: 22 - RNA, 21 nt, Homo sapiens
  source 1..21: mol_type = mRNA
agaaataacttttcttgccaa

SEQ ID NO: 23 - RNA, 21 nt, Homo sapiens
  source 1..21: mol_type = mRNA
aataacttttcttgccaacca

SEQ ID NO: 24 - RNA, 21 nt, Homo sapiens
  source 1..21: mol_type = mRNA
ataacttttcttgccaaccac

SEQ ID NO: 25 - RNA, 21 nt, Homo sapiens
  source 1..21: mol_type = mRNA
taacttttcttgccaaccaca

SEQ ID NO: 26 - RNA, 21 nt, Homo sapiens
  source 1..21: mol_type = mRNA
ttttcttgccaaccacactct

SEQ ID NO: 27 - RNA, 21 nt, Homo sapiens
  source 1..21: mol_type = mRNA
tttcttgccaaccacactcta

SEQ ID NO: 28 - RNA, 21 nt, Homo sapiens
  source 1..21: mol_type = mRNA
ttcttgccaaccacactctaa

SEQ ID NO: 29 - RNA, 21 nt, Homo sapiens
  source 1..21: mol_type = mRNA
tcttgccaaccacactctaaa

SEQ ID NO: 30 - RNA, 21 nt, Homo sapiens
  source 1..21: mol_type = mRNA
cttgccaaccacactctaaat

SEQ ID NO: 31 - RNA, 21 nt, Homo sapiens
  source 1..21: mol_type = mRNA
ttgccaaccacactctaaatg

SEQ ID NO: 32 - RNA, 21 nt, Homo sapiens
  source 1..21: mol_type = mRNA
tgccaaccacactctaaatgg

SEQ ID NO: 33 - RNA, 21 nt, Homo sapiens
  source 1..21: mol_type = mRNA
caaccacactctaaatggaga

SEQ ID NO: 34 - RNA, 21 nt, Homo sapiens
  source 1..21: mol_type = mRNA
aatccttcgaaatgcagagag

SEQ ID NO: 35 - RNA, 21 nt, Homo sapiens
  source 1..21: mol_type = mRNA
ccttcgaaatgcagagagtgg

SEQ ID NO: 36 - RNA, 21 nt, Homo sapiens
  source 1..21: mol_type = mRNA
agagtggtgctatagatgtaa

SEQ ID NO: 37 - RNA, 21 nt, Homo sapiens
  source 1..21: mol_type = mRNA
gagtggtgctatagatgtaaa

SEQ ID NO: 38 - RNA, 21 nt, Homo sapiens
  source 1..21: mol_type = mRNA
tggtgctatagatgtaaagtt

SEQ ID NO: 39 - RNA, 21 nt, Homo sapiens
  source 1..21: mol_type = mRNA
gtgctatagatgtaaagtttt

SEQ ID NO: 40 - RNA, 21 nt, Homo sapiens
  source 1..21: mol_type = mRNA
tttgtcttgtctgaaaaggga

SEQ ID NO: 41 - RNA, 21 nt, Homo sapiens
  source 1..21: mol_type = mRNA
tcttgtctgaaaagggagtga

SEQ ID NO: 42 - RNA, 21 nt, Homo sapiens
  source 1..21: mol_type = mRNA
gtctgaaaagggagtgattat

SEQ ID NO: 43 - RNA, 21 nt, Homo sapiens
  source 1..21: mol_type = mRNA
tctgaaaagggagtgattatt

SEQ ID NO: 44 - RNA, 21 nt, Homo sapiens
  source 1..21: mol_type = mRNA
aaaagggagtgattattgttt

SEQ ID NO: 45 - RNA, 21 nt, Homo sapiens
  source 1..21: mol_type = mRNA
atctttgatggaaactggaat

SEQ ID NO: 46 - RNA, 21 nt, Homo sapiens
  source 1..21: mol_type = mRNA
attatacttccacagacagaa

SEQ ID NO: 47 - RNA, 21 nt, Homo sapiens
  source 1..21: mol_type = mRNA
ttatacttccacagacagaac

SEQ ID NO: 48 - RNA, 21 nt, Homo sapiens
  source 1..21: mol_type = mRNA
tacttccacagacagaactta

SEQ ID NO: 49 - RNA, 21 nt, Homo sapiens
  source 1..21: mol_type = mRNA
cacagacagaacttagtttct

SEQ ID NO: 50 - RNA, 21 nt, Homo sapiens
  source 1..21: mol_type = mRNA
tagtttctacctcccacttca

SEQ ID NO: 51 - RNA, 21 nt, Homo sapiens
  source 1..21: mol_type = mRNA
agtttctacctcccacttcat

SEQ ID NO: 52 - RNA, 21 nt, Homo sapiens
  source 1..21: mol_type = mRNA
cacatataatccggaaaggaa

SEQ ID NO: 53 - RNA, 21 nt, Homo sapiens
  source 1..21: mol_type = mRNA
tataatccggaaaggaagaat

SEQ ID NO: 54 - RNA, 21 nt, Homo sapiens
  source 1..21: mol_type = mRNA
aatccggaaaggaagaatatg

SEQ ID NO: 55 - RNA, 21 nt, Homo sapiens
  source 1..21: mol_type = mRNA
tccggaaaggaagaatatgga

SEQ ID NO: 56 - RNA, 21 nt, Homo sapiens
  source 1..21: mol_type = mRNA
aaggaagaatatggatgcata

SEQ ID NO: 57 - RNA, 21 nt, Homo sapiens
  source 1..21: mol_type = mRNA
gaagaatatggatgcataagg

SEQ ID NO: 58 - RNA, 21 nt, Homo sapiens
  source 1..21: mol_type = mRNA
agaatatggatgcataaggaa

SEQ ID NO: 59 - RNA, 21 nt, Homo sapiens
  source 1..21: mol_type = mRNA
gaatatggatgcataaggaaa

SEQ ID NO: 60 - RNA, 21 nt, Homo sapiens
  source 1..21: mol_type = mRNA
aatatggatgcataaggaaag

SEQ ID NO: 61 - RNA, 21 nt, Homo sapiens
  source 1..21: mol_type = mRNA
atggatgcataaggaaagaca

SEQ ID NO: 62 - RNA, 21 nt, Homo sapiens
  source 1..21: mol_type = mRNA
tggatgcataaggaaagacaa

SEQ ID NO: 63 - RNA, 21 nt, Homo sapiens
  source 1..21: mol_type = mRNA
ggatgcataaggaaagacaag

SEQ ID NO: 64 - RNA, 21 nt, Homo sapiens
  source 1..21: mol_type = mRNA
aggaaagacaagaaaatgtcc

SEQ ID NO: 65 - RNA, 21 nt, Homo sapiens
  source 1..21: mol_type = mRNA
aagacaagaaaatgtccagaa

SEQ ID NO: 66 - RNA, 21 nt, Homo sapiens
  source 1..21: mol_type = mRNA
tatcttagaaggcacagagag

SEQ ID NO: 67 - RNA, 21 nt, Homo sapiens
  source 1..21: mol_type = mRNA
agagagaatggaagatcaggg

SEQ ID NO: 68 - RNA, 21 nt, Homo sapiens
  source 1..21: mol_type = mRNA
gagaatggaagatcagggtca

SEQ ID NO: 69 - RNA, 21 nt, Homo sapiens
  source 1..21: mol_type = mRNA
agaatggaagatcagggtcag

SEQ ID NO: 70 - RNA, 21 nt, Homo sapiens
  source 1..21: mol_type = mRNA
gaatggaagatcagggtcaga

SEQ ID NO: 71 - RNA, 21 nt, Homo sapiens
  source 1..21: mol_type = mRNA
aatggaagatcagggtcagag

SEQ ID NO: 72 - RNA, 21 nt, Homo sapiens
  source 1..21: mol_type = mRNA
atggaagatcagggtcagagt

SEQ ID NO: 73 - RNA, 21 nt, Homo sapiens
  source 1..21: mol_type = mRNA
tggaagatcagggtcagagta

SEQ ID NO: 74 - RNA, 21 nt, Homo sapiens
  source 1..21: mol_type = mRNA
ggaagatcagggtcagagtat

SEQ ID NO: 75 - RNA, 21 nt, Homo sapiens
  source 1..21: mol_type = mRNA
gaagatcagggtcagagtatt

SEQ ID NO: 76 - RNA, 21 nt, Homo sapiens
  source 1..21: mol_type = mRNA
gatcagggtcagagtattatt

SEQ ID NO: 77 - RNA, 21 nt, Homo sapiens
  source 1..21: mol_type = mRNA
cagggtcagagtattattcca

SEQ ID NO: 78 - RNA, 21 nt, Homo sapiens
  source 1..21: mol_type = mRNA
tggagaagtgattcctgtaat

SEQ ID NO: 79 - RNA, 21 nt, Homo sapiens
  source 1..21: mol_type = mRNA
ctgtaatggaactgctttcat

SEQ ID NO: 80 - RNA, 21 nt, Homo sapiens
  source 1..21: mol_type = mRNA
atggaactgctttcatctatg

SEQ ID NO: 81 - RNA, 21 nt, Homo sapiens
  source 1..21: mol_type = mRNA
ttcatctatgaaatcacacag

SEQ ID NO: 82 - RNA, 21 nt, Homo sapiens
  source 1..21: mol_type = mRNA
ctatgaaatcacacagtgttc

SEQ ID NO: 83 - RNA, 21 nt, Homo sapiens
  source 1..21: mol_type = mRNA
tcctgaagaaatagatatagc

SEQ ID NO: 84 - RNA, 21 nt, Homo sapiens
  source 1..21: mol_type = mRNA
aatagatatagctgatacagt

SEQ ID NO: 85 - RNA, 21 nt, Homo sapiens
  source 1..21: mol_type = mRNA
ctgatacagtactcaatgatg

SEQ ID NO: 86 - RNA, 21 nt, Homo sapiens
  source 1..21: mol_type = mRNA
tgaaggctttcttctcaatgc

SEQ ID NO: 87 - RNA, 21 nt, Homo sapiens
  source 1..21: mol_type = mRNA
gaaggctttcttctcaatgcc

SEQ ID NO: 88 - RNA, 21 nt, Homo sapiens
  source 1..21: mol_type = mRNA
aaggctttcttctcaatgcca

SEQ ID NO: 89 - RNA, 21 nt, Homo sapiens
  source 1..21: mol_type = mRNA
cttctcaatgccatcagctca

SEQ ID NO: 90 - RNA, 21 nt, Homo sapiens
  source 1..21: mol_type = mRNA
ttctcaatgccatcagctcac

SEQ ID NO: 91 - RNA, 21 nt, Homo sapiens
  source 1..21: mol_type = mRNA
tctcaatgccatcagctcaca

SEQ ID NO: 92 - RNA, 21 nt, Homo sapiens
  source 1..21: mol_type = mRNA
tcaatgccatcagctcacact

SEQ ID NO: 93 - RNA, 21 nt, Homo sapiens
  source 1..21: mol_type = mRNA
caaacctgtggctgttccgtt

SEQ ID NO: 94 - RNA, 21 nt, Homo sapiens
  source 1..21: mol_type = mRNA
acctgtggctgttccgttgta

SEQ ID NO: 95 - RNA, 21 nt, Homo sapiens
  source 1..21: mol_type = mRNA
ctgtggctgttccgttgtagt

SEQ ID NO: 96 - RNA, 21 nt, Homo sapiens
  source 1..21: mol_type = mRNA
gttgtagtaggtagcagtgca

SEQ ID NO: 97 - RNA, 21 nt, Homo sapiens
  source 1..21: mol_type = mRNA
ggtagcagtgcagagaaagta

SEQ ID NO: 98 - RNA, 21 nt, Homo sapiens
  source 1..21: mol_type = mRNA
agcagtgcagagaaagtaaat

SEQ ID NO: 99 - RNA, 21 nt, Homo sapiens
  source 1..21: mol_type = mRNA
gcagtgcagagaaagtaaata

SEQ ID NO: 100 - RNA, 21 nt, Homo sapiens
  source 1..21: mol_type = mRNA
gcagagaaagtaaataagata

SEQ ID NO: 101 - RNA, 21 nt, Homo sapiens
  source 1..21: mol_type = mRNA
gatagtcagaacattatgcct

SEQ ID NO: 102 - RNA, 21 nt, Homo sapiens
  source 1..21: mol_type = mRNA
tgaagcagaatcatcatttaa

SEQ ID NO: 103 - RNA, 21 nt, Homo sapiens
  source 1..21: mol_type = mRNA
gaagcagaatcatcatttaaa

SEQ ID NO: 104 - RNA, 21 nt, Homo sapiens
  source 1..21: mol_type = mRNA
ctgctaaaggattcaactgga

SEQ ID NO: 105 - RNA, 21 nt, Homo sapiens
  source 1..21: mol_type = mRNA
ttccggcaagtcatgtatgct

SEQ ID NO: 106 - RNA, 21 nt, Homo sapiens
  source 1..21: mol_type = mRNA
ctccatatcccaccacacaca

SEQ ID NO: 107 - RNA, 21 nt, Homo sapiens
  source 1..21: mol_type = mRNA
tgccaccctgtcatgaacata

SEQ ID NO: 108 - RNA, 21 nt, Homo sapiens
  source 1..21: mol_type = mRNA
gccaccctgtcatgaacatat

SEQ ID NO: 109 - RNA, 21 nt, Homo sapiens
  source 1..21: mol_type = mRNA
ccctgtcatgaacatatttat

SEQ ID NO: 110 - RNA, 21 nt, Homo sapiens
  source 1..21: mol_type = mRNA
ttataatcagcgtagatacat

SEQ ID NO: 111 - RNA, 21 nt, Homo sapiens
  source 1..21: mol_type = mRNA
aatcagcgtagatacatgaga

SEQ ID NO: 112 - RNA, 21 nt, Homo sapiens
  source 1..21: mol_type = mRNA
atggctcaggatacgatcatc

SEQ ID NO: 113 - RNA, 21 nt, Homo sapiens
  source 1..21: mol_type = mRNA
cgatcatctacactgacgaaa

SEQ ID NO: 114 - RNA, 21 nt, Homo sapiens
  source 1..21: mol_type = mRNA
tgacgaaagctttactcctga

SEQ ID NO: 115 - RNA, 21 nt, Homo sapiens
  source 1..21: mol_type = mRNA
ctttactcctgatttgaatat

SEQ ID NO: 116 - RNA, 21 nt, Homo sapiens
  source 1..21: mol_type = mRNA
tttcaagatgtcttacacaga

SEQ ID NO: 117 - RNA, 21 nt, Homo sapiens
  source 1..21: mol_type = mRNA
atgtcttacacagagacactc

SEQ ID NO: 118 - RNA, 21 nt, Homo sapiens
  source 1..21: mol_type = mRNA
tgtcttacacagagacactct

SEQ ID NO: 119 - RNA, 21 nt, Homo sapiens
  source 1..21: mol_type = mRNA
gtcttacacagagacactcta

SEQ ID NO: 120 - RNA, 21 nt, Homo sapiens
  source 1..21: mol_type = mRNA
tcttacacagagacactctag

SEQ ID NO: 121 - RNA, 21 nt, Homo sapiens
  source 1..21: mol_type = mRNA
cttacacagagacactctagt

SEQ ID NO: 122 - RNA, 21 nt, Homo sapiens
  source 1..21: mol_type = mRNA
ttacacagagacactctagtg

SEQ ID NO: 123 - RNA, 21 nt, Homo sapiens
  source 1..21: mol_type = mRNA
tacacagagacactctagtga

SEQ ID NO: 124 - RNA, 21 nt, Homo sapiens
  source 1..21: mol_type = mRNA
acacagagacactctagtgaa

SEQ ID NO: 125 - RNA, 21 nt, Homo sapiens
  source 1..21: mol_type = mRNA
cacagagacactctagtgaaa

SEQ ID NO: 126 - RNA, 21 nt, Homo sapiens
  source 1..21: mol_type = mRNA
acagagacactctagtgaaag

SEQ ID NO: 127 - RNA, 21 nt, Homo sapiens
  source 1..21: mol_type = mRNA
cagaagtactttccttgcaca

SEQ ID NO: 128 - RNA, 21 nt, Homo sapiens
  source 1..21: mol_type = mRNA
agtactttccttgcacagttt

SEQ ID NO: 129 - RNA, 21 nt, Homo sapiens
  source 1..21: mol_type = mRNA
ccttcacagaaaagccttgac

SEQ ID NO: 130 - RNA, 21 nt, Homo sapiens
  source 1..21: mol_type = mRNA
cacagaaaagccttgacacta

SEQ ID NO: 131 - RNA, 21 nt, Homo sapiens
  source 1..21: mol_type = mRNA
acagaaaagccttgacactaa

SEQ ID NO: 132 - RNA, 21 nt, Homo sapiens
  source 1..21: mol_type = mRNA
cagaaaagccttgacactaat

SEQ ID NO: 133 - RNA, 21 nt, Homo sapiens
  source 1..21: mol_type = mRNA
acgatacgcagaagggaaaaa

SEQ ID NO: 134 - RNA, 21 nt, Homo sapiens
  source 1..21: mol_type = mRNA
cgatacgcagaagggaaaaaa

SEQ ID NO: 135 - RNA, 21 nt, Homo sapiens
  source 1..21: mol_type = mRNA
gaccttgatttaacagcagag

SEQ ID NO: 136 - RNA, 21 nt, Homo sapiens
  source 1..21: mol_type = mRNA
accttgatttaacagcagagg

SEQ ID NO: 137 - RNA, 21 nt, Homo sapiens
  source 1..21: mol_type = mRNA
ccttgatttaacagcagaggg

SEQ ID NO: 138 - RNA, 21 nt, Homo sapiens
  source 1..21: mol_type = mRNA
cttgatttaacagcagagggc

SEQ ID NO: 139 - RNA, 21 nt, Homo sapiens
  source 1..21: mol_type = mRNA
ttgatttaacagcagagggcg

SEQ ID NO: 140 - RNA, 21 nt, Homo sapiens
  source 1..21: mol_type = mRNA
tgatttaacagcagagggcga

SEQ ID NO: 141 - RNA, 21 nt, Homo sapiens
  source 1..21: mol_type = mRNA
atttaacagcagagggcgatc

SEQ ID NO: 142 - RNA, 21 nt, Homo sapiens
  source 1..21: mol_type = mRNA
tttaacagcagagggcgatct

SEQ ID NO: 143 - RNA, 21 nt, Homo sapiens
  source 1..21: mol_type = mRNA
taacagcagagggcgatctta

SEQ ID NO: 144 - RNA, 21 nt, Homo sapiens
  source 1..21: mol_type = mRNA
aacagcagagggcgatcttaa

SEQ ID NO: 145 - RNA, 21 nt, Homo sapiens
  source 1..21: mol_type = mRNA
cagcagagggcgatcttaaca

SEQ ID NO: 146 - RNA, 21 nt, Homo sapiens
  source 1..21: mol_type = mRNA
agagggcgatcttaacataat

SEQ ID NO: 147 - RNA, 21 nt, Homo sapiens
  source 1..21: mol_type = mRNA
gagggcgatcttaacataata

SEQ ID NO: 148 - RNA, 21 nt, Homo sapiens
  source 1..21: mol_type = mRNA
agggcgatcttaacataataa

SEQ ID NO: 149 - RNA, 21 nt, Homo sapiens
  source 1..21: mol_type = mRNA
gggcgatcttaacataataat

SEQ ID NO: 150 - RNA, 21 nt, Homo sapiens
  source 1..21: mol_type = mRNA
cgatcttaacataataatggc

SEQ ID NO: 151 - RNA, 21 nt, Homo sapiens
  source 1..21: mol_type = mRNA
gatcttaacataataatggct

SEQ ID NO: 152 - RNA, 21 nt, Homo sapiens
  source 1..21: mol_type = mRNA
tcttaacataataatggctct

SEQ ID NO: 153 - RNA, 21 nt, Homo sapiens
  source 1..21: mol_type = mRNA
cttaacataataatggctctg

SEQ ID NO: 154 - RNA, 21 nt, Homo sapiens
  source 1..21: mol_type = mRNA
ggctctggctgagaaaattaa

SEQ ID NO: 155 - RNA, 21 nt, Homo sapiens
  source 1..21: mol_type = mRNA
ctctggctgagaaaattaaac

SEQ ID NO: 156 - RNA, 21 nt, Homo sapiens
  source 1..21: mol_type = mRNA
taaaccaggcctacactcttt

SEQ ID NO: 157 - RNA, 21 nt, Homo sapiens
  source 1..21: mol_type = mRNA
tggaagacctttctacactag

SEQ ID NO: 158 - RNA, 21 nt, Homo sapiens
  source 1..21: mol_type = mRNA
ggaagacctttctacactagt

SEQ ID NO: 159 - RNA, 21 nt, Homo sapiens
  source 1..21: mol_type = mRNA
gaagacctttctacactagtg

SEQ ID NO: 160 - RNA, 21 nt, Homo sapiens
  source 1..21: mol_type = mRNA
aagacctttctacactagtgt

SEQ ID NO: 161 - RNA, 21 nt, Homo sapiens
  source 1..21: mol_type = mRNA
tgcaagaacgagatgttctaa

SEQ ID NO: 162 - RNA, 21 nt, Homo sapiens
  source 1..21: mol_type = mRNA
gtgtaacttaataagcctatt

SEQ ID NO: 163 - RNA, 21 nt, Homo sapiens
  source 1..21: mol_type = mRNA
taacttaataagcctattcca

SEQ ID NO: 164 - RNA, 21 nt, Homo sapiens
  source 1..21: mol_type = mRNA
taataagcctattccatcaca

SEQ ID NO: 165 - RNA, 21 nt, Homo sapiens
  source 1..21: mol_type = mRNA
cagcaattgcagttaagtaag

SEQ ID NO: 166 - RNA, 21 nt, Homo sapiens
  source 1..21: mol_type = mRNA
tgcagttaagtaagttacact

SEQ ID NO: 167 - RNA, 21 nt, Homo sapiens
  source 1..21: mol_type = mRNA
gtaagttacactacagttctc

SEQ ID NO: 168 - RNA, 21 nt, Homo sapiens
  source 1..21: mol_type = mRNA
caggtgcatcattacattggg

SEQ ID NO: 169 - RNA, 21 nt, Homo sapiens
  source 1..21: mol_type = mRNA
tgcttttgggatacagaccta

SEQ ID NO: 170 - RNA, 21 nt, Homo sapiens
  source 1..21: mol_type = mRNA
gcttttgggatacagacctat

SEQ ID NO: 171 - RNA, 21 nt, Homo sapiens
  source 1..21: mol_type = mRNA
ttttgggatacagacctatgt

SEQ ID NO: 172 - RNA, 21 nt, Homo sapiens
  source 1..21: mol_type = mRNA
ttgggatacagacctatgttt

SEQ ID NO: 173 - RNA, 21 nt, Homo sapiens
  source 1..21: mol_type = mRNA
tgggatacagacctatgttta

SEQ ID NO: 174 - RNA, 21 nt, Homo sapiens
  source 1..21: mol_type = mRNA
tacagacctatgtttacaata

SEQ ID NO: 175 - RNA, 21 nt, Homo sapiens
  source 1..21: mol_type = mRNA
cagacctatgtttacaatata

SEQ ID NO: 176 - RNA, 21 nt, Homo sapiens
  source 1..21: mol_type = mRNA
atacatgattcatggtttaca

SEQ ID NO: 177 - RNA, 21 nt, Homo sapiens
  source 1..21: mol_type = mRNA
gttgactttctatcttttggc

SEQ ID NO: 178 - RNA, 21 nt, Homo sapiens
  source 1..21: mol_type = mRNA
attccacagaaagaaagtgag

SEQ ID NO: 179 - RNA, 21 nt, Homo sapiens
  source 1..21: mol_type = mRNA
tgatcaagcagatgtttaatt

SEQ ID NO: 180 - RNA, 21 nt, Homo sapiens
  source 1..21: mol_type = mRNA
caagcagatgtttaattggaa

SEQ ID NO: 181 - RNA, 21 nt, Homo sapiens
  source 1..21: mol_type = mRNA
ccctgggattcagtctgtaga

SEQ ID NO: 182 - RNA, 21 nt, Homo sapiens
  source 1..21: mol_type = mRNA
ttcagtctgtagaaatgtcta

SEQ ID NO: 183 - RNA, 21 nt, Homo sapiens
  source 1..21: mol_type = mRNA
tcctggtgaaccacagttagg

SEQ ID NO: 184 - RNA, 21 nt, Homo sapiens
  source 1..21: mol_type = mRNA
tatgtagttgagctctgtaaa

SEQ ID NO: 185 - RNA, 21 nt, Homo sapiens
  source 1..21: mol_type = mRNA
atgtagttgagctctgtaaaa

SEQ ID NO: 186 - RNA, 21 nt, Homo sapiens
  source 1..21: mol_type = mRNA
gtagttgagctctgtaaaagg

SEQ ID NO: 187 - RNA, 21 nt, Homo sapiens
  source 1..21: mol_type = mRNA
agttgagctctgtaaaaggaa

SEQ ID NO: 188 - RNA, 21 nt, Homo sapiens
  source 1..21: mol_type = mRNA
gttgagctctgtaaaaggaaa

SEQ ID NO: 189 - RNA, 21 nt, Homo sapiens
  source 1..21: mol_type = mRNA
ttgagccaaattgaaatgtgc

SEQ ID NO: 190 - RNA, 21 nt, Homo sapiens
  source 1..21: mol_type = mRNA
tgtgcacctcctgtgcctttt

SEQ ID NO: 191 - RNA, 21 nt, Homo sapiens
  source 1..21: mol_type = mRNA
ttcagatttcactggtcagtc

SEQ ID NO: 192 - RNA, 21 nt, Homo sapiens
  source 1..21: mol_type = mRNA
tcttaccatgtacctgctttg

SEQ ID NO: 193 - RNA, 21 nt, Homo sapiens
  source 1..21: mol_type = mRNA
gtcccgctaggaaagagaggt

SEQ ID NO: 194 - RNA, 21 nt, Homo sapiens
  source 1..21: mol_type = mRNA
gtcaaacagcgacaagttccg

SEQ ID NO: 195 - RNA, 21 nt, Homo sapiens
  source 1..21: mol_type = mRNA
tcaaacagcgacaagttccgc

SEQ ID NO: 196 - RNA, 21 nt, Homo sapiens
  source 1..21: mol_type = mRNA
cagcgacaagttccgcccacg

SEQ ID NO: 197 - RNA, 21 nt, Homo sapiens
  source 1..21: mol_type = mRNA
cgacaagttccgcccacgtaa

SEQ ID NO: 198 - RNA, 21 nt, Homo sapiens
  source 1..21: mol_type = mRNA
gacaagttccgcccacgtaaa

SEQ ID NO: 199 - RNA, 21 nt, Homo sapiens
  source 1..21: mol_type = mRNA
aagttccgcccacgtaaaaga

SEQ ID NO: 200 - RNA, 21 nt, Homo sapiens
  source 1..21: mol_type = mRNA
ttccgcccacgtaaaagatga

SEQ ID NO: 201 - RNA, 21 nt, Homo sapiens
  source 1..21: mol_type = mRNA
tccgcccacgtaaaagatgac

SEQ ID NO: 202 - RNA, 21 nt, Homo sapiens
  source 1..21: mol_type = mRNA
ccgcccacgtaaaagatgacg

SEQ ID NO: 203 - RNA, 21 nt, Homo sapiens
  source 1..21: mol_type = mRNA
cgcccacgtaaaagatgacgc

SEQ ID NO: 204 - RNA, 21 nt, Homo sapiens
  source 1..21: mol_type = mRNA
ccacgtaaaagatgacgcttg

SEQ ID NO: 205 - RNA, 21 nt, Homo sapiens
  source 1..21: mol_type = mRNA
gtgtgtcagccgtccctgctg

SEQ ID NO: 206 - RNA, 21 nt, Homo sapiens
  source 1..21: mol_type = mRNA
tgctgcccggttgcttctctt

SEQ ID NO: 207 - RNA, 21 nt, Homo sapiens
  source 1..21: mol_type = mRNA
gtctagcaagagcaggtgtgg

SEQ ID NO: 208 - RNA, 21 nt, Homo sapiens
  source 1..21: mol_type = mRNA
caagagcaggtgtgggtttag

SEQ ID NO: 209 - RNA, 21 nt, Homo sapiens
  source 1..21: mol_type = mRNA
aggtgtgggtttaggaggtgt

SEQ ID NO: 210 - RNA, 21 nt, Homo sapiens
  source 1..21: mol_type = mRNA
ggtgtgggtttaggaggtgtg

SEQ ID NO: 211 - RNA, 21 nt, Homo sapiens
  source 1..21: mol_type = mRNA
gtgtgggtttaggaggtgtgt

SEQ ID NO: 212 - RNA, 21 nt, Homo sapiens
  source 1..21: mol_type = mRNA
gtgggtttaggaggtgtgtgt

SEQ ID NO: 213 - RNA, 21 nt, Homo sapiens
  source 1..21: mol_type = mRNA
tgggtttaggaggtgtgtgtt

SEQ ID NO: 214 - RNA, 21 nt, Homo sapiens
  source 1..21: mol_type = mRNA
gggtttaggaggtgtgtgttt

SEQ ID NO: 215 - RNA, 21 nt, Homo sapiens
  source 1..21: mol_type = mRNA
gtttaggaggtgtgtgttttt

SEQ ID NO: 216 - RNA, 21 nt, Homo sapiens
  source 1..21: mol_type = mRNA
tttaggaggtgtgtgtttttg

SEQ ID NO: 217 - RNA, 21 nt, Homo sapiens
  source 1..21: mol_type = mRNA
ttaggaggtgtgtgtttttgt

SEQ ID NO: 218 - RNA, 21 nt, Homo sapiens
  source 1..21: mol_type = mRNA
gtttttgtttttcccaccctc

SEQ ID NO: 219 - RNA, 21 nt, Homo sapiens
  source 1..21: mol_type = mRNA
tcgctgagggtgaacaagaaa

SEQ ID NO: 220 - RNA, 21 nt, Homo sapiens
  source 1..21: mol_type = mRNA
cgctgagggtgaacaagaaaa

SEQ ID NO: 221 - RNA, 21 nt, Homo sapiens
  source 1..21: mol_type = mRNA
gagggtgaacaagaaaagacc

SEQ ID NO: 222 - RNA, 21 nt, Homo sapiens
  source 1..21: mol_type = mRNA
ggtgaacaagaaaagacctga

SEQ ID NO: 223 - RNA, 21 nt, Homo sapiens
  source 1..21: mol_type = mRNA
gtgaacaagaaaagacctgat

SEQ ID NO: 224 - RNA, 21 nt, Homo sapiens
  source 1..21: mol_type = mRNA
tgaacaagaaaagacctgata

SEQ ID NO: 225 - RNA, 21 nt, Homo sapiens
  source 1..21: mol_type = mRNA
aacaagaaaagacctgataaa

SEQ ID NO: 226 - RNA, 21 nt, Homo sapiens
  source 1..21: mol_type = mRNA
agacctgataaagattaacca

SEQ ID NO: 227 - RNA, 21 nt, Homo sapiens
  source 1..21: mol_type = mRNA
gacctgataaagattaaccag

SEQ ID NO: 228 - RNA, 21 nt, Homo sapiens
  source 1..21: mol_type = mRNA
acctgataaagattaaccaga

SEQ ID NO: 229 - RNA, 21 nt, Homo sapiens
  source 1..21: mol_type = mRNA
ctgataaagattaaccagaag

SEQ ID NO: 230 - RNA, 21 nt, Homo sapiens
  source 1..21: mol_type = mRNA
tgataaagattaaccagaaga

SEQ ID NO: 231 - RNA, 21 nt, Homo sapiens
  source 1..21: mol_type = mRNA
gattaaccagaagaaaacaag

SEQ ID NO: 232 - RNA, 21 nt, Homo sapiens
  source 1..21: mol_type = mRNA
attaaccagaagaaaacaagg

SEQ ID NO: 233 - RNA, 21 nt, Homo sapiens
  source 1..21: mol_type = mRNA
ttaaccagaagaaaacaagga

SEQ ID NO: 234 - RNA, 21 nt, Homo sapiens
  source 1..21: mol_type = mRNA
acaaggagggaaacaaccgca

SEQ ID NO: 235 - RNA, 21 nt, Homo sapiens
  source 1..21: mol_type = mRNA
caaggagggaaacaaccgcag

SEQ ID NO: 236 - RNA, 21 nt, Homo sapiens
  source 1..21: mol_type = mRNA
ggagggaaacaaccgcagcct

SEQ ID NO: 237 - RNA, 21 nt, Homo sapiens
  source 1..21: mol_type = mRNA
gggaaacaaccgcagcctgta

SEQ ID NO: 238 - RNA, 21 nt, Homo sapiens
  source 1..21: mol_type = mRNA
caggagtcgcgcgctaggggc

SEQ ID NO: 239 - RNA, 21 nt, Homo sapiens
  source 1..21: mol_type = mRNA
ggggccggggccggggccggg

SEQ ID NO: 240 - RNA, 21 nt, Homo sapiens
  source 1..21: mol_type = mRNA
gggccggggccggggccgggg

SEQ ID NO: 241 - RNA, 21 nt, Homo sapiens
  source 1..21: mol_type = mRNA
ggccggggccggggccggggc

SEQ ID NO: 242 - RNA, 21 nt, Homo sapiens
  source 1..21: mol_type = mRNA
gccggggccggggccggggcc

SEQ ID NO: 243 - RNA, 21 nt, Homo sapiens
  source 1..21: mol_type = mRNA
ccggggccggggccggggccg

SEQ ID NO: 244 - RNA, 21 nt, Homo sapiens
  source 1..21: mol_type = mRNA
cggggccggggccggggccgg

SEQ ID NO: 245 - RNA, 21 nt, Homo sapiens
  source 1..21: mol_type = mRNA
ctcagagctcgacgcattttt

SEQ ID NO: 246 - RNA, 21 nt, Homo sapiens
  source 1..21: mol_type = mRNA
tcagagctcgacgcattttta

SEQ ID NO: 247 - RNA, 21 nt, Homo sapiens
  source 1..21: mol_type = mRNA
tcgacgcatttttactttccc

SEQ ID NO: 248 - RNA, 21 nt, Homo sapiens
  source 1..21: mol_type = mRNA
gacgcatttttactttccctc

SEQ ID NO: 249 - RNA, 21 nt, Homo sapiens
  source 1..21: mol_type = mRNA
ttccctctcatttctctgacc

SEQ ID NO: 250 - RNA, 21 nt, Homo sapiens
  source 1..21: mol_type = mRNA
cctctcatttctctgaccgaa

SEQ ID NO: 251 - RNA, 21 nt, Homo sapiens
  source 1..21: mol_type = mRNA
ctctcatttctctgaccgaag

SEQ ID NO: 252 - RNA, 21 nt, Homo sapiens
  source 1..21: mol_type = mRNA
ttctctgaccgaagctgggtg

SEQ ID NO: 253 - RNA, 21 nt, Homo sapiens
  source 1..21: mol_type = mRNA
ctttcgcctctagcgactggt

SEQ ID NO: 254 - RNA, 21 nt, Homo sapiens
  source 1..21: mol_type = mRNA
tttcgcctctagcgactggtg

SEQ ID NO: 255 - RNA, 21 nt, Homo sapiens
  source 1..21: mol_type = mRNA
tcgcctctagcgactggtgga

SEQ ID NO: 256 - RNA, 21 nt, Homo sapiens
  source 1..21: mol_type = mRNA
cgcctctagcgactggtggaa

SEQ ID NO: 257 - RNA, 21 nt, Homo sapiens
  source 1..21: mol_type = mRNA
ttcccgccctcagtacccgag

SEQ ID NO: 258 - RNA, 21 nt, Homo sapiens
  source 1..21: mol_type = mRNA
ggagacgcctgcacaatttca

SEQ ID NO: 259 - RNA, 21 nt, Homo sapiens
  source 1..21: mol_type = mRNA
cgcctgcacaatttcagccca

SEQ ID NO: 260 - RNA, 21 nt, Homo sapiens
  source 1..21: mol_type = mRNA
tctagagagtggtgatgactt

SEQ ID NO: 261 - RNA, 21 nt, Homo sapiens
  source 1..21: mol_type = mRNA
tagagagtggtgatgacttgc

SEQ ID NO: 262 - RNA, 21 nt, Homo sapiens
  source 1..21: mol_type = mRNA
ggtgatgacttgcatatgagg

SEQ ID NO: 263 - RNA, 21 nt, Homo sapiens
  source 1..21: mol_type = mRNA
tgatgacttgcatatgagggc

SEQ ID NO: 264 - RNA, 21 nt, Homo sapiens
  source 1..21: mol_type = mRNA
gatgacttgcatatgagggca

SEQ ID NO: 265 - RNA, 21 nt, Homo sapiens
  source 1..21: mol_type = mRNA
cttgcatatgagggcagcaat

SEQ ID NO: 266 - RNA, 21 nt, Homo sapiens
  source 1..21: mol_type = mRNA
atatgagggcagcaatgcaag

SEQ ID NO: 267 - RNA, 21 nt, Homo sapiens
  source 1..21: mol_type = mRNA
tgagggcagcaatgcaagtcg

SEQ ID NO: 268 - RNA, 21 nt, Homo sapiens
  source 1..21: mol_type = mRNA
ggcagcaatgcaagtcggtgt

SEQ ID NO: 269 - RNA, 21 nt, Homo sapiens
  source 1..21: mol_type = mRNA
gtgggacatgacctggttgct

SEQ ID NO: 270 - RNA, 21 nt, Homo sapiens
  source 1..21: mol_type = mRNA
cctggttgcttcacagctccg

SEQ ID NO: 271 - RNA, 21 nt, Homo sapiens
  source 1..21: mol_type = mRNA
tggttgcttcacagctccgag

SEQ ID NO: 272 - RNA, 21 nt, Homo sapiens
  source 1..21: mol_type = mRNA
ttgcttcacagctccgagatg

SEQ ID NO: 273 - RNA, 21 nt, Homo sapiens
  source 1..21: mol_type = mRNA
tcacagctccgagatgacaca

SEQ ID NO: 274 - RNA, 21 nt, Homo sapiens
  source 1..21: mol_type = mRNA
ctccgagatgacacagacttg

SEQ ID NO: 275 - RNA, 21 nt, Homo sapiens
  source 1..21: mol_type = mRNA
tccgagatgacacagacttgc

SEQ ID NO: 276 - RNA, 21 nt, Homo sapiens
  source 1..21: mol_type = mRNA
ccgagatgacacagacttgct

SEQ ID NO: 277 - RNA, 21 nt, Homo sapiens
  source 1..21: mol_type = mRNA
agatgacacagacttgcttaa

SEQ ID NO: 278 - RNA, 21 nt, Homo sapiens
  source 1..21: mol_type = mRNA
atgacacagacttgcttaaag

SEQ ID NO: 279 - RNA, 21 nt, Homo sapiens
  source 1..21: mol_type = mRNA
cacagacttgcttaaaggaag

SEQ ID NO: 280 - RNA, 21 nt, Homo sapiens
  source 1..21: mol_type = mRNA
cttgcttaaaggaagtgacta

SEQ ID NO: 281 - RNA, 21 nt, Homo sapiens
  source 1..21: mol_type = mRNA
tgcttaaaggaagtgactatt

SEQ ID NO: 282 - RNA, 21 nt, Homo sapiens
  source 1..21: mol_type = mRNA
ttaaaggaagtgactattgtg

SEQ ID NO: 283 - RNA, 21 nt, Homo sapiens
  source 1..21: mol_type = mRNA
taaaggaagtgactattgtga

SEQ ID NO: 284 - RNA, 21 nt, Homo sapiens
  source 1..21: mol_type = mRNA
gaagtgactattgtgacttgg

SEQ ID NO: 285 - RNA, 21 nt, Homo sapiens
  source 1..21: mol_type = mRNA
gtgactattgtgacttgggca

SEQ ID NO: 286 - RNA, 21 nt, Homo sapiens
  source 1..21: mol_type = mRNA
gactattgtgacttgggcatc

SEQ ID NO: 287 - RNA, 21 nt, Homo sapiens
  source 1..21: mol_type = mRNA
ttgtgacttgggcatcacttg

SEQ ID NO: 288 - RNA, 21 nt, Homo sapiens
  source 1..21: mol_type = mRNA
tgtgacttgggcatcacttga

SEQ ID NO: 289 - RNA, 21 nt, Homo sapiens
  source 1..21: mol_type = mRNA
gtgacttgggcatcacttgac

SEQ ID NO: 290 - RNA, 21 nt, Homo sapiens
  source 1..21: mol_type = mRNA
tgacttgggcatcacttgact

SEQ ID NO: 291 - RNA, 21 nt, Homo sapiens
  source 1..21: mol_type = mRNA
cttgggcatcacttgactgat

SEQ ID NO: 292 - RNA, 21 nt, Homo sapiens
  source 1..21: mol_type = mRNA
ttgggcatcacttgactgatg

SEQ ID NO: 293 - RNA, 21 nt, Homo sapiens
  source 1..21: mol_type = mRNA
tcacttgactgatggtaatca

SEQ ID NO: 294 - RNA, 21 nt, Homo sapiens
  source 1..21: mol_type = mRNA
acttgactgatggtaatcagt

SEQ ID NO: 295 - RNA, 21 nt, Homo sapiens
  source 1..21: mol_type = mRNA
ttgactgatggtaatcagttg

SEQ ID NO: 296 - RNA, 21 nt, Homo sapiens
  source 1..21: mol_type = mRNA
tgactgatggtaatcagttgt

SEQ ID NO: 297 - RNA, 21 nt, Homo sapiens
  source 1..21: mol_type = mRNA
gactgatggtaatcagttgtc

SEQ ID NO: 298 - RNA, 21 nt, Homo sapiens
  source 1..21: mol_type = mRNA
tgatggtaatcagttgtctaa

SEQ ID NO: 299 - RNA, 21 nt, Homo sapiens
  source 1..21: mol_type = mRNA
gatggtaatcagttgtctaaa

SEQ ID NO: 300 - RNA, 21 nt, Homo sapiens
  source 1..21: mol_type = mRNA
ggtaatcagttgtctaaagaa

SEQ ID NO: 301 - RNA, 21 nt, Homo sapiens
  source 1..21: mol_type = mRNA
atcagttgtctaaagaagtgc

SEQ ID NO: 302 - RNA, 21 nt, Homo sapiens
  source 1..21: mol_type = mRNA
tcagttgtctaaagaagtgca

SEQ ID NO: 303 - RNA, 21 nt, Homo sapiens
  source 1..21: mol_type = mRNA
gttgtctaaagaagtgcacag

SEQ ID NO: 304 - RNA, 21 nt, Homo sapiens
  source 1..21: mol_type = mRNA
ttgtctaaagaagtgcacaga

SEQ ID NO: 305 - RNA, 21 nt, Homo sapiens
  source 1..21: mol_type = mRNA
tgtctaaagaagtgcacagat

SEQ ID NO: 306 - RNA, 21 nt, Homo sapiens
  source 1..21: mol_type = mRNA
gtctaaagaagtgcacagatt

SEQ ID NO: 307 - RNA, 21 nt, Homo sapiens
  source 1..21: mol_type = mRNA
tctaaagaagtgcacagatta

SEQ ID NO: 308 - RNA, 21 nt, Homo sapiens
  source 1..21: mol_type = mRNA
ctaaagaagtgcacagattac

SEQ ID NO: 309 - RNA, 21 nt, Homo sapiens
  source 1..21: mol_type = mRNA
taaagaagtgcacagattaca

SEQ ID NO: 310 - RNA, 21 nt, Homo sapiens
  source 1..21: mol_type = mRNA
agtgcacagattacatgtccg

SEQ ID NO: 311 - RNA, 21 nt, Homo sapiens
  source 1..21: mol_type = mRNA
tgcacagattacatgtccgtg

SEQ ID NO: 312 - RNA, 21 nt, Homo sapiens
  source 1..21: mol_type = mRNA
agattacatgtccgtgtgctc

SEQ ID NO: 313 - RNA, 21 nt, Homo sapiens
  source 1..21: mol_type = mRNA
cgtgtgctcattgggtctatc

SEQ ID NO: 314 - RNA, 21 nt, Homo sapiens
  source 1..21: mol_type = mRNA
tgtgctcattgggtctatctg

SEQ ID NO: 315 - RNA, 21 nt, Homo sapiens
  source 1..21: mol_type = mRNA
tcattgggtctatctggccgc

SEQ ID NO: 316 - RNA, 21 nt, Homo sapiens
  source 1..21: mol_type = mRNA
gtctatctggccgcgttgaac

SEQ ID NO: 317 - RNA, 21 nt, Homo sapiens
  source 1..21: mol_type = mRNA
ccgcgttgaacaccaccaggc

SEQ ID NO: 318 - RNA, 21 nt, Homo sapiens
  source 1..21: mol_type = mRNA
gttgaacaccaccaggctttg

SEQ ID NO: 319 - RNA, 21 nt, Homo sapiens
  source 1..21: mol_type = mRNA
tgaacaccaccaggctttgta

SEQ ID NO: 320 - RNA, 21 nt, Homo sapiens
  source 1..21: mol_type = mRNA
gaacaccaccaggctttgtat

SEQ ID NO: 321 - RNA, 21 nt, Homo sapiens
  source 1..21: mol_type = mRNA
tttgtattcagaaacaggagg

SEQ ID NO: 322 - RNA, 21 nt, Homo sapiens
  source 1..21: mol_type = mRNA
cagaaacaggagggaggtcct

SEQ ID NO: 323 - RNA, 21 nt, Homo sapiens
  source 1..21: mol_type = mRNA
gagggaggtcctgcactttcc

SEQ ID NO: 324 - RNA, 21 nt, Homo sapiens
  source 1..21: mol_type = mRNA
ggtggccctttcagatgcaat

SEQ ID NO: 325 - RNA, 21 nt, Homo sapiens
  source 1..21: mol_type = mRNA
gtggccctttcagatgcaatc

SEQ ID NO: 326 - RNA, 21 nt, Homo sapiens
  source 1..21: mol_type = mRNA
tggccctttcagatgcaatcg

SEQ ID NO: 327 - RNA, 21 nt, Homo sapiens
  source 1..21: mol_type = mRNA
ccctttcagatgcaatcgaga

SEQ ID NO: 328 - RNA, 21 nt, Homo sapiens
  source 1..21: mol_type = mRNA
tttcagatgcaatcgagattg

SEQ ID NO: 329 - RNA, 21 nt, Homo sapiens
  source 1..21: mol_type = mRNA
cagatgcaatcgagattgtta

SEQ ID NO: 330 - RNA, 21 nt, Homo sapiens
  source 1..21: mol_type = mRNA
aatcgagattgttaggctctg

SEQ ID NO: 331 - RNA, 21 nt, Homo sapiens
  source 1..21: mol_type = mRNA
atcgagattgttaggctctgg

SEQ ID NO: 332 - RNA, 21 nt, Homo sapiens
  source 1..21: mol_type = mRNA
tcgagattgttaggctctggg

SEQ ID NO: 333 - RNA, 21 nt, Homo sapiens
  source 1..21: mol_type = mRNA
cgagattgttaggctctggga

SEQ ID NO: 334 - RNA, 21 nt, Homo sapiens
  source 1..21: mol_type = mRNA
gagattgttaggctctgggag

SEQ ID NO: 335 - RNA, 21 nt, Homo sapiens
  source 1..21: mol_type = mRNA
ttgttaggctctgggagagta

SEQ ID NO: 336 - RNA, 21 nt, Homo sapiens
  source 1..21: mol_type = mRNA
gttaggctctgggagagtagt

SEQ ID NO: 337 - RNA, 21 nt, Homo sapiens
  source 1..21: mol_type = mRNA
ttaggctctgggagagtagtt

SEQ ID NO: 338 - RNA, 21 nt, Homo sapiens
  source 1..21: mol_type = mRNA
taggctctgggagagtagttg

SEQ ID NO: 339 - RNA, 21 nt, Homo sapiens
  source 1..21: mol_type = mRNA
ggctctgggagagtagttgcc

SEQ ID NO: 340 - RNA, 21 nt, Homo sapiens
  source 1..21: mol_type = mRNA
ctgggagagtagttgcctggt

SEQ ID NO: 341 - RNA, 21 nt, Homo sapiens
  source 1..21: mol_type = mRNA
gggagagtagttgcctggttg

SEQ ID NO: 342 - RNA, 21 nt, Homo sapiens
  source 1..21: mol_type = mRNA
gagagtagttgcctggttgtg

SEQ ID NO: 343 - RNA, 21 nt, Homo sapiens
  source 1..21: mol_type = mRNA
ttgcctggttgtggcagttgg

SEQ ID NO: 344 - RNA, 21 nt, Homo sapiens
  source 1..21: mol_type = mRNA
tgcctggttgtggcagttggt

SEQ ID NO: 345 - RNA, 21 nt, Homo sapiens
  source 1..21: mol_type = mRNA
gcctggttgtggcagttggta

SEQ ID NO: 346 - RNA, 21 nt, Homo sapiens
  source 1..21: mol_type = mRNA
cctggttgtggcagttggtaa

SEQ ID NO: 347 - RNA, 21 nt, Homo sapiens
  source 1..21: mol_type = mRNA
ctggttgtggcagttggtaaa

SEQ ID NO: 348 - RNA, 21 nt, Homo sapiens
  source 1..21: mol_type = mRNA
tggttgtggcagttggtaaat

SEQ ID NO: 349 - RNA, 21 nt, Homo sapiens
  source 1..21: mol_type = mRNA
ttgtggcagttggtaaatttc

SEQ ID NO: 350 - RNA, 21 nt, Homo sapiens
  source 1..21: mol_type = mRNA
tgtggcagttggtaaatttct

SEQ ID NO: 351 - RNA, 21 nt, Homo sapiens
  source 1..21: mol_type = mRNA
gtggcagttggtaaatttcta

SEQ ID NO: 352 - RNA, 21 nt, Homo sapiens
  source 1..21: mol_type = mRNA
tggcagttggtaaatttctat

SEQ ID NO: 353 - RNA, 21 nt, Homo sapiens
  source 1..21: mol_type = mRNA
ggcagttggtaaatttctatt

SEQ ID NO: 354 - RNA, 21 nt, Homo sapiens
  source 1..21: mol_type = mRNA
gcagttggtaaatttctattc

SEQ ID NO: 355 - RNA, 21 nt, Homo sapiens
  source 1..21: mol_type = mRNA
cagttggtaaatttctattca

SEQ ID NO: 356 - RNA, 21 nt, Homo sapiens
  source 1..21: mol_type = mRNA
tctattcaaacagttgccatg

SEQ ID NO: 357 - RNA, 21 nt, Homo sapiens
  source 1..21: mol_type = mRNA
tattcaaacagttgccatgca

SEQ ID NO: 358 - RNA, 21 nt, Homo sapiens
  source 1..21: mol_type = mRNA
attcaaacagttgccatgcac

SEQ ID NO: 359 - RNA, 21 nt, Homo sapiens
  source 1..21: mol_type = mRNA
ttcaaacagttgccatgcacc

SEQ ID NO: 360 - RNA, 21 nt, Homo sapiens
  source 1..21: mol_type = mRNA
cagttgccatgcaccagttgt

SEQ ID NO: 361 - RNA, 21 nt, Homo sapiens
  source 1..21: mol_type = mRNA
agttgccatgcaccagttgtt

SEQ ID NO: 362 - RNA, 21 nt, Homo sapiens
  source 1..21: mol_type = mRNA
ttgccatgcaccagttgttca

SEQ ID NO: 363 - RNA, 21 nt, Homo sapiens
  source 1..21: mol_type = mRNA
tgccatgcaccagttgttcac

SEQ ID NO: 364 - RNA, 21 nt, Homo sapiens
  source 1..21: mol_type = mRNA
gccatgcaccagttgttcaca

SEQ ID NO: 365 - RNA, 21 nt, Homo sapiens
  source 1..21: mol_type = mRNA
atgcaccagttgttcacaaca

SEQ ID NO: 366 - RNA, 21 nt, Homo sapiens
  source 1..21: mol_type = mRNA
gttgttcacaacaagggtacg

SEQ ID NO: 367 - RNA, 21 nt, Homo sapiens
  source 1..21: mol_type = mRNA
ttcacaacaagggtacgtaat

SEQ ID NO: 368 - RNA, 21 nt, Homo sapiens
  source 1..21: mol_type = mRNA
tcacaacaagggtacgtaatc

SEQ ID NO: 369 - RNA, 21 nt, Homo sapiens
  source 1..21: mol_type = mRNA
cacaacaagggtacgtaatct

SEQ ID NO: 370 - RNA, 21 nt, Homo sapiens
  source 1..21: mol_type = mRNA
caagggtacgtaatctgtctg

SEQ ID NO: 371 - RNA, 21 nt, Homo sapiens
  source 1..21: mol_type = mRNA
agggtacgtaatctgtctggc

SEQ ID NO: 372 - RNA, 21 nt, Homo sapiens
  source 1..21: mol_type = mRNA
gtacgtaatctgtctggcatt

SEQ ID NO: 373 - RNA, 21 nt, Homo sapiens
  source 1..21: mol_type = mRNA
tacgtaatctgtctggcatta

SEQ ID NO: 374 - RNA, 21 nt, Homo sapiens
  source 1..21: mol_type = mRNA
cgtaatctgtctggcattact

SEQ ID NO: 375 - RNA, 21 nt, Homo sapiens
  source 1..21: mol_type = mRNA
atctgtctggcattacttcta

SEQ ID NO: 376 - RNA, 21 nt, Homo sapiens
  source 1..21: mol_type = mRNA
ctgtctggcattacttctact

SEQ ID NO: 377 - RNA, 21 nt, Homo sapiens
  source 1..21: mol_type = mRNA
tctggcattacttctactttt

SEQ ID NO: 378 - RNA, 21 nt, Homo sapiens
  source 1..21: mol_type = mRNA
ctggcattacttctacttttg

SEQ ID NO: 379 - RNA, 21 nt, Homo sapiens
  source 1..21: mol_type = mRNA
tggcattacttctacttttgt

SEQ ID NO: 380 - RNA, 21 nt, Homo sapiens
  source 1..21: mol_type = mRNA
tctacttttgtacaaaggatc

SEQ ID NO: 381 - RNA, 21 nt, Homo sapiens
  source 1..21: mol_type = mRNA
ctacttttgtacaaaggatca

SEQ ID NO: 382 - RNA, 21 nt, Homo sapiens
  source 1..21: mol_type = mRNA
tacttttgtacaaaggatcaa

SEQ ID NO: 383 - RNA, 21 nt, Homo sapiens
  source 1..21: mol_type = mRNA
acttttgtacaaaggatcaaa

SEQ ID NO: 384 - RNA, 21 nt, Homo sapiens
  source 1..21: mol_type = mRNA
cttttgtacaaaggatcaaaa

SEQ ID NO: 385 - RNA, 16 nt, Synthetic construct
  source 1..16: mol_type = other RNA
ctgttgccaagacaga

SEQ ID NO: 386 - RNA, 16 nt, Synthetic construct
  source 1..16: mol_type = other RNA
gttgccaagacagaga

SEQ ID NO: 387 - RNA, 16 nt, Synthetic construct
  source 1..16: mol_type = other RNA
tgccaagacagagata

SEQ ID NO: 388 - RNA, 16 nt, Synthetic construct
  source 1..16: mol_type = other RNA
gccaagacagagatta

SEQ ID NO: 389 - RNA, 16 nt, Synthetic construct
  source 1..16: mol_type = other RNA
ccaagacagagattga

SEQ ID NO: 390 - RNA, 16 nt, Synthetic construct
  source 1..16: mol_type = other RNA
caagacagagattgca

SEQ ID NO: 391 - RNA, 16 nt, Synthetic construct
  source 1..16: mol_type = other RNA
aagacagagattgcta

SEQ ID NO: 392 - RNA, 16 nt, Synthetic construct
  source 1..16: mol_type = other RNA
acagagattgctttaa

SEQ ID NO: 393 - RNA, 16 nt, Synthetic construct
  source 1..16: mol_type = other RNA
gattgctttaagtgga

SEQ ID NO: 394 - RNA, 16 nt, Synthetic construct
  source 1..16: mol_type = other RNA
cctttattagcagcta

SEQ ID NO: 395 - RNA, 16 nt, Synthetic construct
  source 1..16: mol_type = other RNA
atattcttggtcctaa

SEQ ID NO: 396 - RNA, 16 nt, Synthetic construct
  source 1..16: mol_type = other RNA
tattcttggtcctaga

SEQ ID NO: 397 - RNA, 16 nt, Synthetic construct
  source 1..16: mol_type = other RNA
ttcttggtcctagaga

SEQ ID NO: 398 - RNA, 16 nt, Synthetic construct
  source 1..16: mol_type = other RNA
cttggtcctagagtaa

SEQ ID NO: 399 - RNA, 16 nt, Synthetic construct
  source 1..16: mol_type = other RNA
ggtcctagagtaagga

SEQ ID NO: 400 - RNA, 16 nt, Synthetic construct
  source 1..16: mol_type = other RNA
gtcctagagtaaggca

SEQ ID NO: 401 - RNA, 16 nt, Synthetic construct
  source 1..16: mol_type = other RNA
tcctagagtaaggcaa

SEQ ID NO: 402 - RNA, 16 nt, Synthetic construct
  source 1..16: mol_type = other RNA
tttgggctccaaagaa

SEQ ID NO: 403 - RNA, 16 nt, Synthetic construct
  source 1..16: mol_type = other RNA
ttgggctccaaagaca

SEQ ID NO: 404 - RNA, 16 nt, Synthetic construct
  source 1..16: mol_type = other RNA
tgggctccaaagacaa

SEQ ID NO: 405 - RNA, 16 nt, Synthetic construct
  source 1..16: mol_type = other RNA
gggctccaaagacaga

SEQ ID NO: 406 - RNA, 16 nt, Synthetic construct
  source 1..16: mol_type = other RNA
taacttttcttgccaa

SEQ ID NO: 407 - RNA, 16 nt, Synthetic construct
  source 1..16: mol_type = other RNA
cttttcttgccaacca

SEQ ID NO: 408 - RNA, 16 nt, Synthetic construct
  source 1..16: mol_type = other RNA
ttttcttgccaaccaa

SEQ ID NO: 409 - RNA, 16 nt, Synthetic construct
  source 1..16: mol_type = other RNA
tttcttgccaaccaca

SEQ ID NO: 410 - RNA, 16 nt, Synthetic construct
  source 1..16: mol_type = other RNA
ttgccaaccacactca

SEQ ID NO: 411 - RNA, 16 nt, Synthetic construct
  source 1..16: mol_type = other RNA
tgccaaccacactcta

SEQ ID NO: 412 - RNA, 16 nt, Synthetic construct
  source 1..16: mol_type = other RNA
gccaaccacactctaa

SEQ ID NO: 413 - RNA, 16 nt, Synthetic construct
  source 1..16: mol_type = other RNA
ccaaccacactctaaa

SEQ ID NO: 414 - RNA, 16 nt, Synthetic construct
  source 1..16: mol_type = other RNA
caaccacactctaaaa

SEQ ID NO: 415 - RNA, 16 nt, Synthetic construct
  source 1..16: mol_type = other RNA
aaccacactctaaata

SEQ ID NO: 416 - RNA, 16 nt, Synthetic construct
  source 1..16: mol_type = other RNA
accacactctaaatga

SEQ ID NO: 417 - RNA, 16 nt, Synthetic construct
  source 1..16: mol_type = other RNA
acactctaaatggaga

SEQ ID NO: 418 - RNA, 16 nt, Synthetic construct
  source 1..16: mol_type = other RNA
ttcgaaatgcagagaa

SEQ ID NO: 419 - RNA, 16 nt, Synthetic construct
  source 1..16: mol_type = other RNA
gaaatgcagagagtga

SEQ ID NO: 420 - RNA, 16 nt, Synthetic construct
  source 1..16: mol_type = other RNA
ggtgctatagatgtaa

SEQ ID NO: 421 - RNA, 16 nt, Synthetic construct
  source 1..16: mol_type = other RNA
gtgctatagatgtaaa

SEQ ID NO: 422 - RNA, 16 nt, Synthetic construct
  source 1..16: mol_type = other RNA
ctatagatgtaaagta

SEQ ID NO: 423 - RNA, 16 nt, Synthetic construct
  source 1..16: mol_type = other RNA
atagatgtaaagttta

SEQ ID NO: 424 - RNA, 16 nt, Synthetic construct
  source 1..16: mol_type = other RNA
cttgtctgaaaaggga

SEQ ID NO: 425 - RNA, 16 nt, Synthetic construct
  source 1..16: mol_type = other RNA
tctgaaaagggagtga

SEQ ID NO: 426 - RNA, 16 nt, Synthetic construct
  source 1..16: mol_type = other RNA
aaaagggagtgattaa

SEQ ID NO: 427 - RNA, 16 nt, Synthetic construct
  source 1..16: mol_type = other RNA
aaagggagtgattata

SEQ ID NO: 428 - RNA, 16 nt, Synthetic construct
  source 1..16: mol_type = other RNA
ggagtgattattgtta

SEQ ID NO: 429 - RNA, 16 nt, Synthetic construct
  source 1..16: mol_type = other RNA
tgatggaaactggaaa

SEQ ID NO: 430 - RNA, 16 nt, Synthetic construct
  source 1..16: mol_type = other RNA
acttccacagacagaa

SEQ ID NO: 431 - RNA, 16 nt, Synthetic construct
  source 1..16: mol_type = other RNA
cttccacagacagaaa

SEQ ID NO: 432 - RNA, 16 nt, Synthetic construct
  source 1..16: mol_type = other RNA
ccacagacagaactta

SEQ ID NO: 433 - RNA, 16 nt, Synthetic construct
  source 1..16: mol_type = other RNA
acagaacttagtttca

SEQ ID NO: 434 - RNA, 16 nt, Synthetic construct
  source 1..16: mol_type = other RNA
tctacctcccacttca

SEQ ID NO: 435 - RNA, 16 nt, Synthetic construct
  source 1..16: mol_type = other RNA
ctacctcccacttcaa

SEQ ID NO: 436 - RNA, 16 nt, Synthetic construct
  source 1..16: mol_type = other RNA
ataatccggaaaggaa

SEQ ID NO: 437 - RNA, 16 nt, Synthetic construct
  source 1..16: mol_type = other RNA
tccggaaaggaagaaa

SEQ ID NO: 438 - RNA, 16 nt, Synthetic construct
  source 1..16: mol_type = other RNA
ggaaaggaagaatata

SEQ ID NO: 439 - RNA, 16 nt, Synthetic construct
  source 1..16: mol_type = other RNA
aaaggaagaatatgga

SEQ ID NO: 440 - RNA, 16 nt, Synthetic construct
  source 1..16: mol_type = other RNA
agaatatggatgcata

SEQ ID NO: 441 - RNA, 16 nt, Synthetic construct
  source 1..16: mol_type = other RNA
atatggatgcataaga

SEQ ID NO: 442 - RNA, 16 nt, Synthetic construct
  source 1..16: mol_type = other RNA
atggatgcataaggaa

SEQ ID NO: 443 - RNA, 16 nt, Synthetic construct
  source 1..16: mol_type = other RNA
tggatgcataaggaaa

SEQ ID NO: 444 - RNA, 16 nt, Synthetic construct
  source 1..16: mol_type = other RNA
ggatgcataaggaaaa

SEQ ID NO: 445 - RNA, 16 nt, Synthetic construct
  source 1..16: mol_type = other RNA
tgcataaggaaagaca

SEQ ID NO: 446 - RNA, 16 nt, Synthetic construct
  source 1..16: mol_type = other RNA
gcataaggaaagacaa

SEQ ID NO: 447 - RNA, 16 nt, Synthetic construct
  source 1..16: mol_type = other RNA
cataaggaaagacaaa

SEQ ID NO: 448 - RNA, 16 nt, Synthetic construct
  source 1..16: mol_type = other RNA
agacaagaaaatgtca

SEQ ID NO: 449 - RNA, 16 nt, Synthetic construct
  source 1..16: mol_type = other RNA
aagaaaatgtccagaa

SEQ ID NO: 450 - RNA, 16 nt, Synthetic construct
  source 1..16: mol_type = other RNA
tagaaggcacagagaa

SEQ ID NO: 451 - RNA, 16 nt, Synthetic construct
  source 1..16: mol_type = other RNA
gaatggaagatcagga

SEQ ID NO: 452 - RNA, 16 nt, Synthetic construct
  source 1..16: mol_type = other RNA
tggaagatcagggtca

SEQ ID NO: 453 - RNA, 16 nt, Synthetic construct
  source 1..16: mol_type = other RNA
ggaagatcagggtcaa

SEQ ID NO: 454 - RNA, 16 nt, Synthetic construct
  source 1..16: mol_type = other RNA
gaagatcagggtcaga

SEQ ID NO: 455 - RNA, 16 nt, Synthetic construct
  source 1..16: mol_type = other RNA
aagatcagggtcagaa

SEQ ID NO: 456 - RNA, 16 nt, Synthetic construct
  source 1..16: mol_type = other RNA
agatcagggtcagaga

SEQ ID NO: 457 - RNA, 16 nt, Synthetic construct
  source 1..16: mol_type = other RNA
gatcagggtcagagta

SEQ ID NO: 458 - RNA, 16 nt, Synthetic construct
  source 1..16: mol_type = other RNA
atcagggtcagagtaa

SEQ ID NO: 459 - RNA, 16 nt, Synthetic construct
  source 1..16: mol_type = other RNA
tcagggtcagagtata

SEQ ID NO: 460 - RNA, 16 nt, Synthetic construct
  source 1..16: mol_type = other RNA
gggtcagagtattata

SEQ ID NO: 461 - RNA, 16 nt, Synthetic construct
  source 1..16: mol_type = other RNA
tcagagtattattcca

SEQ ID NO: 462 - RNA, 16 nt, Synthetic construct
  source 1..16: mol_type = other RNA
aagtgattcctgtaaa

SEQ ID NO: 463 - RNA, 16 nt, Synthetic construct
  source 1..16: mol_type = other RNA
atggaactgctttcaa

SEQ ID NO: 464 - RNA, 16 nt, Synthetic construct
  source 1..16: mol_type = other RNA
actgctttcatctata

SEQ ID NO: 465 - RNA, 16 nt, Synthetic construct
  source 1..16: mol_type = other RNA
ctatgaaatcacacaa

SEQ ID NO: 466 - RNA, 16 nt, Synthetic construct
  source 1..16: mol_type = other RNA
aaatcacacagtgtta

SEQ ID NO: 467 - RNA, 16 nt, Synthetic construct
  source 1..16: mol_type = other RNA
aagaaatagatataga

SEQ ID NO: 468 - RNA, 16 nt, Synthetic construct
  source 1..16: mol_type = other RNA
atatagctgatacaga

SEQ ID NO: 469 - RNA, 16 nt, Synthetic construct
  source 1..16: mol_type = other RNA
acagtactcaatgata

SEQ ID NO: 470 - RNA, 16 nt, Synthetic construct
  source 1..16: mol_type = other RNA
gctttcttctcaatga

SEQ ID NO: 471 - RNA, 16 nt, Synthetic construct
  source 1..16: mol_type = other RNA
ctttcttctcaatgca

SEQ ID NO: 472 - RNA, 16 nt, Synthetic construct
  source 1..16: mol_type = other RNA
tttcttctcaatgcca

SEQ ID NO: 473 - RNA, 16 nt, Synthetic construct
  source 1..16: mol_type = other RNA
caatgccatcagctca

SEQ ID NO: 474 - RNA, 16 nt, Synthetic construct
  source 1..16: mol_type = other RNA
aatgccatcagctcaa

SEQ ID NO: 475 - RNA, 16 nt, Synthetic construct
  source 1..16: mol_type = other RNA
atgccatcagctcaca

SEQ ID NO: 476 - RNA, 16 nt, Synthetic construct
  source 1..16: mol_type = other RNA
gccatcagctcacaca

SEQ ID NO: 477 - RNA, 16 nt, Synthetic construct
  source 1..16: mol_type = other RNA
ctgtggctgttccgta

SEQ ID NO: 478 - RNA, 16 nt, Synthetic construct
  source 1..16: mol_type = other RNA
tggctgttccgttgta

SEQ ID NO: 479 - RNA, 16 nt, Synthetic construct
  source 1..16: mol_type = other RNA
gctgttccgttgtaga

SEQ ID NO: 480 - RNA, 16 nt, Synthetic construct
  source 1..16: mol_type = other RNA
agtaggtagcagtgca

SEQ ID NO: 481 - RNA, 16 nt, Synthetic construct
  source 1..16: mol_type = other RNA
cagtgcagagaaagta

SEQ ID NO: 482 - RNA, 16 nt, Synthetic construct
  source 1..16: mol_type = other RNA
tgcagagaaagtaaaa

SEQ ID NO: 483 - RNA, 16 nt, Synthetic construct
  source 1..16: mol_type = other RNA
gcagagaaagtaaata

SEQ ID NO: 484 - RNA, 16 nt, Synthetic construct
  source 1..16: mol_type = other RNA
gaaagtaaataagata

SEQ ID NO: 485 - RNA, 16 nt, Synthetic construct
  source 1..16: mol_type = other RNA
tcagaacattatgcca

SEQ ID NO: 486 - RNA, 16 nt, Synthetic construct
  source 1..16: mol_type = other RNA
cagaatcatcatttaa

SEQ ID NO: 487 - RNA, 16 nt, Synthetic construct
  source 1..16: mol_type = other RNA
agaatcatcatttaaa

SEQ ID NO: 488 - RNA, 16 nt, Synthetic construct
  source 1..16: mol_type = other RNA
aaaggattcaactgga

SEQ ID NO: 489 - RNA, 16 nt, Synthetic construct
  source 1..16: mol_type = other RNA
gcaagtcatgtatgca

SEQ ID NO: 490 - RNA, 16 nt, Synthetic construct
  source 1..16: mol_type = other RNA
tatcccaccacacaca

SEQ ID NO: 491 - RNA, 16 nt, Synthetic construct
  source 1..16: mol_type = other RNA
ccctgtcatgaacata

SEQ ID NO: 492 - RNA, 16 nt, Synthetic construct
  source 1..16: mol_type = other RNA
cctgtcatgaacataa

SEQ ID NO: 493 - RNA, 16 nt, Synthetic construct
  source 1..16: mol_type = other RNA
tcatgaacatatttaa

SEQ ID NO: 494 - RNA, 16 nt, Synthetic construct
  source 1..16: mol_type = other RNA
atcagcgtagatacaa

SEQ ID NO: 495 - RNA, 16 nt, Synthetic construct
  source 1..16: mol_type = other RNA
gcgtagatacatgaga

SEQ ID NO: 496 - RNA, 16 nt, Synthetic construct
  source 1..16: mol_type = other RNA
tcaggatacgatcata

SEQ ID NO: 497 - RNA, 16 nt, Synthetic construct
  source 1..16: mol_type = other RNA
atctacactgacgaaa

SEQ ID NO: 498 - RNA, 16 nt, Synthetic construct
  source 1..16: mol_type = other RNA
aaagctttactcctga

SEQ ID NO: 499 - RNA, 16 nt, Synthetic construct
  source 1..16: mol_type = other RNA
ctcctgatttgaataa

SEQ ID NO: 500 - RNA, 16 nt, Synthetic construct
  source 1..16: mol_type = other RNA
agatgtcttacacaga

SEQ ID NO: 501 - RNA, 16 nt, Synthetic construct
  source 1..16: mol_type = other RNA
ttacacagagacacta

SEQ ID NO: 502 - RNA, 16 nt, Synthetic construct
  source 1..16: mol_type = other RNA
tacacagagacactca

SEQ ID NO: 503 - RNA, 16 nt, Synthetic construct
  source 1..16: mol_type = other RNA
acacagagacactcta

SEQ ID NO: 504 - RNA, 16 nt, Synthetic construct
  source 1..16: mol_type = other RNA
cacagagacactctaa

SEQ ID NO: 505 - RNA, 16 nt, Synthetic construct
  source 1..16: mol_type = other RNA
acagagacactctaga

SEQ ID NO: 506 - RNA, 16 nt, Synthetic construct
  source 1..16: mol_type = other RNA
cagagacactctagta

SEQ ID NO: 507 - RNA, 16 nt, Synthetic construct
  source 1..16: mol_type = other RNA
agagacactctagtga

SEQ ID NO: 508 - RNA, 16 nt, Synthetic construct
  source 1..16: mol_type = other RNA
gagacactctagtgaa

SEQ ID NO: 509 - RNA, 16 nt, Synthetic construct
  source 1..16: mol_type = other RNA
agacactctagtgaaa

SEQ ID NO: 510 - RNA, 16 nt, Synthetic construct
  source 1..16: mol_type = other RNA
gacactctagtgaaaa

SEQ ID NO: 511 - RNA, 16 nt, Synthetic construct
  source 1..16: mol_type = other RNA
gtactttccttgcaca

SEQ ID NO: 512 - RNA, 16 nt, Synthetic construct
  source 1..16: mol_type = other RNA
tttccttgcacagtta

SEQ ID NO: 513 - RNA, 16 nt, Synthetic construct
  source 1..16: mol_type = other RNA
acagaaaagccttgaa

SEQ ID NO: 514 - RNA, 16 nt, Synthetic construct
  source 1..16: mol_type = other RNA
aaaagccttgacacta

SEQ ID NO: 515 - RNA, 16 nt, Synthetic construct
  source 1..16: mol_type = other RNA
aaagccttgacactaa

SEQ ID NO: 516 - RNA, 16 nt, Synthetic construct
  source 1..16: mol_type = other RNA
aagccttgacactaaa

SEQ ID NO: 517 - RNA, 16 nt, Synthetic construct
  source 1..16: mol_type = other RNA
acgcagaagggaaaaa

SEQ ID NO: 518 - RNA, 16 nt, Synthetic construct
  source 1..16: mol_type = other RNA
cgcagaagggaaaaaa

SEQ ID NO: 519 - RNA, 16 nt, Synthetic construct
  source 1..16: mol_type = other RNA
tgatttaacagcagaa

SEQ ID NO: 520 - RNA, 16 nt, Synthetic construct
  source 1..16: mol_type = other RNA
gatttaacagcagaga

SEQ ID NO: 521 - RNA, 16 nt, Synthetic construct
  source 1..16: mol_type = other RNA
atttaacagcagagga

SEQ ID NO: 522 - RNA, 16 nt, Synthetic construct
  source 1..16: mol_type = other RNA
tttaacagcagaggga

SEQ ID NO: 523 - RNA, 16 nt, Synthetic construct
  source 1..16: mol_type = other RNA
ttaacagcagagggca

SEQ ID NO: 524 - RNA, 16 nt, Synthetic construct
  source 1..16: mol_type = other RNA
taacagcagagggcga

SEQ ID NO: 525 - RNA, 16 nt, Synthetic construct
  source 1..16: mol_type = other RNA
acagcagagggcgata

SEQ ID NO: 526 - RNA, 16 nt, Synthetic construct
  source 1..16: mol_type = other RNA
cagcagagggcgatca

SEQ ID NO: 527 - RNA, 16 nt, Synthetic construct
  source 1..16: mol_type = other RNA
gcagagggcgatctta

SEQ ID NO: 528 - RNA, 16 nt, Synthetic construct
  source 1..16: mol_type = other RNA
cagagggcgatcttaa

SEQ ID NO: 529 - RNA, 16 nt, Synthetic construct
  source 1..16: mol_type = other RNA
gagggcgatcttaaca

SEQ ID NO: 530 - RNA, 16 nt, Synthetic construct
  source 1..16: mol_type = other RNA
gcgatcttaacataaa

SEQ ID NO: 531 - RNA, 16 nt, Synthetic construct
  source 1..16: mol_type = other RNA
cgatcttaacataata

SEQ ID NO: 532 - RNA, 16 nt, Synthetic construct
  source 1..16: mol_type = other RNA
gatcttaacataataa

SEQ ID NO: 533 - RNA, 16 nt, Synthetic construct
  source 1..16: mol_type = other RNA
atcttaacataataaa

SEQ ID NO: 534 - RNA, 16 nt, Synthetic construct
  source 1..16: mol_type = other RNA
ttaacataataatgga

SEQ ID NO: 535 - RNA, 16 nt, Synthetic construct
  source 1..16: mol_type = other RNA
taacataataatggca

SEQ ID NO: 536 - RNA, 16 nt, Synthetic construct
  source 1..16: mol_type = other RNA
acataataatggctca

SEQ ID NO: 537 - RNA, 16 nt, Synthetic construct
  source 1..16: mol_type = other RNA
cataataatggctcta

SEQ ID NO: 538 - RNA, 16 nt, Synthetic construct
  source 1..16: mol_type = other RNA
tggctgagaaaattaa

SEQ ID NO: 539 - RNA, 16 nt, Synthetic construct
  source 1..16: mol_type = other RNA
gctgagaaaattaaaa

SEQ ID NO: 540 - RNA, 16 nt, Synthetic construct
  source 1..16: mol_type = other RNA
caggcctacactctta

SEQ ID NO: 541 - RNA, 16 nt, Synthetic construct
  source 1..16: mol_type = other RNA
gacctttctacactaa

SEQ ID NO: 542 - RNA, 16 nt, Synthetic construct
  source 1..16: mol_type = other RNA
acctttctacactaga

SEQ ID NO: 543 - RNA, 16 nt, Synthetic construct
  source 1..16: mol_type = other RNA
cctttctacactagta

SEQ ID NO: 544 - RNA, 16 nt, Synthetic construct
  source 1..16: mol_type = other RNA
ctttctacactagtga

SEQ ID NO: 545 - RNA, 16 nt, Synthetic construct
  source 1..16: mol_type = other RNA
gaacgagatgttctaa

SEQ ID NO: 546 - RNA, 16 nt, Synthetic construct
  source 1..16: mol_type = other RNA
acttaataagcctata

SEQ ID NO: 547 - RNA, 16 nt, Synthetic construct
  source 1..16: mol_type = other RNA
taataagcctattcca

SEQ ID NO: 548 - RNA, 16 nt, Synthetic construct
  source 1..16: mol_type = other RNA
agcctattccatcaca

SEQ ID NO: 549 - RNA, 16 nt, Synthetic construct
  source 1..16: mol_type = other RNA
attgcagttaagtaaa

SEQ ID NO: 550 - RNA, 16 nt, Synthetic construct
  source 1..16: mol_type = other RNA
ttaagtaagttacaca

SEQ ID NO: 551 - RNA, 16 nt, Synthetic construct
  source 1..16: mol_type = other RNA
ttacactacagttcta

SEQ ID NO: 552 - RNA, 16 nt, Synthetic construct
  source 1..16: mol_type = other RNA
gcatcattacattgga

SEQ ID NO: 553 - RNA, 16 nt, Synthetic construct
  source 1..16: mol_type = other RNA
ttgggatacagaccta

SEQ ID NO: 554 - RNA, 16 nt, Synthetic construct
  source 1..16: mol_type = other RNA
tgggatacagacctaa

SEQ ID NO: 555 - RNA, 16 nt, Synthetic construct
  source 1..16: mol_type = other RNA
ggatacagacctatga

SEQ ID NO: 556 - RNA, 16 nt, Synthetic construct
  source 1..16: mol_type = other RNA
atacagacctatgtta

SEQ ID NO: 557 - RNA, 16 nt, Synthetic construct
  source 1..16: mol_type = other RNA
tacagacctatgttta

SEQ ID NO: 558 - RNA, 16 nt, Synthetic construct
  source 1..16: mol_type = other RNA
acctatgtttacaata

SEQ ID NO: 559 - RNA, 16 nt, Synthetic construct
  source 1..16: mol_type = other RNA
ctatgtttacaatata

SEQ ID NO: 560 - RNA, 16 nt, Synthetic construct
  source 1..16: mol_type = other RNA
tgattcatggtttaca

SEQ ID NO: 561 - RNA, 16 nt, Synthetic construct
  source 1..16: mol_type = other RNA
ctttctatcttttgga

SEQ ID NO: 562 - RNA, 16 nt, Synthetic construct
  source 1..16: mol_type = other RNA
acagaaagaaagtgaa

SEQ ID NO: 563 - RNA, 16 nt, Synthetic construct
  source 1..16: mol_type = other RNA
aagcagatgtttaata

SEQ ID NO: 564 - RNA, 16 nt, Synthetic construct
  source 1..16: mol_type = other RNA
agatgtttaattggaa

SEQ ID NO: 565 - RNA, 16 nt, Synthetic construct
  source 1..16: mol_type = other RNA
ggattcagtctgtaga

SEQ ID NO: 566 - RNA, 16 nt, Synthetic construct
  source 1..16: mol_type = other RNA
tctgtagaaatgtcta

SEQ ID NO: 567 - RNA, 16 nt, Synthetic construct
  source 1..16: mol_type = other RNA
gtgaaccacagttaga

SEQ ID NO: 568 - RNA, 16 nt, Synthetic construct
  source 1..16: mol_type = other RNA
agttgagctctgtaaa

SEQ ID NO: 569 - RNA, 16 nt, Synthetic construct
  source 1..16: mol_type = other RNA
gttgagctctgtaaaa

SEQ ID NO: 570 - RNA, 16 nt, Synthetic construct
  source 1..16: mol_type = other RNA
tgagctctgtaaaaga

SEQ ID NO: 571 - RNA, 16 nt, Synthetic construct
  source 1..16: mol_type = other RNA
agctctgtaaaaggaa

SEQ ID NO: 572 - RNA, 16 nt, Synthetic construct
  source 1..16: mol_type = other RNA
gctctgtaaaaggaaa

SEQ ID NO: 573 - RNA, 16 nt, Synthetic construct
  source 1..16: mol_type = other RNA
ccaaattgaaatgtga

SEQ ID NO: 574 - RNA, 16 nt, Synthetic construct
  source 1..16: mol_type = other RNA
acctcctgtgccttta

SEQ ID NO: 575 - RNA, 16 nt, Synthetic construct
  source 1..16: mol_type = other RNA
atttcactggtcagta

SEQ ID NO: 576 - RNA, 16 nt, Synthetic construct
  source 1..16: mol_type = other RNA
ccatgtacctgcttta

SEQ ID NO: 577 - RNA, 16 nt, Synthetic construct
  source 1..16: mol_type = other RNA
gctaggaaagagagga

SEQ ID NO: 578 - RNA, 16 nt, Synthetic construct
  source 1..16: mol_type = other RNA
acagcgacaagttcca

SEQ ID NO: 579 - RNA, 16 nt, Synthetic construct
  source 1..16: mol_type = other RNA
cagcgacaagttccga

SEQ ID NO: 580 - RNA, 16 nt, Synthetic construct
  source 1..16: mol_type = other RNA
acaagttccgcccaca

SEQ ID NO: 581 - RNA, 16 nt, Synthetic construct
  source 1..16: mol_type = other RNA
agttccgcccacgtaa

SEQ ID NO: 582 - RNA, 16 nt, Synthetic construct
  source 1..16: mol_type = other RNA
gttccgcccacgtaaa

SEQ ID NO: 583 - RNA, 16 nt, Synthetic construct
  source 1..16: mol_type = other RNA
ccgcccacgtaaaaga

SEQ ID NO: 584 - RNA, 16 nt, Synthetic construct
  source 1..16: mol_type = other RNA
cccacgtaaaagatga

SEQ ID NO: 585 - RNA, 16 nt, Synthetic construct
  source 1..16: mol_type = other RNA
ccacgtaaaagatgaa

SEQ ID NO: 586 - RNA, 16 nt, Synthetic construct
  source 1..16: mol_type = other RNA
cacgtaaaagatgaca

SEQ ID NO: 587 - RNA, 16 nt, Synthetic construct
  source 1..16: mol_type = other RNA
acgtaaaagatgacga

SEQ ID NO: 588 - RNA, 16 nt, Synthetic construct
  source 1..16: mol_type = other RNA
taaaagatgacgctta

SEQ ID NO: 589 - RNA, 16 nt, Synthetic construct
  source 1..16: mol_type = other RNA
tcagccgtccctgcta

SEQ ID NO: 590 - RNA, 16 nt, Synthetic construct
  source 1..16: mol_type = other RNA
cccggttgcttctcta

SEQ ID NO: 591 - RNA, 16 nt, Synthetic construct
  source 1..16: mol_type = other RNA
gcaagagcaggtgtga

SEQ ID NO: 592 - RNA, 16 nt, Synthetic construct
  source 1..16: mol_type = other RNA
gcaggtgtgggtttaa

SEQ ID NO: 593 - RNA, 16 nt, Synthetic construct
  source 1..16: mol_type = other RNA
tgggtttaggaggtga

SEQ ID NO: 594 - RNA, 16 nt, Synthetic construct
  source 1..16: mol_type = other RNA
gggtttaggaggtgta

SEQ ID NO: 595 - RNA, 16 nt, Synthetic construct
  source 1..16: mol_type = other RNA
ggtttaggaggtgtga

SEQ ID NO: 596 - RNA, 16 nt, Synthetic construct
  source 1..16: mol_type = other RNA
tttaggaggtgtgtga

SEQ ID NO: 597 - RNA, 16 nt, Synthetic construct
  source 1..16: mol_type = other RNA
ttaggaggtgtgtgta

SEQ ID NO: 598 - RNA, 16 nt, Synthetic construct
  source 1..16: mol_type = other RNA
taggaggtgtgtgtta

SEQ ID NO: 599 - RNA, 16 nt, Synthetic construct
  source 1..16: mol_type = other RNA
ggaggtgtgtgtttta

SEQ ID NO: 600 - RNA, 16 nt, Synthetic construct
  source 1..16: mol_type = other RNA
gaggtgtgtgttttta

SEQ ID NO: 601 - RNA, 16 nt, Synthetic construct
  source 1..16: mol_type = other RNA
aggtgtgtgtttttga

SEQ ID NO: 602 - RNA, 16 nt, Synthetic construct
  source 1..16: mol_type = other RNA
tgtttttcccacccta

SEQ ID NO: 603 - RNA, 16 nt, Synthetic construct
  source 1..16: mol_type = other RNA
gagggtgaacaagaaa

SEQ ID NO: 604 - RNA, 16 nt, Synthetic construct
  source 1..16: mol_type = other RNA
agggtgaacaagaaaa

SEQ ID NO: 605 - RNA, 16 nt, Synthetic construct
  source 1..16: mol_type = other RNA
tgaacaagaaaagaca

SEQ ID NO: 606 - RNA, 16 nt, Synthetic construct
  source 1..16: mol_type = other RNA
acaagaaaagacctga

SEQ ID NO: 607 - RNA, 16 nt, Synthetic construct
  source 1..16: mol_type = other RNA
caagaaaagacctgaa

SEQ ID NO: 608 - RNA, 16 nt, Synthetic construct
  source 1..16: mol_type = other RNA
aagaaaagacctgata

SEQ ID NO: 609 - RNA, 16 nt, Synthetic construct
  source 1..16: mol_type = other RNA
gaaaagacctgataaa

SEQ ID NO: 610 - RNA, 16 nt, Synthetic construct
  source 1..16: mol_type = other RNA
tgataaagattaacca

SEQ ID NO: 611 - RNA, 16 nt, Synthetic construct
  source 1..16: mol_type = other RNA
gataaagattaaccaa

SEQ ID NO: 612 - RNA, 16 nt, Synthetic construct
  source 1..16: mol_type = other RNA
ataaagattaaccaga

SEQ ID NO: 613 - RNA, 16 nt, Synthetic construct
  source 1..16: mol_type = other RNA
aaagattaaccagaaa

SEQ ID NO: 614 - RNA, 16 nt, Synthetic construct
  source 1..16: mol_type = other RNA
aagattaaccagaaga

SEQ ID NO: 615 - RNA, 16 nt, Synthetic construct
  source 1..16: mol_type = other RNA
accagaagaaaacaaa

SEQ ID NO: 616 - RNA, 16 nt, Synthetic construct
  source 1..16: mol_type = other RNA
ccagaagaaaacaaga

SEQ ID NO: 617 - RNA, 16 nt, Synthetic construct
  source 1..16: mol_type = other RNA
cagaagaaaacaagga

SEQ ID NO: 618 - RNA, 16 nt, Synthetic construct
  source 1..16: mol_type = other RNA
gagggaaacaaccgca

SEQ ID NO: 619 - RNA, 16 nt, Synthetic construct
  source 1..16: mol_type = other RNA
agggaaacaaccgcaa

SEQ ID NO: 620 - RNA, 16 nt, Synthetic construct
  source 1..16: mol_type = other RNA
gaaacaaccgcagcca

SEQ ID NO: 621 - RNA, 16 nt, Synthetic construct
  source 1..16: mol_type = other RNA
acaaccgcagcctgta

SEQ ID NO: 622 - RNA, 16 nt, Synthetic construct
  source 1..16: mol_type = other RNA
gtcgcgcgctagggga

SEQ ID NO: 623 - RNA, 16 nt, Synthetic construct
  source 1..16: mol_type = other RNA
cggggccggggccgga

SEQ ID NO: 624 - RNA, 16 nt, Synthetic construct
  source 1..16: mol_type = other RNA
ggggccggggccggga

SEQ ID NO: 625 - RNA, 16 nt, Synthetic construct
  source 1..16: mol_type = other RNA
gggccggggccgggga

SEQ ID NO: 626 - RNA, 16 nt, Synthetic construct
  source 1..16: mol_type = other RNA
ggccggggccggggca

SEQ ID NO: 627 - RNA, 16 nt, Synthetic construct
  source 1..16: mol_type = other RNA
gccggggccggggcca

SEQ ID NO: 628 - RNA, 16 nt, Synthetic construct
  source 1..16: mol_type = other RNA
ccggggccggggccga

SEQ ID NO: 629 - RNA, 16 nt, Synthetic construct
  source 1..16: mol_type = other RNA
agctcgacgcatttta

SEQ ID NO: 630 - RNA, 16 nt, Synthetic construct
  source 1..16: mol_type = other RNA
gctcgacgcattttta

SEQ ID NO: 631 - RNA, 16 nt, Synthetic construct
  source 1..16: mol_type = other RNA
gcatttttactttcca

SEQ ID NO: 632 - RNA, 16 nt, Synthetic construct
  source 1..16: mol_type = other RNA
atttttactttcccta

SEQ ID NO: 633 - RNA, 16 nt, Synthetic construct
  source 1..16: mol_type = other RNA
tctcatttctctgaca

SEQ ID NO: 634 - RNA, 16 nt, Synthetic construct
  source 1..16: mol_type = other RNA
catttctctgaccgaa

SEQ ID NO: 635 - RNA, 16 nt, Synthetic construct
  source 1..16: mol_type = other RNA
atttctctgaccgaaa

SEQ ID NO: 636 - RNA, 16 nt, Synthetic construct
  source 1..16: mol_type = other RNA
tgaccgaagctgggta

SEQ ID NO: 637 - RNA, 16 nt, Synthetic construct
  source 1..16: mol_type = other RNA
gcctctagcgactgga

SEQ ID NO: 638 - RNA, 16 nt, Synthetic construct
  source 1..16: mol_type = other RNA
cctctagcgactggta

SEQ ID NO: 639 - RNA, 16 nt, Synthetic construct
  source 1..16: mol_type = other RNA
tctagcgactggtgga

SEQ ID NO: 640 - RNA, 16 nt, Synthetic construct
  source 1..16: mol_type = other RNA
ctagcgactggtggaa

SEQ ID NO: 641 - RNA, 16 nt, Synthetic construct
  source 1..16: mol_type = other RNA
gccctcagtacccgaa

SEQ ID NO: 642 - RNA, 16 nt, Synthetic construct
  source 1..16: mol_type = other RNA
cgcctgcacaatttca

SEQ ID NO: 643 - RNA, 16 nt, Synthetic construct
  source 1..16: mol_type = other RNA
gcacaatttcagccca

SEQ ID NO: 644 - RNA, 16 nt, Synthetic construct
  source 1..16: mol_type = other RNA
agagtggtgatgacta

SEQ ID NO: 645 - RNA, 16 nt, Synthetic construct
  source 1..16: mol_type = other RNA
agtggtgatgacttga

SEQ ID NO: 646 - RNA, 16 nt, Synthetic construct
  source 1..16: mol_type = other RNA
tgacttgcatatgaga

SEQ ID NO: 647 - RNA, 16 nt, Synthetic construct
  source 1..16: mol_type = other RNA
acttgcatatgaggga

SEQ ID NO: 648 - RNA, 16 nt, Synthetic construct
  source 1..16: mol_type = other RNA
cttgcatatgagggca

SEQ ID NO: 649 - RNA, 16 nt, Synthetic construct
  source 1..16: mol_type = other RNA
atatgagggcagcaaa

SEQ ID NO: 650 - RNA, 16 nt, Synthetic construct
  source 1..16: mol_type = other RNA
agggcagcaatgcaaa

SEQ ID NO: 651 - RNA, 16 nt, Synthetic construct
  source 1..16: mol_type = other RNA
gcagcaatgcaagtca

SEQ ID NO: 652 - RNA, 16 nt, Synthetic construct
  source 1..16: mol_type = other RNA
caatgcaagtcggtga

SEQ ID NO: 653 - RNA, 16 nt, Synthetic construct
  source 1..16: mol_type = other RNA
acatgacctggttgca

SEQ ID NO: 654 - RNA, 16 nt, Synthetic construct
  source 1..16: mol_type = other RNA
ttgcttcacagctcca

SEQ ID NO: 655 - RNA, 16 nt, Synthetic construct
  source 1..16: mol_type = other RNA
gcttcacagctccgaa

SEQ ID NO: 656 - RNA, 16 nt, Synthetic construct
  source 1..16: mol_type = other RNA
tcacagctccgagata

SEQ ID NO: 657 - RNA, 16 nt, Synthetic construct
  source 1..16: mol_type = other RNA
gctccgagatgacaca

SEQ ID NO: 658 - RNA, 16 nt, Synthetic construct
  source 1..16: mol_type = other RNA
agatgacacagactta

SEQ ID NO: 659 - RNA, 16 nt, Synthetic construct
  source 1..16: mol_type = other RNA
gatgacacagacttga

SEQ ID NO: 660 - RNA, 16 nt, Synthetic construct
  source 1..16: mol_type = other RNA
atgacacagacttgca

SEQ ID NO: 661 - RNA, 16 nt, Synthetic construct
  source 1..16: mol_type = other RNA
acacagacttgcttaa

SEQ ID NO: 662 - RNA, 16 nt, Synthetic construct
  source 1..16: mol_type = other RNA
acagacttgcttaaaa

SEQ ID NO: 663 - RNA, 16 nt, Synthetic construct
  source 1..16: mol_type = other RNA
acttgcttaaaggaaa

SEQ ID NO: 664 - RNA, 16 nt, Synthetic construct
  source 1..16: mol_type = other RNA
ttaaaggaagtgacta

SEQ ID NO: 665 - RNA, 16 nt, Synthetic construct
  source 1..16: mol_type = other RNA
aaaggaagtgactata

SEQ ID NO: 666 - RNA, 16 nt, Synthetic construct
  source 1..16: mol_type = other RNA
ggaagtgactattgta

SEQ ID NO: 667 - RNA, 16 nt, Synthetic construct
  source 1..16: mol_type = other RNA
gaagtgactattgtga

SEQ ID NO: 668 - RNA, 16 nt, Synthetic construct
  source 1..16: mol_type = other RNA
gactattgtgacttga

SEQ ID NO: 669 - RNA, 16 nt, Synthetic construct
  source 1..16: mol_type = other RNA
tattgtgacttgggca

SEQ ID NO: 670 - RNA, 16 nt, Synthetic construct
  source 1..16: mol_type = other RNA
ttgtgacttgggcata

SEQ ID NO: 671 - RNA, 16 nt, Synthetic construct
  source 1..16: mol_type = other RNA
acttgggcatcactta

SEQ ID NO: 672 - RNA, 16 nt, Synthetic construct
  source 1..16: mol_type = other RNA
cttgggcatcacttga

SEQ ID NO: 673 - RNA, 16 nt, Synthetic construct
  source 1..16: mol_type = other RNA
ttgggcatcacttgaa

SEQ ID NO: 674 - RNA, 16 nt, Synthetic construct
  source 1..16: mol_type = other RNA
tgggcatcacttgaca

SEQ ID NO: 675 - RNA, 16 nt, Synthetic construct
  source 1..16: mol_type = other RNA
gcatcacttgactgaa

SEQ ID NO: 676 - RNA, 16 nt, Synthetic construct
  source 1..16: mol_type = other RNA
catcacttgactgata

SEQ ID NO: 677 - RNA, 16 nt, Synthetic construct
  source 1..16: mol_type = other RNA
tgactgatggtaatca

SEQ ID NO: 678 - RNA, 16 nt, Synthetic construct
  source 1..16: mol_type = other RNA
actgatggtaatcaga

SEQ ID NO: 679 - RNA, 16 nt, Synthetic construct
  source 1..16: mol_type = other RNA
tgatggtaatcagtta

SEQ ID NO: 680 - RNA, 16 nt, Synthetic construct
  source 1..16: mol_type = other RNA
gatggtaatcagttga

SEQ ID NO: 681 - RNA, 16 nt, Synthetic construct
  source 1..16: mol_type = other RNA
atggtaatcagttgta

SEQ ID NO: 682 - RNA, 16 nt, Synthetic construct
  source 1..16: mol_type = other RNA
gtaatcagttgtctaa

SEQ ID NO: 683 - RNA, 16 nt, Synthetic construct
  source 1..16: mol_type = other RNA
taatcagttgtctaaa

SEQ ID NO: 684 - RNA, 16 nt, Synthetic construct
  source 1..16: mol_type = other RNA
tcagttgtctaaagaa

SEQ ID NO: 685 - RNA, 16 nt, Synthetic construct
  source 1..16: mol_type = other RNA
ttgtctaaagaagtga

SEQ ID NO: 686 - RNA, 16 nt, Synthetic construct
  source 1..16: mol_type = other RNA
tgtctaaagaagtgca

SEQ ID NO: 687 - RNA, 16 nt, Synthetic construct
  source 1..16: mol_type = other RNA
ctaaagaagtgcacaa

SEQ ID NO: 688 - RNA, 16 nt, Synthetic construct
  source 1..16: mol_type = other RNA
taaagaagtgcacaga

SEQ ID NO: 689 - RNA, 16 nt, Synthetic construct
  source 1..16: mol_type = other RNA
aaagaagtgcacagaa

SEQ ID NO: 690 - RNA, 16 nt, Synthetic construct
  source 1..16: mol_type = other RNA
aagaagtgcacagata

SEQ ID NO: 691 - RNA, 16 nt, Synthetic construct
  source 1..16: mol_type = other RNA
agaagtgcacagatta

SEQ ID NO: 692 - RNA, 16 nt, Synthetic construct
  source 1..16: mol_type = other RNA
gaagtgcacagattaa

SEQ ID NO: 693 - RNA, 16 nt, Synthetic construct
  source 1..16: mol_type = other RNA
aagtgcacagattaca

SEQ ID NO: 694 - RNA, 16 nt, Synthetic construct
  source 1..16: mol_type = other RNA
acagattacatgtcca

SEQ ID NO: 695 - RNA, 16 nt, Synthetic construct
  source 1..16: mol_type = other RNA
agattacatgtccgta

SEQ ID NO: 696 - RNA, 16 nt, Synthetic construct
  source 1..16: mol_type = other RNA
acatgtccgtgtgcta

SEQ ID NO: 697 - RNA, 16 nt, Synthetic construct
  source 1..16: mol_type = other RNA
gctcattgggtctata

SEQ ID NO: 698 - RNA, 16 nt, Synthetic construct
  source 1..16: mol_type = other RNA
tcattgggtctatcta

SEQ ID NO: 699 - RNA, 16 nt, Synthetic construct
  source 1..16: mol_type = other RNA
gggtctatctggccga

SEQ ID NO: 700 - RNA, 16 nt, Synthetic construct
  source 1..16: mol_type = other RNA
tctggccgcgttgaaa

SEQ ID NO: 701 - RNA, 16 nt, Synthetic construct
  source 1..16: mol_type = other RNA
ttgaacaccaccagga

SEQ ID NO: 702 - RNA, 16 nt, Synthetic construct
  source 1..16: mol_type = other RNA
acaccaccaggcttta

SEQ ID NO: 703 - RNA, 16 nt, Synthetic construct
  source 1..16: mol_type = other RNA
accaccaggctttgta

SEQ ID NO: 704 - RNA, 16 nt, Synthetic construct
  source 1..16: mol_type = other RNA
ccaccaggctttgtaa

SEQ ID NO: 705 - RNA, 16 nt, Synthetic construct
  source 1..16: mol_type = other RNA
attcagaaacaggaga

SEQ ID NO: 706 - RNA, 16 nt, Synthetic construct
  source 1..16: mol_type = other RNA
acaggagggaggtcca

SEQ ID NO: 707 - RNA, 16 nt, Synthetic construct
  source 1..16: mol_type = other RNA
aggtcctgcactttca

SEQ ID NO: 708 - RNA, 16 nt, Synthetic construct
  source 1..16: mol_type = other RNA
ccctttcagatgcaaa

SEQ ID NO: 709 - RNA, 16 nt, Synthetic construct
  source 1..16: mol_type = other RNA
cctttcagatgcaata

SEQ ID NO: 710 - RNA, 16 nt, Synthetic construct
  source 1..16: mol_type = other RNA
ctttcagatgcaatca

SEQ ID NO: 711 - RNA, 16 nt, Synthetic construct
  source 1..16: mol_type = other RNA
tcagatgcaatcgaga

SEQ ID NO: 712 - RNA, 16 nt, Synthetic construct
  source 1..16: mol_type = other RNA
gatgcaatcgagatta

SEQ ID NO: 713 - RNA, 16 nt, Synthetic construct
  source 1..16: mol_type = other RNA
gcaatcgagattgtta

SEQ ID NO: 714 - RNA, 16 nt, Synthetic construct
  source 1..16: mol_type = other RNA
agattgttaggctcta

SEQ ID NO: 715 - RNA, 16 nt, Synthetic construct
  source 1..16: mol_type = other RNA
gattgttaggctctga

SEQ ID NO: 716 - RNA, 16 nt, Synthetic construct
  source 1..16: mol_type = other RNA
attgttaggctctgga

SEQ ID NO: 717 - RNA, 16 nt, Synthetic construct
  source 1..16: mol_type = other RNA
ttgttaggctctggga

SEQ ID NO: 718 - RNA, 16 nt, Synthetic construct
  source 1..16: mol_type = other RNA
tgttaggctctgggaa

SEQ ID NO: 719 - RNA, 16 nt, Synthetic construct
  source 1..16: mol_type = other RNA
aggctctgggagagta

SEQ ID NO: 720 - RNA, 16 nt, Synthetic construct
  source 1..16: mol_type = other RNA
gctctgggagagtaga

SEQ ID NO: 721 - RNA, 16 nt, Synthetic construct
  source 1..16: mol_type = other RNA
ctctgggagagtagta

SEQ ID NO: 722 - RNA, 16 nt, Synthetic construct
  source 1..16: mol_type = other RNA
tctgggagagtagtta

SEQ ID NO: 723 - RNA, 16 nt, Synthetic construct
  source 1..16: mol_type = other RNA
tgggagagtagttgca

SEQ ID NO: 724 - RNA, 16 nt, Synthetic construct
  source 1..16: mol_type = other RNA
agagtagttgcctgga

SEQ ID NO: 725 - RNA, 16 nt, Synthetic construct
  source 1..16: mol_type = other RNA
agtagttgcctggtta

SEQ ID NO: 726 - RNA, 16 nt, Synthetic construct
  source 1..16: mol_type = other RNA
tagttgcctggttgta

SEQ ID NO: 727 - RNA, 16 nt, Synthetic construct
  source 1..16: mol_type = other RNA
tggttgtggcagttga

SEQ ID NO: 728 - RNA, 16 nt, Synthetic construct
  source 1..16: mol_type = other RNA
ggttgtggcagttgga

SEQ ID NO: 729 - RNA, 16 nt, Synthetic construct
  source 1..16: mol_type = other RNA
gttgtggcagttggta

SEQ ID NO: 730 - RNA, 16 nt, Synthetic construct
  source 1..16: mol_type = other RNA
ttgtggcagttggtaa

SEQ ID NO: 731 - RNA, 16 nt, Synthetic construct
  source 1..16: mol_type = other RNA
tgtggcagttggtaaa

SEQ ID NO: 732 - RNA, 16 nt, Synthetic construct
  source 1..16: mol_type = other RNA
gtggcagttggtaaaa

SEQ ID NO: 733 - RNA, 16 nt, Synthetic construct
  source 1..16: mol_type = other RNA
gcagttggtaaattta

SEQ ID NO: 734 - RNA, 16 nt, Synthetic construct
  source 1..16: mol_type = other RNA
cagttggtaaatttca

SEQ ID NO: 735 - RNA, 16 nt, Synthetic construct
  source 1..16: mol_type = other RNA
agttggtaaatttcta

SEQ ID NO: 736 - RNA, 16 nt, Synthetic construct
  source 1..16: mol_type = other RNA
gttggtaaatttctaa

SEQ ID NO: 737 - RNA, 16 nt, Synthetic construct
  source 1..16: mol_type = other RNA
ttggtaaatttctata

SEQ ID NO: 738 - RNA, 16 nt, Synthetic construct
  source 1..16: mol_type = other RNA
tggtaaatttctatta

SEQ ID NO: 739 - RNA, 16 nt, Synthetic construct
  source 1..16: mol_type = other RNA
ggtaaatttctattca

SEQ ID NO: 740 - RNA, 16 nt, Synthetic construct
  source 1..16: mol_type = other RNA
tcaaacagttgccata

SEQ ID NO: 741 - RNA, 16 nt, Synthetic construct
  source 1..16: mol_type = other RNA
aaacagttgccatgca

SEQ ID NO: 742 - RNA, 16 nt, Synthetic construct
  source 1..16: mol_type = other RNA
aacagttgccatgcaa

SEQ ID NO: 743 - RNA, 16 nt, Synthetic construct
  source 1..16: mol_type = other RNA
acagttgccatgcaca

SEQ ID NO: 744 - RNA, 16 nt, Synthetic construct
  source 1..16: mol_type = other RNA
gccatgcaccagttga

SEQ ID NO: 745 - RNA, 16 nt, Synthetic construct
  source 1..16: mol_type = other RNA
ccatgcaccagttgta

SEQ ID NO: 746 - RNA, 16 nt, Synthetic construct
  source 1..16: mol_type = other RNA
atgcaccagttgttca

SEQ ID NO: 747 - RNA, 16 nt, Synthetic construct
  source 1..16: mol_type = other RNA
tgcaccagttgttcaa

SEQ ID NO: 748 - RNA, 16 nt, Synthetic construct
  source 1..16: mol_type = other RNA
gcaccagttgttcaca

SEQ ID NO: 749 - RNA, 16 nt, Synthetic construct
  source 1..16: mol_type = other RNA
ccagttgttcacaaca

SEQ ID NO: 750 - RNA, 16 nt, Synthetic construct
  source 1..16: mol_type = other RNA
tcacaacaagggtaca

SEQ ID NO: 751 - RNA, 16 nt, Synthetic construct
  source 1..16: mol_type = other RNA
aacaagggtacgtaaa

SEQ ID NO: 752 - RNA, 16 nt, Synthetic construct
  source 1..16: mol_type = other RNA
acaagggtacgtaata

SEQ ID NO: 753 - RNA, 16 nt, Synthetic construct
  source 1..16: mol_type = other RNA
caagggtacgtaatca

SEQ ID NO: 754 - RNA, 16 nt, Synthetic construct
  source 1..16: mol_type = other RNA
gtacgtaatctgtcta

SEQ ID NO: 755 - RNA, 16 nt, Synthetic construct
  source 1..16: mol_type = other RNA
acgtaatctgtctgga

SEQ ID NO: 756 - RNA, 16 nt, Synthetic construct
  source 1..16: mol_type = other RNA
taatctgtctggcata

SEQ ID NO: 757 - RNA, 16 nt, Synthetic construct
  source 1..16: mol_type = other RNA
aatctgtctggcatta

SEQ ID NO: 758 - RNA, 16 nt, Synthetic construct
  source 1..16: mol_type = other RNA
tctgtctggcattaca

SEQ ID NO: 759 - RNA, 16 nt, Synthetic construct
  source 1..16: mol_type = other RNA
tctggcattacttcta

SEQ ID NO: 760 - RNA, 16 nt, Synthetic construct
  source 1..16: mol_type = other RNA
tggcattacttctaca

SEQ ID NO: 761 - RNA, 16 nt, Synthetic construct
  source 1..16: mol_type = other RNA
cattacttctacttta

SEQ ID NO: 762 - RNA, 16 nt, Synthetic construct
  source 1..16: mol_type = other RNA
attacttctactttta

SEQ ID NO: 763 - RNA, 16 nt, Synthetic construct
  source 1..16: mol_type = other RNA
ttacttctacttttga

SEQ ID NO: 764 - RNA, 16 nt, Synthetic construct
  source 1..16: mol_type = other RNA
ttttgtacaaaggata

SEQ ID NO: 765 - RNA, 16 nt, Synthetic construct
  source 1..16: mol_type = other RNA
tttgtacaaaggatca

SEQ ID NO: 766 - RNA, 16 nt, Synthetic construct
  source 1..16: mol_type = other RNA
ttgtacaaaggatcaa

SEQ ID NO: 767 - RNA, 16 nt, Synthetic construct
  source 1..16: mol_type = other RNA
tgtacaaaggatcaaa

SEQ ID NO: 768 - RNA, 16 nt, Synthetic construct
  source 1..16: mol_type = other RNA
gtacaaaggatcaaaa

SEQ ID NO: 769 - RNA, 21 nt, Synthetic construct
  source 1..21: mol_type = other RNA
tctgtcttggcaacagctgga

SEQ ID NO: 770 - RNA, 21 nt, Synthetic construct
  source 1..21: mol_type = other RNA
tctctgtcttggcaacagctg

SEQ ID NO: 771 - RNA, 21 nt, Synthetic construct
  source 1..21: mol_type = other RNA
tatctctgtcttggcaacagc

SEQ ID NO: 772 - RNA, 21 nt, Synthetic construct
  source 1..21: mol_type = other RNA
taatctctgtcttggcaacag

SEQ ID NO: 773 - RNA, 21 nt, Synthetic construct
  source 1..21: mol_type = other RNA
tcaatctctgtcttggcaaca

SEQ ID NO: 774 - RNA, 21 nt, Synthetic construct
  source 1..21: mol_type = other RNA
tgcaatctctgtcttggcaac

SEQ ID NO: 775 - RNA, 21 nt, Synthetic construct
  source 1..21: mol_type = other RNA
tagcaatctctgtcttggcaa

SEQ ID NO: 776 - RNA, 21 nt, Synthetic construct
  source 1..21: mol_type = other RNA
ttaaagcaatctctgtcttgg

SEQ ID NO: 777 - RNA, 21 nt, Synthetic construct
  source 1..21: mol_type = other RNA
tccacttaaagcaatctctgt

SEQ ID NO: 778 - RNA, 21 nt, Synthetic construct
  source 1..21: mol_type = other RNA
tagctgctaataaaggtgatt

SEQ ID NO: 779 - RNA, 21 nt, Synthetic construct
  source 1..21: mol_type = other RNA
ttaggaccaagaatattgtcc

SEQ ID NO: 780 - RNA, 21 nt, Synthetic construct
  source 1..21: mol_type = other RNA
tctaggaccaagaatattgtc

SEQ ID NO: 781 - RNA, 21 nt, Synthetic construct
  source 1..21: mol_type = other RNA
tctctaggaccaagaatattg

SEQ ID NO: 782 - RNA, 21 nt, Synthetic construct
  source 1..21: mol_type = other RNA
ttactctaggaccaagaatat

SEQ ID NO: 783 - RNA, 21 nt, Synthetic construct
  source 1..21: mol_type = other RNA
tccttactctaggaccaagaa

SEQ ID NO: 784 - RNA, 21 nt, Synthetic construct
  source 1..21: mol_type = other RNA
tgccttactctaggaccaaga

SEQ ID NO: 785 - RNA, 21 nt, Synthetic construct
  source 1..21: mol_type = other RNA
ttgccttactctaggaccaag

SEQ ID NO: 786 - RNA, 21 nt, Synthetic construct
  source 1..21: mol_type = other RNA
ttctttggagcccaaatgtgc

SEQ ID NO: 787 - RNA, 21 nt, Synthetic construct
  source 1..21: mol_type = other RNA
tgtctttggagcccaaatgtg

SEQ ID NO: 788 - RNA, 21 nt, Synthetic construct
  source 1..21: mol_type = other RNA
ttgtctttggagcccaaatgt

SEQ ID NO: 789 - RNA, 21 nt, Synthetic construct
  source 1..21: mol_type = other RNA
tctgtctttggagcccaaatg

SEQ ID NO: 790 - RNA, 21 nt, Synthetic construct
  source 1..21: mol_type = other RNA
ttggcaagaaaagttatttct

SEQ ID NO: 791 - RNA, 21 nt, Synthetic construct
  source 1..21: mol_type = other RNA
tggttggcaagaaaagttatt

SEQ ID NO: 792 - RNA, 21 nt, Synthetic construct
  source 1..21: mol_type = other RNA
ttggttggcaagaaaagttat

SEQ ID NO: 793 - RNA, 21 nt, Synthetic construct
  source 1..21: mol_type = other RNA
tgtggttggcaagaaaagtta

SEQ ID NO: 794 - RNA, 21 nt, Synthetic construct
  source 1..21: mol_type = other RNA
tgagtgtggttggcaagaaaa

SEQ ID NO: 795 - RNA, 21 nt, Synthetic construct
  source 1..21: mol_type = other RNA
tagagtgtggttggcaagaaa

SEQ ID NO: 796 - RNA, 21 nt, Synthetic construct
  source 1..21: mol_type = other RNA
ttagagtgtggttggcaagaa

SEQ ID NO: 797 - RNA, 21 nt, Synthetic construct
  source 1..21: mol_type = other RNA
tttagagtgtggttggcaaga

SEQ ID NO: 798 - RNA, 21 nt, Synthetic construct
  source 1..21: mol_type = other RNA
ttttagagtgtggttggcaag

SEQ ID NO: 799 - RNA, 21 nt, Synthetic construct
  source 1..21: mol_type = other RNA
tatttagagtgtggttggcaa

SEQ ID NO: 800 - RNA, 21 nt, Synthetic construct
  source 1..21: mol_type = other RNA
tcatttagagtgtggttggca

SEQ ID NO: 801 - RNA, 21 nt, Synthetic construct
  source 1..21: mol_type = other RNA
tctccatttagagtgtggttg

SEQ ID NO: 802 - RNA, 21 nt, Synthetic construct
  source 1..21: mol_type = other RNA
ttctctgcatttcgaaggatt

SEQ ID NO: 803 - RNA, 21 nt, Synthetic construct
  source 1..21: mol_type = other RNA
tcactctctgcatttcgaagg

SEQ ID NO: 804 - RNA, 21 nt, Synthetic construct
  source 1..21: mol_type = other RNA
ttacatctatagcaccactct

SEQ ID NO: 805 - RNA, 21 nt, Synthetic construct
  source 1..21: mol_type = other RNA
tttacatctatagcaccactc

SEQ ID NO: 806 - RNA, 21 nt, Synthetic construct
  source 1..21: mol_type = other RNA
tactttacatctatagcacca

SEQ ID NO: 807 - RNA, 21 nt, Synthetic construct
  source 1..21: mol_type = other RNA
taaactttacatctatagcac

SEQ ID NO: 808 - RNA, 21 nt, Synthetic construct
  source 1..21: mol_type = other RNA
tcccttttcagacaagacaaa

SEQ ID NO: 809 - RNA, 21 nt, Synthetic construct
  source 1..21: mol_type = other RNA
tcactcccttttcagacaaga

SEQ ID NO: 810 - RNA, 21 nt, Synthetic construct
  source 1..21: mol_type = other RNA
ttaatcactcccttttcagac

SEQ ID NO: 811 - RNA, 21 nt, Synthetic construct
  source 1..21: mol_type = other RNA
tataatcactcccttttcaga

SEQ ID NO: 812 - RNA, 21 nt, Synthetic construct
  source 1..21: mol_type = other RNA
taacaataatcactccctttt

SEQ ID NO: 813 - RNA, 21 nt, Synthetic construct
  source 1..21: mol_type = other RNA
tttccagtttccatcaaagat

SEQ ID NO: 814 - RNA, 21 nt, Synthetic construct
  source 1..21: mol_type = other RNA
ttctgtctgtggaagtataat

SEQ ID NO: 815 - RNA, 21 nt, Synthetic construct
  source 1..21: mol_type = other RNA
tttctgtctgtggaagtataa

SEQ ID NO: 816 - RNA, 21 nt, Synthetic construct
  source 1..21: mol_type = other RNA
taagttctgtctgtggaagta

SEQ ID NO: 817 - RNA, 21 nt, Synthetic construct
  source 1..21: mol_type = other RNA
tgaaactaagttctgtctgtg

SEQ ID NO: 818 - RNA, 21 nt, Synthetic construct
  source 1..21: mol_type = other RNA
tgaagtgggaggtagaaacta

SEQ ID NO: 819 - RNA, 21 nt, Synthetic construct
  source 1..21: mol_type = other RNA
ttgaagtgggaggtagaaact

SEQ ID NO: 820 - RNA, 21 nt, Synthetic construct
  source 1..21: mol_type = other RNA
ttcctttccggattatatgtg

SEQ ID NO: 821 - RNA, 21 nt, Synthetic construct
  source 1..21: mol_type = other RNA
tttcttcctttccggattata

SEQ ID NO: 822 - RNA, 21 nt, Synthetic construct
  source 1..21: mol_type = other RNA
tatattcttcctttccggatt

SEQ ID NO: 823 - RNA, 21 nt, Synthetic construct
  source 1..21: mol_type = other RNA
tccatattcttcctttccgga

SEQ ID NO: 824 - RNA, 21 nt, Synthetic construct
  source 1..21: mol_type = other RNA
tatgcatccatattcttcctt

SEQ ID NO: 825 - RNA, 21 nt, Synthetic construct
  source 1..21: mol_type = other RNA
tcttatgcatccatattcttc

SEQ ID NO: 826 - RNA, 21 nt, Synthetic construct
  source 1..21: mol_type = other RNA
ttccttatgcatccatattct

SEQ ID NO: 827 - RNA, 21 nt, Synthetic construct
  source 1..21: mol_type = other RNA
tttccttatgcatccatattc

SEQ ID NO: 828 - RNA, 21 nt, Synthetic construct
  source 1..21: mol_type = other RNA
ttttccttatgcatccatatt

SEQ ID NO: 829 - RNA, 21 nt, Synthetic construct
  source 1..21: mol_type = other RNA
tgtctttccttatgcatccat

SEQ ID NO: 830 - RNA, 21 nt, Synthetic construct
  source 1..21: mol_type = other RNA
ttgtctttccttatgcatcca

SEQ ID NO: 831 - RNA, 21 nt, Synthetic construct
  source 1..21: mol_type = other RNA
tttgtctttccttatgcatcc

SEQ ID NO: 832 - RNA, 21 nt, Synthetic construct
  source 1..21: mol_type = other RNA
tgacattttcttgtctttcct

SEQ ID NO: 833 - RNA, 21 nt, Synthetic construct
  source 1..21: mol_type = other RNA
ttctggacattttcttgtctt

SEQ ID NO: 834 - RNA, 21 nt, Synthetic construct
  source 1..21: mol_type = other RNA
ttctctgtgccttctaagata

SEQ ID NO: 835 - RNA, 21 nt, Synthetic construct
  source 1..21: mol_type = other RNA
tcctgatcttccattctctct

SEQ ID NO: 836 - RNA, 21 nt, Synthetic construct
  source 1..21: mol_type = other RNA
tgaccctgatcttccattctc

SEQ ID NO: 837 - RNA, 21 nt, Synthetic construct
  source 1..21: mol_type = other RNA
ttgaccctgatcttccattct

SEQ ID NO: 838 - RNA, 21 nt, Synthetic construct
  source 1..21: mol_type = other RNA
tctgaccctgatcttccattc

SEQ ID NO: 839 - RNA, 21 nt, Synthetic construct
  source 1..21: mol_type = other RNA
ttctgaccctgatcttccatt

SEQ ID NO: 840 - RNA, 21 nt, Synthetic construct
  source 1..21: mol_type = other RNA
tctctgaccctgatcttccat

SEQ ID NO: 841 - RNA, 21 nt, Synthetic construct
  source 1..21: mol_type = other RNA
tactctgaccctgatcttcca

SEQ ID NO: 842 - RNA, 21 nt, Synthetic construct
  source 1..21: mol_type = other RNA
ttactctgaccctgatcttcc

SEQ ID NO: 843 - RNA, 21 nt, Synthetic construct
  source 1..21: mol_type = other RNA
tatactctgaccctgatcttc

SEQ ID NO: 844 - RNA, 21 nt, Synthetic construct
  source 1..21: mol_type = other RNA
tataatactctgaccctgatc

SEQ ID NO: 845 - RNA, 21 nt, Synthetic construct
  source 1..21: mol_type = other RNA
tggaataatactctgaccctg

SEQ ID NO: 846 - RNA, 21 nt, Synthetic construct
  source 1..21: mol_type = other RNA
tttacaggaatcacttctcca

SEQ ID NO: 847 - RNA, 21 nt, Synthetic construct
  source 1..21: mol_type = other RNA
ttgaaagcagttccattacag

SEQ ID NO: 848 - RNA, 21 nt, Synthetic construct
  source 1..21: mol_type = other RNA
tatagatgaaagcagttccat

SEQ ID NO: 849 - RNA, 21 nt, Synthetic construct
  source 1..21: mol_type = other RNA
ttgtgtgatttcatagatgaa

SEQ ID NO: 850 - RNA, 21 nt, Synthetic construct
  source 1..21: mol_type = other RNA
taacactgtgtgatttcatag

SEQ ID NO: 851 - RNA, 21 nt, Synthetic construct
  source 1..21: mol_type = other RNA
tctatatctatttcttcagga

SEQ ID NO: 852 - RNA, 21 nt, Synthetic construct
  source 1..21: mol_type = other RNA
tctgtatcagctatatctatt

SEQ ID NO: 853 - RNA, 21 nt, Synthetic construct
  source 1..21: mol_type = other RNA
tatcattgagtactgtatcag

SEQ ID NO: 854 - RNA, 21 nt, Synthetic construct
  source 1..21: mol_type = other RNA
tcattgagaagaaagccttca

SEQ ID NO: 855 - RNA, 21 nt, Synthetic construct
  source 1..21: mol_type = other RNA
tgcattgagaagaaagccttc

SEQ ID NO: 856 - RNA, 21 nt, Synthetic construct
  source 1..21: mol_type = other RNA
tggcattgagaagaaagcctt

SEQ ID NO: 857 - RNA, 21 nt, Synthetic construct
  source 1..21: mol_type = other RNA
tgagctgatggcattgagaag

SEQ ID NO: 858 - RNA, 21 nt, Synthetic construct
  source 1..21: mol_type = other RNA
ttgagctgatggcattgagaa

SEQ ID NO: 859 - RNA, 21 nt, Synthetic construct
  source 1..21: mol_type = other RNA
tgtgagctgatggcattgaga

SEQ ID NO: 860 - RNA, 21 nt, Synthetic construct
  source 1..21: mol_type = other RNA
tgtgtgagctgatggcattga

SEQ ID NO: 861 - RNA, 21 nt, Synthetic construct
  source 1..21: mol_type = other RNA
tacggaacagccacaggtttg

SEQ ID NO: 862 - RNA, 21 nt, Synthetic construct
  source 1..21: mol_type = other RNA
tacaacggaacagccacaggt

SEQ ID NO: 863 - RNA, 21 nt, Synthetic construct
  source 1..21: mol_type = other RNA
tctacaacggaacagccacag

SEQ ID NO: 864 - RNA, 21 nt, Synthetic construct
  source 1..21: mol_type = other RNA
tgcactgctacctactacaac

SEQ ID NO: 865 - RNA, 21 nt, Synthetic construct
  source 1..21: mol_type = other RNA
tactttctctgcactgctacc

SEQ ID NO: 866 - RNA, 21 nt, Synthetic construct
  source 1..21: mol_type = other RNA
ttttactttctctgcactgct

SEQ ID NO: 867 - RNA, 21 nt, Synthetic construct
  source 1..21: mol_type = other RNA
tatttactttctctgcactgc

SEQ ID NO: 868 - RNA, 21 nt, Synthetic construct
  source 1..21: mol_type = other RNA
tatcttatttactttctctgc

SEQ ID NO: 869 - RNA, 21 nt, Synthetic construct
  source 1..21: mol_type = other RNA
tggcataatgttctgactatc

SEQ ID NO: 870 - RNA, 21 nt, Synthetic construct
  source 1..21: mol_type = other RNA
ttaaatgatgattctgcttca

SEQ ID NO: 871 - RNA, 21 nt, Synthetic construct
  source 1..21: mol_type = other RNA
tttaaatgatgattctgcttc

SEQ ID NO: 872 - RNA, 21 nt, Synthetic construct
  source 1..21: mol_type = other RNA
tccagttgaatcctttagcag

SEQ ID NO: 873 - RNA, 21 nt, Synthetic construct
  source 1..21: mol_type = other RNA
tgcatacatgacttgccggaa

SEQ ID NO: 874 - RNA, 21 nt, Synthetic construct
  source 1..21: mol_type = other RNA
tgtgtgtggtgggatatggag

SEQ ID NO: 875 - RNA, 21 nt, Synthetic construct
  source 1..21: mol_type = other RNA
tatgttcatgacagggtggca

SEQ ID NO: 876 - RNA, 21 nt, Synthetic construct
  source 1..21: mol_type = other RNA
ttatgttcatgacagggtggc

SEQ ID NO: 877 - RNA, 21 nt, Synthetic construct
  source 1..21: mol_type = other RNA
ttaaatatgttcatgacaggg

SEQ ID NO: 878 - RNA, 21 nt, Synthetic construct
  source 1..21: mol_type = other RNA
ttgtatctacgctgattataa

SEQ ID NO: 879 - RNA, 21 nt, Synthetic construct
  source 1..21: mol_type = other RNA
tctcatgtatctacgctgatt

SEQ ID NO: 880 - RNA, 21 nt, Synthetic construct
  source 1..21: mol_type = other RNA
tatgatcgtatcctgagccat

SEQ ID NO: 881 - RNA, 21 nt, Synthetic construct
  source 1..21: mol_type = other RNA
tttcgtcagtgtagatgatcg

SEQ ID NO: 882 - RNA, 21 nt, Synthetic construct
  source 1..21: mol_type = other RNA
tcaggagtaaagctttcgtca

SEQ ID NO: 883 - RNA, 21 nt, Synthetic construct
  source 1..21: mol_type = other RNA
ttattcaaatcaggagtaaag

SEQ ID NO: 884 - RNA, 21 nt, Synthetic construct
  source 1..21: mol_type = other RNA
tctgtgtaagacatcttgaaa

SEQ ID NO: 885 - RNA, 21 nt, Synthetic construct
  source 1..21: mol_type = other RNA
tagtgtctctgtgtaagacat

SEQ ID NO: 886 - RNA, 21 nt, Synthetic construct
  source 1..21: mol_type = other RNA
tgagtgtctctgtgtaagaca

SEQ ID NO: 887 - RNA, 21 nt, Synthetic construct
  source 1..21: mol_type = other RNA
tagagtgtctctgtgtaagac

SEQ ID NO: 888 - RNA, 21 nt, Synthetic construct
  source 1..21: mol_type = other RNA
ttagagtgtctctgtgtaaga

SEQ ID NO: 889 - RNA, 21 nt, Synthetic construct
  source 1..21: mol_type = other RNA
tctagagtgtctctgtgtaag

SEQ ID NO: 890 - RNA, 21 nt, Synthetic construct
  source 1..21: mol_type = other RNA
tactagagtgtctctgtgtaa

SEQ ID NO: 891 - RNA, 21 nt, Synthetic construct
  source 1..21: mol_type = other RNA
tcactagagtgtctctgtgta

SEQ ID NO: 892 - RNA, 21 nt, Synthetic construct
  source 1..21: mol_type = other RNA
ttcactagagtgtctctgtgt

SEQ ID NO: 893 - RNA, 21 nt, Synthetic construct
  source 1..21: mol_type = other RNA
tttcactagagtgtctctgtg

SEQ ID NO: 894 - RNA, 21 nt, Synthetic construct
  source 1..21: mol_type = other RNA
ttttcactagagtgtctctgt

SEQ ID NO: 895 - RNA, 21 nt, Synthetic construct
  source 1..21: mol_type = other RNA
tgtgcaaggaaagtacttctg

SEQ ID NO: 896 - RNA, 21 nt, Synthetic construct
  source 1..21: mol_type = other RNA
taactgtgcaaggaaagtact

SEQ ID NO: 897 - RNA, 21 nt, Synthetic construct
  source 1..21: mol_type = other RNA
ttcaaggcttttctgtgaagg

SEQ ID NO: 898 - RNA, 21 nt, Synthetic construct
  source 1..21: mol_type = other RNA
tagtgtcaaggcttttctgtg

SEQ ID NO: 899 - RNA, 21 nt, Synthetic construct
  source 1..21: mol_type = other RNA
ttagtgtcaaggcttttctgt

SEQ ID NO: 900 - RNA, 21 nt, Synthetic construct
  source 1..21: mol_type = other RNA
tttagtgtcaaggcttttctg

SEQ ID NO: 901 - RNA, 21 nt, Synthetic construct
  source 1..21: mol_type = other RNA
tttttcccttctgcgtatcgt

SEQ ID NO: 902 - RNA, 21 nt, Synthetic construct
  source 1..21: mol_type = other RNA
ttttttcccttctgcgtatcg

SEQ ID NO: 903 - RNA, 21 nt, Synthetic construct
  source 1..21: mol_type = other RNA
ttctgctgttaaatcaaggtc

SEQ ID NO: 904 - RNA, 21 nt, Synthetic construct
  source 1..21: mol_type = other RNA
tctctgctgttaaatcaaggt

SEQ ID NO: 905 - RNA, 21 nt, Synthetic construct
  source 1..21: mol_type = other RNA
tcctctgctgttaaatcaagg

SEQ ID NO: 906 - RNA, 21 nt, Synthetic construct
  source 1..21: mol_type = other RNA
tccctctgctgttaaatcaag

SEQ ID NO: 907 - RNA, 21 nt, Synthetic construct
  source 1..21: mol_type = other RNA
tgccctctgctgttaaatcaa

SEQ ID NO: 908 - RNA, 21 nt, Synthetic construct
  source 1..21: mol_type = other RNA
tcgccctctgctgttaaatca

SEQ ID NO: 909 - RNA, 21 nt, Synthetic construct
  source 1..21: mol_type = other RNA
tatcgccctctgctgttaaat

SEQ ID NO: 910 - RNA, 21 nt, Synthetic construct
  source 1..21: mol_type = other RNA
tgatcgccctctgctgttaaa

SEQ ID NO: 911 - RNA, 21 nt, Synthetic construct
  source 1..21: mol_type = other RNA
taagatcgccctctgctgtta

SEQ ID NO: 912 - RNA, 21 nt, Synthetic construct
  source 1..21: mol_type = other RNA
ttaagatcgccctctgctgtt

SEQ ID NO: 913 - RNA, 21 nt, Synthetic construct
  source 1..21: mol_type = other RNA
tgttaagatcgccctctgctg

SEQ ID NO: 914 - RNA, 21 nt, Synthetic construct
  source 1..21: mol_type = other RNA
tttatgttaagatcgccctct

SEQ ID NO: 915 - RNA, 21 nt, Synthetic construct
  source 1..21: mol_type = other RNA
tattatgttaagatcgccctc

SEQ ID NO: 916 - RNA, 21 nt, Synthetic construct
  source 1..21: mol_type = other RNA
ttattatgttaagatcgccct

SEQ ID NO: 917 - RNA, 21 nt, Synthetic construct
  source 1..21: mol_type = other RNA
tttattatgttaagatcgccc

SEQ ID NO: 918 - RNA, 21 nt, Synthetic construct
  source 1..21: mol_type = other RNA
tccattattatgttaagatcg

SEQ ID NO: 919 - RNA, 21 nt, Synthetic construct
  source 1..21: mol_type = other RNA
tgccattattatgttaagatc

SEQ ID NO: 920 - RNA, 21 nt, Synthetic construct
  source 1..21: mol_type = other RNA
tgagccattattatgttaaga

SEQ ID NO: 921 - RNA, 21 nt, Synthetic construct
  source 1..21: mol_type = other RNA
tagagccattattatgttaag

SEQ ID NO: 922 - RNA, 21 nt, Synthetic construct
  source 1..21: mol_type = other RNA
ttaattttctcagccagagcc

SEQ ID NO: 923 - RNA, 21 nt, Synthetic construct
  source 1..21: mol_type = other RNA
ttttaattttctcagccagag

SEQ ID NO: 924 - RNA, 21 nt, Synthetic construct
  source 1..21: mol_type = other RNA
taagagtgtaggcctggttta

SEQ ID NO: 925 - RNA, 21 nt, Synthetic construct
  source 1..21: mol_type = other RNA
ttagtgtagaaaggtcttcca

SEQ ID NO: 926 - RNA, 21 nt, Synthetic construct
  source 1..21: mol_type = other RNA
tctagtgtagaaaggtcttcc

SEQ ID NO: 927 - RNA, 21 nt, Synthetic construct
  source 1..21: mol_type = other RNA
tactagtgtagaaaggtcttc

SEQ ID NO: 928 - RNA, 21 nt, Synthetic construct
  source 1..21: mol_type = other RNA
tcactagtgtagaaaggtctt

SEQ ID NO: 929 - RNA, 21 nt, Synthetic construct
  source 1..21: mol_type = other RNA
ttagaacatctcgttcttgca

SEQ ID NO: 930 - RNA, 21 nt, Synthetic construct
  source 1..21: mol_type = other RNA
tataggcttattaagttacac

SEQ ID NO: 931 - RNA, 21 nt, Synthetic construct
  source 1..21: mol_type = other RNA
tggaataggcttattaagtta

SEQ ID NO: 932 - RNA, 21 nt, Synthetic construct
  source 1..21: mol_type = other RNA
tgtgatggaataggcttatta

SEQ ID NO: 933 - RNA, 21 nt, Synthetic construct
  source 1..21: mol_type = other RNA
tttacttaactgcaattgctg

SEQ ID NO: 934 - RNA, 21 nt, Synthetic construct
  source 1..21: mol_type = other RNA
tgtgtaacttacttaactgca

SEQ ID NO: 935 - RNA, 21 nt, Synthetic construct
  source 1..21: mol_type = other RNA
tagaactgtagtgtaacttac

SEQ ID NO: 936 - RNA, 21 nt, Synthetic construct
  source 1..21: mol_type = other RNA
tccaatgtaatgatgcacctg

SEQ ID NO: 937 - RNA, 21 nt, Synthetic construct
  source 1..21: mol_type = other RNA
taggtctgtatcccaaaagca

SEQ ID NO: 938 - RNA, 21 nt, Synthetic construct
  source 1..21: mol_type = other RNA
ttaggtctgtatcccaaaagc

SEQ ID NO: 939 - RNA, 21 nt, Synthetic construct
  source 1..21: mol_type = other RNA
tcataggtctgtatcccaaaa

SEQ ID NO: 940 - RNA, 21 nt, Synthetic construct
  source 1..21: mol_type = other RNA
taacataggtctgtatcccaa

SEQ ID NO: 941 - RNA, 21 nt, Synthetic construct
  source 1..21: mol_type = other RNA
taaacataggtctgtatccca

SEQ ID NO: 942 - RNA, 21 nt, Synthetic construct
  source 1..21: mol_type = other RNA
tattgtaaacataggtctgta

SEQ ID NO: 943 - RNA, 21 nt, Synthetic construct
  source 1..21: mol_type = other RNA
tatattgtaaacataggtctg

SEQ ID NO: 944 - RNA, 21 nt, Synthetic construct
  source 1..21: mol_type = other RNA
tgtaaaccatgaatcatgtat

SEQ ID NO: 945 - RNA, 21 nt, Synthetic construct
  source 1..21: mol_type = other RNA
tccaaaagatagaaagtcaac

SEQ ID NO: 946 - RNA, 21 nt, Synthetic construct
  source 1..21: mol_type = other RNA
ttcactttctttctgtggaat

SEQ ID NO: 947 - RNA, 21 nt, Synthetic construct
  source 1..21: mol_type = other RNA
tattaaacatctgcttgatca

SEQ ID NO: 948 - RNA, 21 nt, Synthetic construct
  source 1..21: mol_type = other RNA
ttccaattaaacatctgcttg

SEQ ID NO: 949 - RNA, 21 nt, Synthetic construct
  source 1..21: mol_type = other RNA
tctacagactgaatcccaggg

SEQ ID NO: 950 - RNA, 21 nt, Synthetic construct
  source 1..21: mol_type = other RNA
tagacatttctacagactgaa

SEQ ID NO: 951 - RNA, 21 nt, Synthetic construct
  source 1..21: mol_type = other RNA
tctaactgtggttcaccagga

SEQ ID NO: 952 - RNA, 21 nt, Synthetic construct
  source 1..21: mol_type = other RNA
tttacagagctcaactacata

SEQ ID NO: 953 - RNA, 21 nt, Synthetic construct
  source 1..21: mol_type = other RNA
ttttacagagctcaactacat

SEQ ID NO: 954 - RNA, 21 nt, Synthetic construct
  source 1..21: mol_type = other RNA
tcttttacagagctcaactac

SEQ ID NO: 955 - RNA, 21 nt, Synthetic construct
  source 1..21: mol_type = other RNA
ttccttttacagagctcaact

SEQ ID NO: 956 - RNA, 21 nt, Synthetic construct
  source 1..21: mol_type = other RNA
tttccttttacagagctcaac

SEQ ID NO: 957 - RNA, 21 nt, Synthetic construct
  source 1..21: mol_type = other RNA
tcacatttcaatttggctcaa

SEQ ID NO: 958 - RNA, 21 nt, Synthetic construct
  source 1..21: mol_type = other RNA
taaaggcacaggaggtgcaca

SEQ ID NO: 959 - RNA, 21 nt, Synthetic construct
  source 1..21: mol_type = other RNA
tactgaccagtgaaatctgaa

SEQ ID NO: 960 - RNA, 21 nt, Synthetic construct
  source 1..21: mol_type = other RNA
taaagcaggtacatggtaaga

SEQ ID NO: 961 - RNA, 21 nt, Synthetic construct
  source 1..21: mol_type = other RNA
tcctctctttcctagcgggac

SEQ ID NO: 962 - RNA, 21 nt, Synthetic construct
  source 1..21: mol_type = other RNA
tggaacttgtcgctgtttgac

SEQ ID NO: 963 - RNA, 21 nt, Synthetic construct
  source 1..21: mol_type = other RNA
tcggaacttgtcgctgtttga

SEQ ID NO: 964 - RNA, 21 nt, Synthetic construct
  source 1..21: mol_type = other RNA
tgtgggcggaacttgtcgctg

SEQ ID NO: 965 - RNA, 21 nt, Synthetic construct
  source 1..21: mol_type = other RNA
ttacgtgggcggaacttgtcg

SEQ ID NO: 966 - RNA, 21 nt, Synthetic construct
  source 1..21: mol_type = other RNA
tttacgtgggcggaacttgtc

SEQ ID NO: 967 - RNA, 21 nt, Synthetic construct
  source 1..21: mol_type = other RNA
tcttttacgtgggcggaactt

SEQ ID NO: 968 - RNA, 21 nt, Synthetic construct
  source 1..21: mol_type = other RNA
tcatcttttacgtgggcggaa

SEQ ID NO: 969 - RNA, 21 nt, Synthetic construct
  source 1..21: mol_type = other RNA
ttcatcttttacgtgggcgga

SEQ ID NO: 970 - RNA, 21 nt, Synthetic construct
  source 1..21: mol_type = other RNA
tgtcatcttttacgtgggcgg

SEQ ID NO: 971 - RNA, 21 nt, Synthetic construct
  source 1..21: mol_type = other RNA
tcgtcatcttttacgtgggcg

SEQ ID NO: 972 - RNA, 21 nt, Synthetic construct
  source 1..21: mol_type = other RNA
taagcgtcatcttttacgtgg

SEQ ID NO: 973 - RNA, 21 nt, Synthetic construct
  source 1..21: mol_type = other RNA
tagcagggacggctgacacac

SEQ ID NO: 974 - RNA, 21 nt, Synthetic construct
  source 1..21: mol_type = other RNA
tagagaagcaaccgggcagca

SEQ ID NO: 975 - RNA, 21 nt, Synthetic construct
  source 1..21: mol_type = other RNA
tcacacctgctcttgctagac

SEQ ID NO: 976 - RNA, 21 nt, Synthetic construct
  source 1..21: mol_type = other RNA
ttaaacccacacctgctcttg

SEQ ID NO: 977 - RNA, 21 nt, Synthetic construct
  source 1..21: mol_type = other RNA
tcacctcctaaacccacacct

SEQ ID NO: 978 - RNA, 21 nt, Synthetic construct
  source 1..21: mol_type = other RNA
tacacctcctaaacccacacc

SEQ ID NO: 979 - RNA, 21 nt, Synthetic construct
  source 1..21: mol_type = other RNA
tcacacctcctaaacccacac

SEQ ID NO: 980 - RNA, 21 nt, Synthetic construct
  source 1..21: mol_type = other RNA
tcacacacctcctaaacccac

SEQ ID NO: 981 - RNA, 21 nt, Synthetic construct
  source 1..21: mol_type = other RNA
tacacacacctcctaaaccca

SEQ ID NO: 982 - RNA, 21 nt, Synthetic construct
  source 1..21: mol_type = other RNA
taacacacacctcctaaaccc

SEQ ID NO: 983 - RNA, 21 nt, Synthetic construct
  source 1..21: mol_type = other RNA
taaaacacacacctcctaaac

SEQ ID NO: 984 - RNA, 21 nt, Synthetic construct
  source 1..21: mol_type = other RNA
taaaaacacacacctcctaaa

SEQ ID NO: 985 - RNA, 21 nt, Synthetic construct
  source 1..21: mol_type = other RNA
tcaaaaacacacacctcctaa

SEQ ID NO: 986 - RNA, 21 nt, Synthetic construct
  source 1..21: mol_type = other RNA
tagggtgggaaaaacaaaaac

SEQ ID NO: 987 - RNA, 21 nt, Synthetic construct
  source 1..21: mol_type = other RNA
tttcttgttcaccctcagcga

SEQ ID NO: 988 - RNA, 21 nt, Synthetic construct
  source 1..21: mol_type = other RNA
ttttcttgttcaccctcagcg

SEQ ID NO: 989 - RNA, 21 nt, Synthetic construct
  source 1..21: mol_type = other RNA
tgtcttttcttgttcaccctc

SEQ ID NO: 990 - RNA, 21 nt, Synthetic construct
  source 1..21: mol_type = other RNA
tcaggtcttttcttgttcacc

SEQ ID NO: 991 - RNA, 21 nt, Synthetic construct
  source 1..21: mol_type = other RNA
ttcaggtcttttcttgttcac

SEQ ID NO: 992 - RNA, 21 nt, Synthetic construct
  source 1..21: mol_type = other RNA
tatcaggtcttttcttgttca

SEQ ID NO: 993 - RNA, 21 nt, Synthetic construct
  source 1..21: mol_type = other RNA
tttatcaggtcttttcttgtt

SEQ ID NO: 994 - RNA, 21 nt, Synthetic construct
  source 1..21: mol_type = other RNA
tggttaatctttatcaggtct

SEQ ID NO: 995 - RNA, 21 nt, Synthetic construct
  source 1..21: mol_type = other RNA
ttggttaatctttatcaggtc

SEQ ID NO: 996 - RNA, 21 nt, Synthetic construct
  source 1..21: mol_type = other RNA
tctggttaatctttatcaggt

SEQ ID NO: 997 - RNA, 21 nt, Synthetic construct
  source 1..21: mol_type = other RNA
tttctggttaatctttatcag

SEQ ID NO: 998 - RNA, 21 nt, Synthetic construct
  source 1..21: mol_type = other RNA
tcttctggttaatctttatca

SEQ ID NO: 999 - RNA, 21 nt, Synthetic construct
  source 1..21: mol_type = other RNA
tttgttttcttctggttaatc

SEQ ID NO: 1000 - RNA, 21 nt, Synthetic construct
  source 1..21: mol_type = other RNA
tcttgttttcttctggttaat

SEQ ID NO: 1001 - RNA, 21 nt, Synthetic construct
  source 1..21: mol_type = other RNA
tccttgttttcttctggttaa

SEQ ID NO: 1002 - RNA, 21 nt, Synthetic construct
  source 1..21: mol_type = other RNA
tgcggttgtttccctccttgt

SEQ ID NO: 1003 - RNA, 21 nt, Synthetic construct
  source 1..21: mol_type = other RNA
ttgcggttgtttccctccttg

SEQ ID NO: 1004 - RNA, 21 nt, Synthetic construct
  source 1..21: mol_type = other RNA
tggctgcggttgtttccctcc

SEQ ID NO: 1005 - RNA, 21 nt, Synthetic construct
  source 1..21: mol_type = other RNA
tacaggctgcggttgtttccc

SEQ ID NO: 1006 - RNA, 21 nt, Synthetic construct
  source 1..21: mol_type = other RNA
tcccctagcgcgcgactcctg

SEQ ID NO: 1007 - RNA, 21 nt, Synthetic construct
  source 1..21: mol_type = other RNA
tccggccccggccccggcccc

SEQ ID NO: 1008 - RNA, 21 nt, Synthetic construct
  source 1..21: mol_type = other RNA
tcccggccccggccccggccc

SEQ ID NO: 1009 - RNA, 21 nt, Synthetic construct
  source 1..21: mol_type = other RNA
tccccggccccggccccggcc

SEQ ID NO: 1010 - RNA, 21 nt, Synthetic construct
  source 1..21: mol_type = other RNA
tgccccggccccggccccggc

SEQ ID NO: 1011 - RNA, 21 nt, Synthetic construct
  source 1..21: mol_type = other RNA
tggccccggccccggccccgg

SEQ ID NO: 1012 - RNA, 21 nt, Synthetic construct
  source 1..21: mol_type = other RNA
tcggccccggccccggccccg

SEQ ID NO: 1013 - RNA, 21 nt, Synthetic construct
  source 1..21: mol_type = other RNA
taaaatgcgtcgagctctgag

SEQ ID NO: 1014 - RNA, 21 nt, Synthetic construct
  source 1..21: mol_type = other RNA
taaaaatgcgtcgagctctga

SEQ ID NO: 1015 - RNA, 21 nt, Synthetic construct
  source 1..21: mol_type = other RNA
tggaaagtaaaaatgcgtcga

SEQ ID NO: 1016 - RNA, 21 nt, Synthetic construct
  source 1..21: mol_type = other RNA
tagggaaagtaaaaatgcgtc

SEQ ID NO: 1017 - RNA, 21 nt, Synthetic construct
  source 1..21: mol_type = other RNA
tgtcagagaaatgagagggaa

SEQ ID NO: 1018 - RNA, 21 nt, Synthetic construct
  source 1..21: mol_type = other RNA
ttcggtcagagaaatgagagg

SEQ ID NO: 1019 - RNA, 21 nt, Synthetic construct
  source 1..21: mol_type = other RNA
tttcggtcagagaaatgagag

SEQ ID NO: 1020 - RNA, 21 nt, Synthetic construct
  source 1..21: mol_type = other RNA
tacccagcttcggtcagagaa

SEQ ID NO: 1021 - RNA, 21 nt, Synthetic construct
  source 1..21: mol_type = other RNA
tccagtcgctagaggcgaaag

SEQ ID NO: 1022 - RNA, 21 nt, Synthetic construct
  source 1..21: mol_type = other RNA
taccagtcgctagaggcgaaa

SEQ ID NO: 1023 - RNA, 21 nt, Synthetic construct
  source 1..21: mol_type = other RNA
tccaccagtcgctagaggcga

SEQ ID NO: 1024 - RNA, 21 nt, Synthetic construct
  source 1..21: mol_type = other RNA
ttccaccagtcgctagaggcg

SEQ ID NO: 1025 - RNA, 21 nt, Synthetic construct
  source 1..21: mol_type = other RNA
ttcgggtactgagggcgggaa

SEQ ID NO: 1026 - RNA, 21 nt, Synthetic construct
  source 1..21: mol_type = other RNA
tgaaattgtgcaggcgtctcc

SEQ ID NO: 1027 - RNA, 21 nt, Synthetic construct
  source 1..21: mol_type = other RNA
tgggctgaaattgtgcaggcg

SEQ ID NO: 1028 - RNA, 21 nt, Synthetic construct
  source 1..21: mol_type = other RNA
tagtcatcaccactctctaga

SEQ ID NO: 1029 - RNA, 21 nt, Synthetic construct
  source 1..21: mol_type = other RNA
tcaagtcatcaccactctcta

SEQ ID NO: 1030 - RNA, 21 nt, Synthetic construct
  source 1..21: mol_type = other RNA
tctcatatgcaagtcatcacc

SEQ ID NO: 1031 - RNA, 21 nt, Synthetic construct
  source 1..21: mol_type = other RNA
tccctcatatgcaagtcatca

SEQ ID NO: 1032 - RNA, 21 nt, Synthetic construct
  source 1..21: mol_type = other RNA
tgccctcatatgcaagtcatc

SEQ ID NO: 1033 - RNA, 21 nt, Synthetic construct
  source 1..21: mol_type = other RNA
tttgctgccctcatatgcaag

SEQ ID NO: 1034 - RNA, 21 nt, Synthetic construct
  source 1..21: mol_type = other RNA
tttgcattgctgccctcatat

SEQ ID NO: 1035 - RNA, 21 nt, Synthetic construct
  source 1..21: mol_type = other RNA
tgacttgcattgctgccctca

SEQ ID NO: 1036 - RNA, 21 nt, Synthetic construct
  source 1..21: mol_type = other RNA
tcaccgacttgcattgctgcc

SEQ ID NO: 1037 - RNA, 21 nt, Synthetic construct
  source 1..21: mol_type = other RNA
tgcaaccaggtcatgtcccac

SEQ ID NO: 1038 - RNA, 21 nt, Synthetic construct
  source 1..21: mol_type = other RNA
tggagctgtgaagcaaccagg

SEQ ID NO: 1039 - RNA, 21 nt, Synthetic construct
  source 1..21: mol_type = other RNA
ttcggagctgtgaagcaacca

SEQ ID NO: 1040 - RNA, 21 nt, Synthetic construct
  source 1..21: mol_type = other RNA
tatctcggagctgtgaagcaa

SEQ ID NO: 1041 - RNA, 21 nt, Synthetic construct
  source 1..21: mol_type = other RNA
tgtgtcatctcggagctgtga

SEQ ID NO: 1042 - RNA, 21 nt, Synthetic construct
  source 1..21: mol_type = other RNA
taagtctgtgtcatctcggag

SEQ ID NO: 1043 - RNA, 21 nt, Synthetic construct
  source 1..21: mol_type = other RNA
tcaagtctgtgtcatctcgga

SEQ ID NO: 1044 - RNA, 21 nt, Synthetic construct
  source 1..21: mol_type = other RNA
tgcaagtctgtgtcatctcgg

SEQ ID NO: 1045 - RNA, 21 nt, Synthetic construct
  source 1..21: mol_type = other RNA
ttaagcaagtctgtgtcatct

SEQ ID NO: 1046 - RNA, 21 nt, Synthetic construct
  source 1..21: mol_type = other RNA
ttttaagcaagtctgtgtcat

SEQ ID NO: 1047 - RNA, 21 nt, Synthetic construct
  source 1..21: mol_type = other RNA
tttcctttaagcaagtctgtg

SEQ ID NO: 1048 - RNA, 21 nt, Synthetic construct
  source 1..21: mol_type = other RNA
tagtcacttcctttaagcaag

SEQ ID NO: 1049 - RNA, 21 nt, Synthetic construct
  source 1..21: mol_type = other RNA
tatagtcacttcctttaagca

SEQ ID NO: 1050 - RNA, 21 nt, Synthetic construct
  source 1..21: mol_type = other RNA
tacaatagtcacttcctttaa

SEQ ID NO: 1051 - RNA, 21 nt, Synthetic construct
  source 1..21: mol_type = other RNA
tcacaatagtcacttccttta

SEQ ID NO: 1052 - RNA, 21 nt, Synthetic construct
  source 1..21: mol_type = other RNA
tcaagtcacaatagtcacttc

SEQ ID NO: 1053 - RNA, 21 nt, Synthetic construct
  source 1..21: mol_type = other RNA
tgcccaagtcacaatagtcac

SEQ ID NO: 1054 - RNA, 21 nt, Synthetic construct
  source 1..21: mol_type = other RNA
tatgcccaagtcacaatagtc

SEQ ID NO: 1055 - RNA, 21 nt, Synthetic construct
  source 1..21: mol_type = other RNA
taagtgatgcccaagtcacaa

SEQ ID NO: 1056 - RNA, 21 nt, Synthetic construct
  source 1..21: mol_type = other RNA
tcaagtgatgcccaagtcaca

SEQ ID NO: 1057 - RNA, 21 nt, Synthetic construct
  source 1..21: mol_type = other RNA
ttcaagtgatgcccaagtcac

SEQ ID NO: 1058 - RNA, 21 nt, Synthetic construct
  source 1..21: mol_type = other RNA
tgtcaagtgatgcccaagtca

SEQ ID NO: 1059 - RNA, 21 nt, Synthetic construct
  source 1..21: mol_type = other RNA
ttcagtcaagtgatgcccaag

SEQ ID NO: 1060 - RNA, 21 nt, Synthetic construct
  source 1..21: mol_type = other RNA
tatcagtcaagtgatgcccaa

SEQ ID NO: 1061 - RNA, 21 nt, Synthetic construct
  source 1..21: mol_type = other RNA
tgattaccatcagtcaagtga

SEQ ID NO: 1062 - RNA, 21 nt, Synthetic construct
  source 1..21: mol_type = other RNA
tctgattaccatcagtcaagt

SEQ ID NO: 1063 - RNA, 21 nt, Synthetic construct
  source 1..21: mol_type = other RNA
taactgattaccatcagtcaa

SEQ ID NO: 1064 - RNA, 21 nt, Synthetic construct
  source 1..21: mol_type = other RNA
tcaactgattaccatcagtca

SEQ ID NO: 1065 - RNA, 21 nt, Synthetic construct
  source 1..21: mol_type = other RNA
tacaactgattaccatcagtc

SEQ ID NO: 1066 - RNA, 21 nt, Synthetic construct
  source 1..21: mol_type = other RNA
ttagacaactgattaccatca

SEQ ID NO: 1067 - RNA, 21 nt, Synthetic construct
  source 1..21: mol_type = other RNA
tttagacaactgattaccatc

SEQ ID NO: 1068 - RNA, 21 nt, Synthetic construct
  source 1..21: mol_type = other RNA
ttctttagacaactgattacc

SEQ ID NO: 1069 - RNA, 21 nt, Synthetic construct
  source 1..21: mol_type = other RNA
tcacttctttagacaactgat

SEQ ID NO: 1070 - RNA, 21 nt, Synthetic construct
  source 1..21: mol_type = other RNA
tgcacttctttagacaactga

SEQ ID NO: 1071 - RNA, 21 nt, Synthetic construct
  source 1..21: mol_type = other RNA
ttgtgcacttctttagacaac

SEQ ID NO: 1072 - RNA, 21 nt, Synthetic construct
  source 1..21: mol_type = other RNA
tctgtgcacttctttagacaa

SEQ ID NO: 1073 - RNA, 21 nt, Synthetic construct
  source 1..21: mol_type = other RNA
ttctgtgcacttctttagaca

SEQ ID NO: 1074 - RNA, 21 nt, Synthetic construct
  source 1..21: mol_type = other RNA
tatctgtgcacttctttagac

SEQ ID NO: 1075 - RNA, 21 nt, Synthetic construct
  source 1..21: mol_type = other RNA
taatctgtgcacttctttaga

SEQ ID NO: 1076 - RNA, 21 nt, Synthetic construct
  source 1..21: mol_type = other RNA
ttaatctgtgcacttctttag

SEQ ID NO: 1077 - RNA, 21 nt, Synthetic construct
  source 1..21: mol_type = other RNA
tgtaatctgtgcacttcttta

SEQ ID NO: 1078 - RNA, 21 nt, Synthetic construct
  source 1..21: mol_type = other RNA
tggacatgtaatctgtgcact

SEQ ID NO: 1079 - RNA, 21 nt, Synthetic construct
  source 1..21: mol_type = other RNA
tacggacatgtaatctgtgca

SEQ ID NO: 1080 - RNA, 21 nt, Synthetic construct
  source 1..21: mol_type = other RNA
tagcacacggacatgtaatct

SEQ ID NO: 1081 - RNA, 21 nt, Synthetic construct
  source 1..21: mol_type = other RNA
tatagacccaatgagcacacg

SEQ ID NO: 1082 - RNA, 21 nt, Synthetic construct
  source 1..21: mol_type = other RNA
tagatagacccaatgagcaca

SEQ ID NO: 1083 - RNA, 21 nt, Synthetic construct
  source 1..21: mol_type = other RNA
tcggccagatagacccaatga

SEQ ID NO: 1084 - RNA, 21 nt, Synthetic construct
  source 1..21: mol_type = other RNA
tttcaacgcggccagatagac

SEQ ID NO: 1085 - RNA, 21 nt, Synthetic construct
  source 1..21: mol_type = other RNA
tcctggtggtgttcaacgcgg

SEQ ID NO: 1086 - RNA, 21 nt, Synthetic construct
  source 1..21: mol_type = other RNA
taaagcctggtggtgttcaac

SEQ ID NO: 1087 - RNA, 21 nt, Synthetic construct
  source 1..21: mol_type = other RNA
tacaaagcctggtggtgttca

SEQ ID NO: 1088 - RNA, 21 nt, Synthetic construct
  source 1..21: mol_type = other RNA
ttacaaagcctggtggtgttc

SEQ ID NO: 1089 - RNA, 21 nt, Synthetic construct
  source 1..21: mol_type = other RNA
tctcctgtttctgaatacaaa

SEQ ID NO: 1090 - RNA, 21 nt, Synthetic construct
  source 1..21: mol_type = other RNA
tggacctccctcctgtttctg

SEQ ID NO: 1091 - RNA, 21 nt, Synthetic construct
  source 1..21: mol_type = other RNA
tgaaagtgcaggacctccctc

SEQ ID NO: 1092 - RNA, 21 nt, Synthetic construct
  source 1..21: mol_type = other RNA
tttgcatctgaaagggccacc

SEQ ID NO: 1093 - RNA, 21 nt, Synthetic construct
  source 1..21: mol_type = other RNA
tattgcatctgaaagggccac

SEQ ID NO: 1094 - RNA, 21 nt, Synthetic construct
  source 1..21: mol_type = other RNA
tgattgcatctgaaagggcca

SEQ ID NO: 1095 - RNA, 21 nt, Synthetic construct
  source 1..21: mol_type = other RNA
tctcgattgcatctgaaaggg

SEQ ID NO: 1096 - RNA, 21 nt, Synthetic construct
  source 1..21: mol_type = other RNA
taatctcgattgcatctgaaa

SEQ ID NO: 1097 - RNA, 21 nt, Synthetic construct
  source 1..21: mol_type = other RNA
taacaatctcgattgcatctg

SEQ ID NO: 1098 - RNA, 21 nt, Synthetic construct
  source 1..21: mol_type = other RNA
tagagcctaacaatctcgatt

SEQ ID NO: 1099 - RNA, 21 nt, Synthetic construct
  source 1..21: mol_type = other RNA
tcagagcctaacaatctcgat

SEQ ID NO: 1100 - RNA, 21 nt, Synthetic construct
  source 1..21: mol_type = other RNA
tccagagcctaacaatctcga

SEQ ID NO: 1101 - RNA, 21 nt, Synthetic construct
  source 1..21: mol_type = other RNA
tcccagagcctaacaatctcg

SEQ ID NO: 1102 - RNA, 21 nt, Synthetic construct
  source 1..21: mol_type = other RNA
ttcccagagcctaacaatctc

SEQ ID NO: 1103 - RNA, 21 nt, Synthetic construct
  source 1..21: mol_type = other RNA
tactctcccagagcctaacaa

SEQ ID NO: 1104 - RNA, 21 nt, Synthetic construct
  source 1..21: mol_type = other RNA
tctactctcccagagcctaac

SEQ ID NO: 1105 - RNA, 21 nt, Synthetic construct
  source 1..21: mol_type = other RNA
tactactctcccagagcctaa

SEQ ID NO: 1106 - RNA, 21 nt, Synthetic construct
  source 1..21: mol_type = other RNA
taactactctcccagagccta

SEQ ID NO: 1107 - RNA, 21 nt, Synthetic construct
  source 1..21: mol_type = other RNA
tgcaactactctcccagagcc

SEQ ID NO: 1108 - RNA, 21 nt, Synthetic construct
  source 1..21: mol_type = other RNA
tccaggcaactactctcccag

SEQ ID NO: 1109 - RNA, 21 nt, Synthetic construct
  source 1..21: mol_type = other RNA
taaccaggcaactactctccc

SEQ ID NO: 1110 - RNA, 21 nt, Synthetic construct
  source 1..21: mol_type = other RNA
tacaaccaggcaactactctc

SEQ ID NO: 1111 - RNA, 21 nt, Synthetic construct
  source 1..21: mol_type = other RNA
tcaactgccacaaccaggcaa

SEQ ID NO: 1112 - RNA, 21 nt, Synthetic construct
  source 1..21: mol_type = other RNA
tccaactgccacaaccaggca

SEQ ID NO: 1113 - RNA, 21 nt, Synthetic construct
  source 1..21: mol_type = other RNA
taccaactgccacaaccaggc

SEQ ID NO: 1114 - RNA, 21 nt, Synthetic construct
  source 1..21: mol_type = other RNA
ttaccaactgccacaaccagg

SEQ ID NO: 1115 - RNA, 21 nt, Synthetic construct
  source 1..21: mol_type = other RNA
tttaccaactgccacaaccag

SEQ ID NO: 1116 - RNA, 21 nt, Synthetic construct
  source 1..21: mol_type = other RNA
ttttaccaactgccacaacca

SEQ ID NO: 1117 - RNA, 21 nt, Synthetic construct
  source 1..21: mol_type = other RNA
taaatttaccaactgccacaa

SEQ ID NO: 1118 - RNA, 21 nt, Synthetic construct
  source 1..21: mol_type = other RNA
tgaaatttaccaactgccaca

SEQ ID NO: 1119 - RNA, 21 nt, Synthetic construct
  source 1..21: mol_type = other RNA
tagaaatttaccaactgccac

SEQ ID NO: 1120 - RNA, 21 nt, Synthetic construct
  source 1..21: mol_type = other RNA
ttagaaatttaccaactgcca

SEQ ID NO: 1121 - RNA, 21 nt, Synthetic construct
  source 1..21: mol_type = other RNA
tatagaaatttaccaactgcc

SEQ ID NO: 1122 - RNA, 21 nt, Synthetic construct
  source 1..21: mol_type = other RNA
taatagaaatttaccaactgc

SEQ ID NO: 1123 - RNA, 21 nt, Synthetic construct
  source 1..21: mol_type = other RNA
tgaatagaaatttaccaactg

SEQ ID NO: 1124 - RNA, 21 nt, Synthetic construct
  source 1..21: mol_type = other RNA
tatggcaactgtttgaataga

SEQ ID NO: 1125 - RNA, 21 nt, Synthetic construct
  source 1..21: mol_type = other RNA
tgcatggcaactgtttgaata

SEQ ID NO: 1126 - RNA, 21 nt, Synthetic construct
  source 1..21: mol_type = other RNA
ttgcatggcaactgtttgaat

SEQ ID NO: 1127 - RNA, 21 nt, Synthetic construct
  source 1..21: mol_type = other RNA
tgtgcatggcaactgtttgaa

SEQ ID NO: 1128 - RNA, 21 nt, Synthetic construct
  source 1..21: mol_type = other RNA
tcaactggtgcatggcaactg

SEQ ID NO: 1129 - RNA, 21 nt, Synthetic construct
  source 1..21: mol_type = other RNA
tacaactggtgcatggcaact

SEQ ID NO: 1130 - RNA, 21 nt, Synthetic construct
  source 1..21: mol_type = other RNA
tgaacaactggtgcatggcaa

SEQ ID NO: 1131 - RNA, 21 nt, Synthetic construct
  source 1..21: mol_type = other RNA
ttgaacaactggtgcatggca

SEQ ID NO: 1132 - RNA, 21 nt, Synthetic construct
  source 1..21: mol_type = other RNA
tgtgaacaactggtgcatggc

SEQ ID NO: 1133 - RNA, 21 nt, Synthetic construct
  source 1..21: mol_type = other RNA
tgttgtgaacaactggtgcat

SEQ ID NO: 1134 - RNA, 21 nt, Synthetic construct
  source 1..21: mol_type = other RNA
tgtacccttgttgtgaacaac

SEQ ID NO: 1135 - RNA, 21 nt, Synthetic construct
  source 1..21: mol_type = other RNA
tttacgtacccttgttgtgaa

SEQ ID NO: 1136 - RNA, 21 nt, Synthetic construct
  source 1..21: mol_type = other RNA
tattacgtacccttgttgtga

SEQ ID NO: 1137 - RNA, 21 nt, Synthetic construct
  source 1..21: mol_type = other RNA
tgattacgtacccttgttgtg

SEQ ID NO: 1138 - RNA, 21 nt, Synthetic construct
  source 1..21: mol_type = other RNA
tagacagattacgtacccttg

SEQ ID NO: 1139 - RNA, 21 nt, Synthetic construct
  source 1..21: mol_type = other RNA
tccagacagattacgtaccct

SEQ ID NO: 1140 - RNA, 21 nt, Synthetic construct
  source 1..21: mol_type = other RNA
tatgccagacagattacgtac

SEQ ID NO: 1141 - RNA, 21 nt, Synthetic construct
  source 1..21: mol_type = other RNA
taatgccagacagattacgta

SEQ ID NO: 1142 - RNA, 21 nt, Synthetic construct
  source 1..21: mol_type = other RNA
tgtaatgccagacagattacg

SEQ ID NO: 1143 - RNA, 21 nt, Synthetic construct
  source 1..21: mol_type = other RNA
tagaagtaatgccagacagat

SEQ ID NO: 1144 - RNA, 21 nt, Synthetic construct
  source 1..21: mol_type = other RNA
tgtagaagtaatgccagacag

SEQ ID NO: 1145 - RNA, 21 nt, Synthetic construct
  source 1..21: mol_type = other RNA
taaagtagaagtaatgccaga

SEQ ID NO: 1146 - RNA, 21 nt, Synthetic construct
  source 1..21: mol_type = other RNA
taaaagtagaagtaatgccag

SEQ ID NO: 1147 - RNA, 21 nt, Synthetic construct
  source 1..21: mol_type = other RNA
tcaaaagtagaagtaatgcca

SEQ ID NO: 1148 - RNA, 21 nt, Synthetic construct
  source 1..21: mol_type = other RNA
tatcctttgtacaaaagtaga

SEQ ID NO: 1149 - RNA, 21 nt, Synthetic construct
  source 1..21: mol_type = other RNA
tgatcctttgtacaaaagtag

SEQ ID NO: 1150 - RNA, 21 nt, Synthetic construct
  source 1..21: mol_type = other RNA
ttgatcctttgtacaaaagta

SEQ ID NO: 1151 - RNA, 21 nt, Synthetic construct
  source 1..21: mol_type = other RNA
tttgatcctttgtacaaaagt

SEQ ID NO: 1152 - RNA, 21 nt, Synthetic construct
  source 1..21: mol_type = other RNA
ttttgatcctttgtacaaaag

(19) United States
(12) Patent Application Publication
(10) Pub. No.: US 2026/0098264 A1
(43) Pub. Date: Apr. 9, 2026

(54) COMPOSITIONS AND METHODS FOR TREATMENT OF NEUROLOGICAL DISORDERS

(71) Applicant: Atalanta Therapeutics, Inc., Boston, MA (US)

(72) Inventors: Corrie GALLANT-BEHM, Wellesley, MA (US); Wimalanathan KOKULAPALAN, Cambridge, MA (US); Matthew HASSLER, Boston, MA (US); Daniel CURTIS, Belmont, MA (US); Bruno Miguel DA CRUZ GODINHO, Reading, MA (US)

(21) Appl. No.: 19/115,144

(22) PCT Filed: Sep. 28, 2023
(86) PCT No.: PCT/US2023/075409

§ 371 Date: Mar. 25, 2025

Related U.S. Application Data
(60) Provisional application No. 63/410,810, filed on Sep. 28, 2022.

(51) Int. Cl. C12N 15/113

(52) U.S. Cl. CPC C12N 15/113; C12N 2310/14

(57) ABSTRACT

The present disclosure provides single- or double-stranded interfering RNA molecules (e.g., siRNA) that target a chromosome 9 open reading frame 72 (C9orf72) gene. The interfering RNA molecules may contain specific patterns of nucleoside modifications and internucleoside linkage modifications, as pharmaceutical compositions including the same. The siRNA molecules may be branched siRNA molecules, such as di-branched, tri-branched, ortetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having amyotrophic lateral sclerosis or frontotemporal dementia.

### SEQUENCE LISTING

The instant application contains a Sequence Listing which has been submitted electronically in XML format and is hereby incorporated by reference in its entirety. Said XML copy, created on Sep. 20, 2023, is named “51436-037WO2_Sequence_Listing_9_20_23” and is 1,011,545 bytes in size.

### TECHNICAL FIELD

This disclosure relates to small interfering RNA (siRNA) molecules, and compositions containing the same, that target RNA transcripts (e.g., mRNA) of a chromosome 9 open reading frame 72 (C9orf72) gene. The disclosure further describes methods for silencing of C9orf72 and the treatment of diseases that may benefit from the silencing of C9orf72 (e.g., frontotemporal dementia or amyotrophic lateral sclerosis) by delivering C9orf72-targeting siRNA molecules to a target tissue of a subject in need.

### BACKGROUND

C9orf72 (chromosome 9 open reading frame 72) encodes a protein that is implicated in the pathology of diseases including amyotrophic lateral sclerosis (ALS) and frontotemporal dementia (FTD). Studies have shown that repeat expansions within the C9orf72 can cause ALS and FTD. There is a need for therapeutics capable of selectively diminishing C9orf72 activity in a manner that provides effective treatment for ALS, FTD, or other C9orf72-related diseases or disorders.

### SUMMARY OF THE INVENTION

The present disclosure provides compositions and methods for reduction of chromosome 9 open reading frame 72 (C9orf72) expression by way of small interfering RNA (siRNA)-mediated silencing of C9orf72 transcripts. The compositions and methods provide the benefit of exhibiting high selectivity toward C9orf72 over other genes.

The siRNA molecules of the disclosure can be used to silence the C9orf72 gene, thereby preventing the translation of the corresponding mRNA transcript and reducing C9orf72 protein expression. This reduction of C9orf72 levels thus prevents disease onset or progression, as the hexanucleotide repeat expansion GGGGCC in the C9orf72 gene is a common cause of amyotrophic lateral sclerosis (ALS) and frontotemporal dementia (FTD). The siRNA molecules of the disclosure can be delivered directly to a subject in need of C9orf72 silencing by way of, for example, injection intrathecally, intracerebroventricularly, intrastriatally, intraparenchymally, intra-cisterna magna injection, such as by catheterization, intravenous injection, subcutaneous injection, or intramuscular injection.

In an aspect, the disclosure provides an siRNA molecule containing an antisense strand and sense strand having complementarity to the antisense strand. The antisense strand has complementarity sufficient to hybridize to a region within an C9orf72 mRNA transcript having the nucleic acid sequence of any one of SEQ ID NOS: 1-384. In some embodiments, the antisense strand has complementarity sufficient to hybridize to a region within an C9orf72 mRNA transcript having the nucleic acid sequence of any one of SEQ ID NOS: 1-192. In some embodiments, the antisense strand has complementarity sufficient to hybridize to a region within an C9orf72 mRNA transcript having the nucleic acid sequence of any one of SEQ ID NOs: 193-384. The antisense strand may be, for example, from 10 to 50 nucleotides in length (e.g., from 10 to 45 nucleotides in length, from 10 to 40 nucleotides in length, from 10 to 35 nucleotides in length, from 10 to 30 nucleotides in length, from 10 to 29 nucleotides in length, from 10 to 28 nucleotides in length, from 10 to 27 nucleotides in length, from 10 to 26 nucleotides in length, from 10 to 25 nucleotides in length, from 10 to 24 nucleotides in length, from 10 to 23 nucleotides in length, from 10 to 22 nucleotides in length, from 10 to 21 nucleotides in length, or from 10 to 20 nucleotides in length). In some embodiments, the antisense strand is 10 nucleotides in length, 11 nucleotides in length, 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, or more.

In some embodiments of any of the foregoing aspects, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 15 contiguous nucleobases within the C9orf72 mRNA transcript having the nucleic acid sequence of any one of SEQ ID NOs: 1-384, SEQ ID NOS: 1-192, or SEQ ID NOs: 193-384. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 16 contiguous nucleobases within the C9orf72 mRNA transcript having the nucleic acid sequence of any one of SEQ ID NOS: 1-384. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 17 contiguous nucleobases within the C9orf72 mRNA transcript having the nucleic acid sequence of any one of SEQ ID NOs: 1-384, SEQ ID NOS: 1-192, or SEQ ID NOS: 193-384. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 18 contiguous nucleobases within the C9orf72 mRNA transcript having the nucleic acid sequence of any one of SEQ ID NOs: 1-384, SEQ ID NOS: 1-192, or SEQ ID NOs: 193-384. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 19 contiguous nucleobases within the C9orf72 mRNA transcript having the nucleic acid sequence of any one of SEQ ID NOs: 1-384, SEQ ID NOS: 1-192, or SEQ ID NOS: 193-384. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 20 contiguous nucleobases within the C9orf72 mRNA transcript having the nucleic acid sequence of any one of SEQ ID NOS: 1-384, SEQ ID NOS: 1-192, or SEQ ID NOS: 193-384. In some embodiments, the antisense strand has at least 70% (e.g., at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to a region of 21 contiguous nucleobases within the C9orf72 mRNA transcript having the nucleic acid sequence of any one of SEQ ID NOS: 1-384, SEQ ID NOS: 1-192, or SEQ ID NOs: 193-384.

In some embodiments, the antisense strand has at least 70% (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) complementarity to the region within the C9orf72 mRNA transcript having the nucleic acid sequence of any one of SEQ ID NOS: 1-384. In some embodiments, the region within the C9orf72 mRNA transcript has the nucleic acid sequence of any one of SEQ ID NOs: 1-192. In some embodiments, the region within the C9orf72 mRNA transcript has the nucleic acid sequence of any one of SEQ ID NOs: 193-384.

In some embodiments, the antisense strand has at least 75% complementarity to the region within the C9orf72 mRNA transcript having the nucleic acid sequence of any one of SEQ ID NOs: 1-384. For example, the antisense strand may have at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the C9orf72 mRNA transcript having the nucleic acid sequence of any one of SEQ ID NOS: 1-384. In some embodiments, the region within the C9orf72 mRNA transcript has the nucleic acid sequence of any one of SEQ ID NOS: 1-192. In some embodiments, the region within the C9orf72 mRNA transcript has the nucleic acid sequence of any one of SEQ ID NOS: 193-384.

In some embodiments, the antisense strand has at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the C9orf72 mRNA transcript having the nucleic acid sequence of any one of SEQ ID NOS: 1-384. In some embodiments, the region of the C9orf72 mRNA transcript has the nucleic acid sequence of any one of SEQ ID NOs: 1-192. In some embodiments, the region of the C9orf72 mRNA transcript has the nucleic acid sequence of any one of SEQ ID NOs: 193-384.

In some embodiments, the antisense strand has from 10 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the C9orf72 mRNA transcript having the nucleic acid sequence of any one of SEQ ID NOs: 1-384. In some embodiments, the region of the C9orf72 mRNA transcript has the nucleic acid sequence of any one of SEQ ID NOS: 1-192. In some embodiments, the region of the C9orf72 mRNA transcript has the nucleic acid sequence of any one of SEQ ID NOs: 193-384.

In some embodiments, the antisense strand has from 12 to 30 contiguous nucleotides (e.g., 10, 11, 12, 13, 14, 15, 16, 17, 18, 19, 20, 21, 22, 23, 24, 25, 26, 27, 28, 29, or 30 contiguous nucleotides) that are fully complementary to a contiguous polynucleotide segment of equal length within the region of the C9orf72 mRNA transcript having the nucleic acid sequence of any one of SEQ ID NOS: 1-384. In some embodiments, the region of the C9orf72 mRNA transcript has the nucleic acid sequence of any one of SEQ ID NOs: 1-192. In some embodiments, the region of the C9orf72 mRNA transcript has the nucleic acid sequence of any one of SEQ ID NOs: 193-384.

In some embodiments, the antisense strand has from 15 to 30 contiguous nucleotides (e.g., 15, 16, 17, 18, 19, 20, 21, 22, 23, 24, 25, 26, 27, 28, 29, or 30 contiguous nucleotides) that are fully complementary to a contiguous polynucleotide segment of equal length within the region of the C9orf72 mRNA transcript having the nucleic acid sequence of any one of SEQ ID NOs: 1-384. In some embodiments, the region of the C9orf72 mRNA transcript has the nucleic acid sequence of any one of SEQ ID NOS: 1-192. In some embodiments, the region of the C9orf72 mRNA transcript has the nucleic acid sequence of any one of SEQ ID NOs: 193-384.

In some embodiments, the antisense strand has from 18 to 30 contiguous nucleotides (e.g., 18, 19, 20, 21, 22, 23, 24, 25, 26, 27, 28, 29, or 30 contiguous nucleotides) that are fully complementary to a contiguous polynucleotide segment of equal length within the region of the C9orf72 RNA transcript having the nucleic acid sequence of any one of SEQ ID NOs: 1-384. In some embodiments, the region of the C9orf72 mRNA transcript has the nucleic acid sequence of any one of SEQ ID NOS: 1-192. In some embodiments, the region of the C9orf72 mRNA transcript has the nucleic acid sequence of any one of SEQ ID NOs: 193-384.

In some embodiments, the antisense strand has from 18 to 25 contiguous nucleotides (e.g., 18, 19, 20, 21, 22, 23, 24, or 25 contiguous nucleotides) that are fully complementary to a contiguous polynucleotide segment of equal length within the region of the C9orf72 mRNA transcript having the nucleic acid sequence of any one of SEQ ID Nos: 1-384. In some embodiments, the region of the C9orf72 mRNA transcript has the nucleic acid sequence of any one of SEQ ID NOs: 1-192. In some embodiments, the region of the C9orf72 mRNA transcript has the nucleic acid sequence of any one of SEQ ID NOS: 193-384.

In some embodiments, the antisense strand has from 18 to 21 contiguous nucleotides (e.g., 18, 19, 20, or 21 contiguous nucleotides) that are fully complementary to a contiguous polynucleotide segment of equal length within the region of the C9orf72 mRNA transcript having the nucleic acid sequence of any one of SEQ ID NOs: 1-384. In some embodiments, the region of the C9orf72 mRNA transcript has the nucleic acid sequence of any one of SEQ ID NOS: 1-192. In some embodiments, the region of the C9orf72 mRNA transcript has the nucleic acid sequence of any one of SEQ ID NOS: 193-384.

In some embodiments, the antisense strand has 21 contiguous nucleotides that are fully complementary to a contiguous polynucleotide segment of equal length within the region of the C9orf72 mRNA transcript having the nucleic acid sequence of any one of SEQ ID NOs: 1-384. In some embodiments, the region of the C9orf72 mRNA transcript has the nucleic acid sequence of any one of SEQ ID NOS: 1-192. In some embodiments, the region of the C9orf72 mRNA transcript has the nucleic acid sequence of any one of SEQ ID NOs: 193-384.

In some embodiments, the antisense strand has 9 or fewer nucleotide mismatches relative to a region of 21 contiguous nucleobases of the C9orf72 RNA transcript having the nucleic acid sequence of any one of SEQ ID NOS: 1-384, optionally wherein the antisense strand contains 8 or fewer, 7 or fewer, 6 or fewer, 5 or fewer, 4 or fewer, 3 or fewer, 2 or fewer, or only 1 mismatch relative to the region of the C9orf72 mRNA transcript having the nucleic acid sequence of any one of SEQ ID NOs: 1-384. In some embodiments, the region of the C9orf72 mRNA transcript has the nucleic acid sequence of any one of SEQ ID NOS: 1-192. In some embodiments, the region of the C9orf72 mRNA transcript has the nucleic acid sequence of any one of SEQ ID NOs: 193-384.

In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 769-1152. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 769-960. In some embodiments, the antisense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 961-1152.

In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOS: 769-1152. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 769-960. In some embodiments, the antisense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 961-1152.

In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 769-1152, optionally wherein the antisense strand has a nucleic acid sequence that is at least 96%, 97%, 98%, or 99% identical to the nucleic acid sequence of any one of SEQ ID NOs: 769-1152. In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 769-960. In some embodiments, the antisense strand has a nucleic acid sequence that is at least 95% identical (e.g., 95%, 96%, 97%, 98%, 99%, or 100% identical) to the nucleic acid sequence of any one of SEQ ID NOs: 961-1152.

In some embodiments, the antisense strand has the nucleic acid sequence of any one of SEQ ID NOs: 769-1152. In some embodiments, the antisense strand has the nucleic acid sequence of any one of SEQ ID NOs: 769-960. In some embodiments the antisense strand has the nucleic acid sequence of any one of SEQ ID NOs: 961-1152.

In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 385-768. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOS: 385-576. In some embodiments, the sense strand has a nucleic acid sequence that is at least 85% identical (e.g., 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 577-768.

In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 385-768. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOS: 385-576. In some embodiments, the sense strand has a nucleic acid sequence that is at least 90% identical (e.g., 90%, 91%, 92%, 93%, 94%, 95%, 96%, 97%, 98%, 99%, or 100% identical) to the nucleic acid sequence of any one of SEQ ID NOs: 577-768.

In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 385-768, optionally wherein the sense strand has a nucleic acid sequence that is at least 96%, 97%, 98%, or 99% identical to the nucleic acid sequence of any one of SEQ ID NOS: 385-768. In some embodiments, the sense strand has a nucleic acid sequence that is at least 95% identical (e.g., 95%, 96%, 97%, 98%, 99%, or 100% identical) to the nucleic acid sequence of any one of SEQ ID NOS: 385-576. In some embodiments, the sense strand has a nucleic acid sequence that is at least 95% identical (e.g., 95%, 96%, 97%, 98%, 99%, or 100% identical) to the nucleic acid sequence of any one of SEQ ID NOs: 577-768.

In some embodiments, the siRNA molecule has a sense strand having the nucleic acid sequence of any one of SEQ ID NOS: 385-768. In some embodiments, the sense strand has a nucleic acid sequence of any one of SEQ ID NOS: 385-576. In some embodiments, the sense strand has a nucleic acid sequence of any one of SEQ ID NOs: 577-768.

In some embodiments, the antisense strand has a structure represented by Formula I, wherein Formula I is, in the 5′-to-3′ direction:

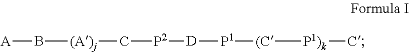

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A1, wherein Formula A1 is, in the 5′-to-3′ direction:

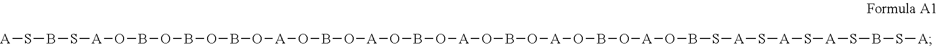

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula II, wherein Formula II is, in the 5′-to-3′ direction:

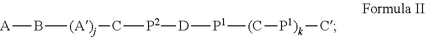

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, antisense strand has a structure represented by Formula A2, wherein Formula A2 is, in the 5′-to-3′ direction:

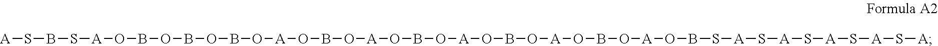

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula III, wherein Formula III is, in the 5′-to-3′ direction:

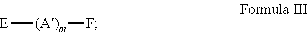

wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7 (e.g., 1, 2, 3, 4, 5, 6, or 7).

In some embodiments, the sense strand has a structure represented by Formula S1, wherein Formula S1 is, in the 5′-to-3′ direction:

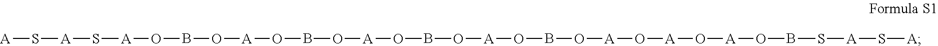

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S2, wherein Formula S2 is, in the 5′-to-3′ direction:

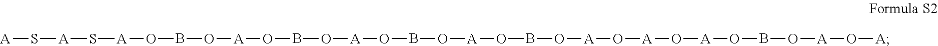

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S3, wherein Formula S3 is, in the 5′-to-3′ direction:

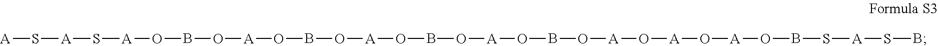

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S4, wherein Formula S4 is, in the 5′-to-3′ direction:

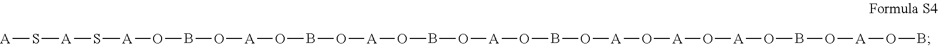

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula IV, wherein Formula IV is, in the 5′-to-3′ direction:

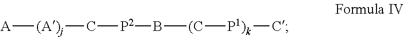

wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7 (e.g., 1, 2, 3, 4, 5, 6, or 7); and
k is an integer from 1 to 7 (e.g., 1, 2, 3, 4, 5, 6, or 7).

In some embodiments, the antisense strand has a structure represented by Formula A3, wherein Formula A3 is, in the 5′-to-3′ direction:

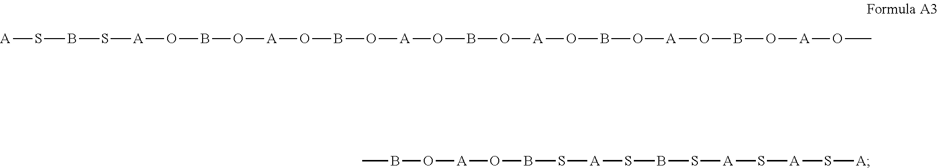

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula V, wherein Formula V is, in the 5′-to-3′ direction:

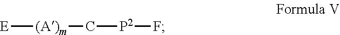

wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7 (e.g., 1, 2, 3, 4, 5, 6, or 7).

In some embodiments, the sense strand has a structure represented by Formula S5, wherein Formula S5 is, in the 5′-to-3′ direction:

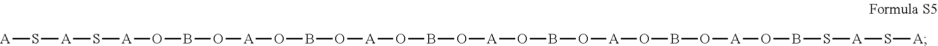

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S6, wherein Formula S6 is, in the 5′-to-3′ direction:

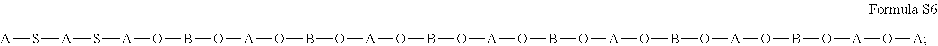

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S7, wherein Formula S7 is, in the 5′-to-3′ direction:

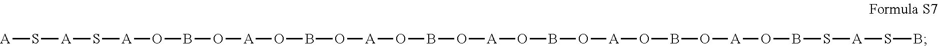

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula S8, wherein Formula S8 is, in the 5′-to-3′ direction:

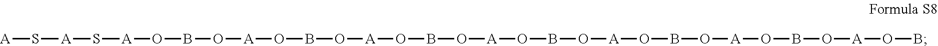

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand has a structure represented by Formula VI, wherein Formula VI is, in the 5′-to-3′ direction:

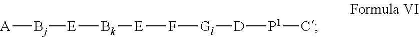

wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7 (e.g., 1, 2, 3, 4, 5, 6, or 7);
k is an integer from 1 to 7 (e.g., 1, 2, 3, 4, 5, 6, or 7); and
l is an integer from 1 to 7 (e.g., 1, 2, 3, 4, 5, 6, or 7).

In some embodiments, the antisense strand has a structure represented by Formula A4, wherein Formula A4 is, in the 5′-to-3′ direction:

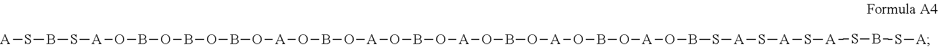

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand has a structure represented by Formula VII, wherein Formula VII is, in the 5′-to-3′ direction:

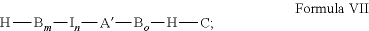

wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7 (e.g., 1, 2, 3, 4, 5, 6, or 7);
n is an integer from 1 to 7 (e.g., 1, 2, 3, 4, 5, 6, or 7); and
is an integer from 1 to 7 (e.g., 1, 2, 3, 4, 5, 6, or 7).
In some embodiments, the sense strand has a structure represented by Formula S9, wherein Formula S9 is, in the 5′-to-3′ direction:

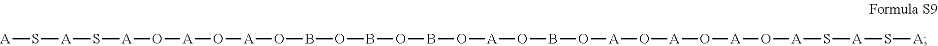

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand also has a 5′ phosphorus stabilizing moiety at the 5′ end of the antisense strand.

In some embodiments, the sense strand also has a 5′ phosphorus stabilizing moiety at the 5′ end of the sense strand.

In some embodiments, each 5′ phosphorus stabilizing moiety is, independently, represented by any one of Formulas IX, XX, XI, XII, XIII, XIV, XV, or XVI:

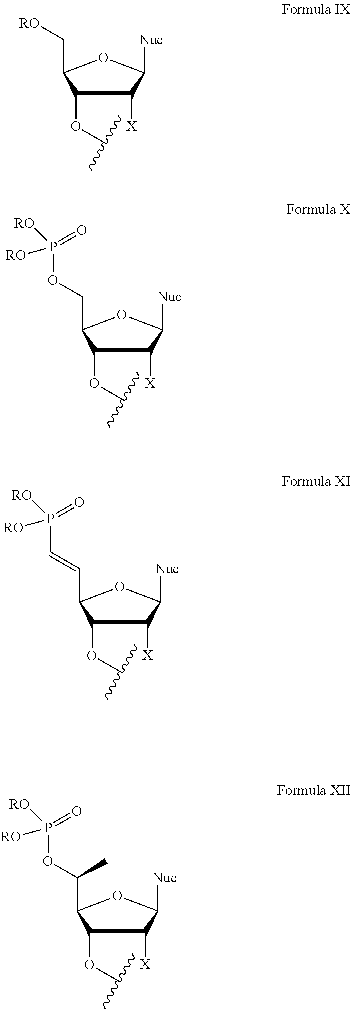

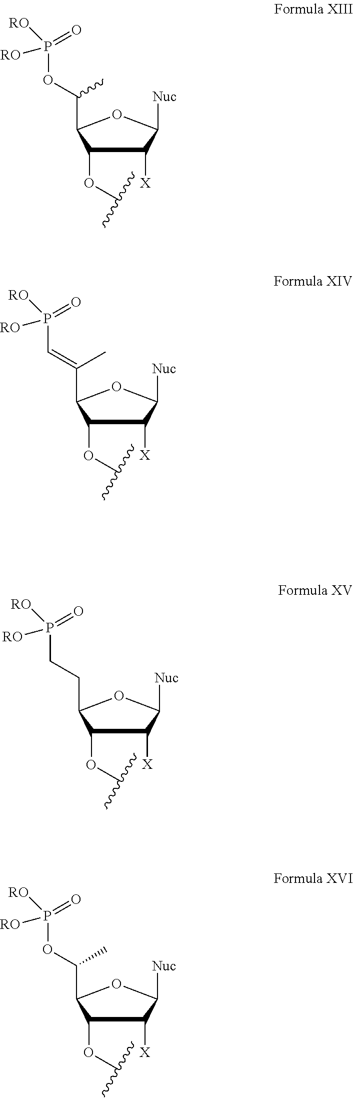

wherein Nuc represents a nucleobase, optionally wherein the nucleobase is selected from the group consisting of adenine, uracil, guanine, thymine, and cytosine, and R represents an optionally substituted alkyl, optionally substituted alkenyl, optionally substituted alkynyl, phenyl, benzyl, a cation (e.g., a monovalent cation), or hydrogen.

In some embodiments, the nucleobase is an adenine, uracil, guanine, thymine, or cytosine.

In some embodiments, the 5′ phosphorus stabilizing moiety is (E)-vinylphosphonate represented by Formula XI.

In some embodiments, the siRNA molecule also has a hydrophobic moiety at the 5′ or the 3′ end of the siRNA molecule.

In some embodiments, the hydrophobic moiety is selected from a group consisting of cholesterol, vitamin D, or tocopherol.

In some embodiments, the siRNA molecule is a branched siRNA molecule.

In some embodiments, the branched siRNA molecule is di-branched, tri-branched, or tetra-branched.

In some embodiments, the siRNA molecule is di-branched, optionally wherein the di-branched siRNA molecule is represented by any one of Formulas XVII, XVIII, or XIX:

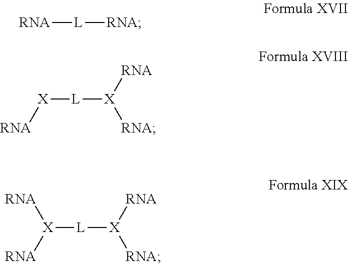

wherein each RNA is, independently, an siRNA molecule, L is a linker, and each X, independently, represents a branch point moiety.

In some embodiments, the di-branched siRNA molecule is represented by Formula XVII. In some embodiments, the di-branched siRNA molecule is represented by Formula XVIII. In some embodiments, the di-branched siRNA molecule is represented by Formula XIX.

In some embodiments, the siRNA molecule is tri-branched, optionally wherein the tri-branched siRNA molecule is represented by any one of Formulas XX, XXI, XXII, or XXIII:

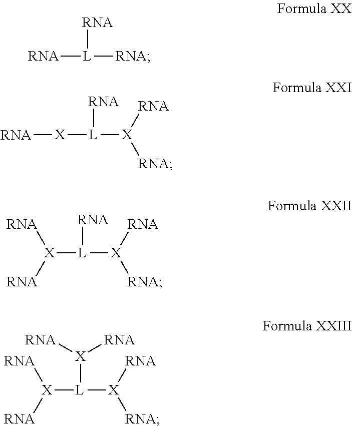

wherein each RNA is, independently, an siRNA molecule, L is a linker, and each X, independently, represents a branch point moiety.

In some embodiments, the tri-branched siRNA molecule is represented by Formula XX. In some embodiments, the tri-branched siRNA molecule is represented by Formula XXI. In some embodiments, the tri-branched siRNA molecule is represented by Formula XXII. In some embodiments, the tri-branched siRNA molecule is represented by Formula XXIII.

In some embodiments, the siRNA molecule is tetra-branched, optionally wherein the tetra-branched siRNA molecule is represented by any one of Formulas XXIV, XXV, XXVI, XXVII, or XXVIII:

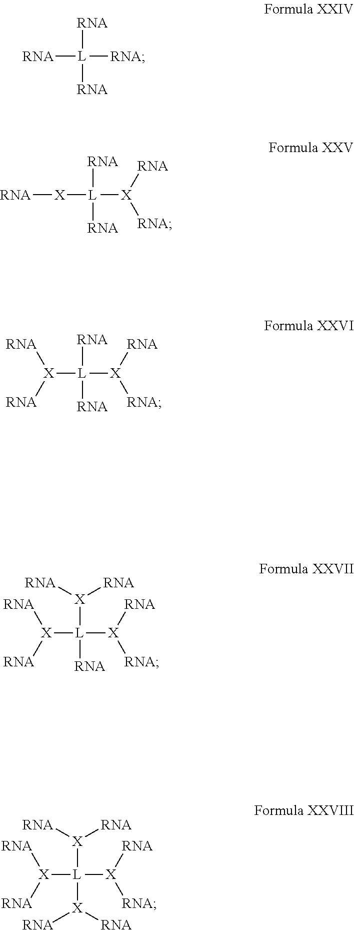

wherein each RNA is, independently, an siRNA molecule, L is a linker, and each X, independently, represents a branch point moiety.

In some embodiments, the tetra-branched siRNA molecule is represented by Formula XXIV. In some embodiments, the tetra-branched siRNA molecule is represented by Formula XXV. In some embodiments, the tetra-branched siRNA molecule is represented by Formula XXVI. In some embodiments, the tetra-branched siRNA molecule is represented by Formula XXVII. In some embodiments, the tetra-branched siRNA molecule is represented by Formula XXVIII.

In some embodiments of the branched siRNA, the linker is selected from a group consisting of one or more contiguous subunits of an ethylene glycol (e.g., polyethylene glycol (PEG), such as, e.g., triethylene glycol (TrEG) or tetraethylene glycol (TEG)), alkyl, carbohydrate, block copolymer, peptide, RNA, and DNA.

In some embodiments, the linker is an ethylene glycol oligomer. In some embodiments, the linker is an alkyl oligomer. In some embodiments, the linker is a carbohydrate oligomer. In some embodiments, the linker is a block copolymer. In some embodiments, the linker is a peptide oligomer. In some embodiments, the linker is an RNA oligomer. In some embodiments, the linker is a DNA oligomer.

In some embodiments, the ethylene glycol oligomer is a PEG. In some embodiments, the PEG is a TrEG. In some embodiments, the PEG is a TEG.

In some embodiments, the oligomer or copolymer contains 2 to 20 contiguous subunits (e.g., 2, 3, 4, 5, 6, 7, 8, 9, 10, 11, 12, 13, 14, 15, 16, 17, 18, 19, or 20 contiguous subunits).

In some embodiments, the linker attaches one or more (e.g., 1, 2, 3, 4, or more) siRNA molecules by way of a covalent bond-forming moiety.

In some embodiments, the covalent bond-forming moiety is selected from the group consisting of an alkyl, ester, amide, carbamate, phosphonate, phosphate, phosphorothioate, phosphoroamidate, triazole, urea, and formacetal.

In some embodiments, the linker includes a structure of Formula L1:

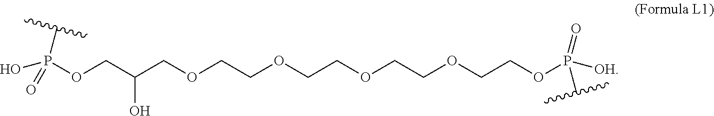 [SMILES: *P(=O)(O)OCCOCCOCCOCCOCC(O)COP(*)(=O)O]

In some embodiments, the linker includes a structure of Formula L2:

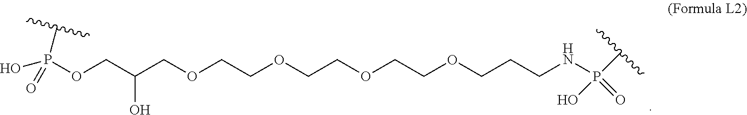 [SMILES: *P(=O)(O)NCCCOCCOCCOCCOCC(O)COP(*)(=O)O]

In some embodiments, the linker includes a structure of Formula L3:

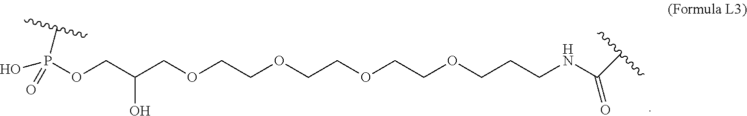 [SMILES: *C(=O)NCCCOCCOCCOCCOCC(O)COP(*)(=O)O]

In some embodiments, the linker includes a structure of Formula L4:

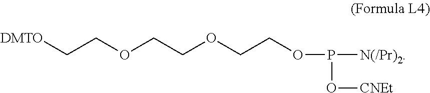

In some embodiments, the linker includes a structure of Formula L5:

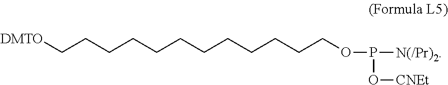

In some embodiments, the linker includes a structure of Formula L6:

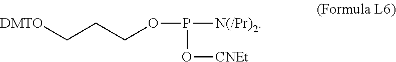

In some embodiments, the linker includes a structure of Formula L7:

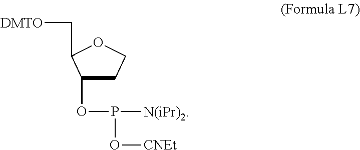

In some embodiments, the linker includes a structure of Formula L8:

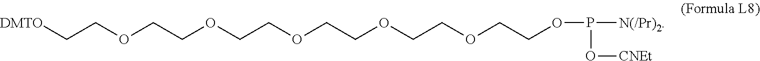

In some embodiments, the linker includes a structure of Formula L9:

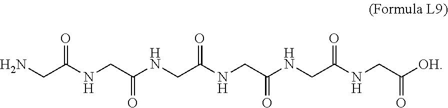 [SMILES: NCC(=O)NCC(=O)NCC(=O)NCC(=O)NCC(=O)NCC(=O)O]

In some embodiments of any of the siRNA molecules described herein, 50% or more of the ribonucleotides in the antisense strand are 2′-O-Me ribonucleotides (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 60% or more of the ribonucleotides in the antisense strand are 2′-O-Me ribonucleotides (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 70% or more of the ribonucleotides in the antisense strand are 2′-O-Me ribonucleotides (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 80% or more of the ribonucleotides in the antisense strand are 2′-O-Me ribonucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% of the ribonucleotides in the antisense strand may be 2′-O-Me ribonucleotides).

In some embodiments, 90% or more of the ribonucleotides in the antisense strand are 2′-O-Me ribonucleotides (e.g., 90%, 91%, 92%, 93%, 94%, 95%, 96%, 97%, 98%, 99%, or 100% of the ribonucleotides in the antisense strand may be 2′-O-Me ribonucleotides).

In some embodiments, 10% or less of the internucleoside linkages are phosphodiester linkages or phosphorothioate linkages. In some embodiments, at least 10%, 20%, 30%, 40%, 50%, 60%, 70%, 80%, or 90% of the internucleoside linkages are phosphodiester linkages or phosphorothioate linkages. In some embodiments, 100% of the internucleoside linkages are phosphodiester linkages or phosphorothioate linkages.

In some embodiments, 9 internucleoside linkages are phosphodiester linkages or phosphorothioate linkages.

In some embodiments, the length of the antisense strand is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the length of the antisense strand is 20 nucleotides. In some embodiments, the length of the antisense strand is 21 nucleotides. In some embodiments, the length of the antisense strand is 22 nucleotides. In some embodiments, the length of the antisense strand is 23 nucleotides.

In some embodiments, the length of the antisense strand is 24 nucleotides. In some embodiments, the length of the antisense strand is 25 nucleotides. In some embodiments, the length of the antisense strand is 26 nucleotides. In some embodiments, the length of the antisense strand is 27 nucleotides. In some embodiments, the length of the antisense strand is 28 nucleotides. In some embodiments, the length of the antisense strand is 29 nucleotides. In some embodiments, the length of the antisense strand is 30 nucleotides.

In some embodiments, the siRNA molecules of the branched compound are joined to one another by way of a linker (e.g., an ethylene glycol oligomer, such as tetraethylene glycol). In some embodiments, the siRNA molecules of the branched compound are joined to one another by way of a linker between the sense strand of one siRNA molecule and the sense strand of the other siRNA molecule. In some embodiments, the siRNA molecules are joined by way of linkers between the antisense strand of one siRNA molecule and the antisense strand of the other siRNA molecule. In some embodiments, the siRNA molecules of the branched compound are joined to one another by way of a linker between the sense strand of one siRNA molecule and the antisense strand of the other siRNA molecule.

In some embodiments, the length of the sense strand is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 18 nucleotides (e.g., 14 nucleotides, 15 nucleotides, 16 nucleotides, 17 nucleotides, or 18 nucleotides). In some embodiments, the length of the sense strand is 15 nucleotides. In some embodiments, the length of the sense strand is 16 nucleotides. In some embodiments, the length of the sense strand is 17 nucleotides. In some embodiments, the length of the sense strand is 18 nucleotides. In some embodiments, the length of the sense strand is 19 nucleotides. In some embodiments, the length of the sense strand is 20 nucleotides. In some embodiments, the length of the sense strand is 21 nucleotides. In some embodiments, the length of the sense strand is 22 nucleotides. In some embodiments, the length of the sense strand is 23 nucleotides. In some embodiments, the length of the sense strand is 24 nucleotides. In some embodiments, the length of the sense strand is 25 nucleotides. In some embodiments, the length of the sense strand is 26 nucleotides. In some embodiments, the length of the sense strand is 27 nucleotides. In some embodiments, the length of the sense strand is 28 nucleotides. In some embodiments, the length of the sense strand is 29 nucleotides. In some embodiments, the length of the sense strand is 30 nucleotides.

In some embodiments, four internucleoside linkages are phosphorothioate linkages.

In some embodiments of the siRNA molecules described herein, the antisense strand is 18 nucleotides in length and the sense strand is 14 nucleotides in length. In some embodiments, the antisense strand is 18 nucleotides in length and the sense strand is 15 nucleotides in length. In some embodiments, the antisense strand is 18 nucleotides in length and the sense strand is 16 nucleotides in length. In some embodiments, the antisense strand is 18 nucleotides in length and the sense strand is 17 nucleotides in length. In some embodiments, the antisense strand is 18 nucleotides in length and the sense strand is 18 nucleotides in length. In some embodiments, the antisense strand is 19 nucleotides in length and the sense strand is 14 nucleotides in length. In some embodiments, the antisense strand is 19 nucleotides in length and the sense strand is 15 nucleotides in length. In some embodiments, the antisense strand is 19 nucleotides in length and the sense strand is 16 nucleotides in length. In some embodiments, the antisense strand is 19 nucleotides in length and the sense strand is 17 nucleotides in length. In some embodiments, the antisense strand is 19 nucleotides in length and the sense strand is 18 nucleotides in length. In some embodiments, the antisense strand is 19 nucleotides in length and the sense strand is 19 nucleotides in length. In some embodiments, the antisense strand is 20 nucleotides in length and the sense strand is 14 nucleotides in length. In some embodiments, the antisense strand is 20 nucleotides in length and the sense strand is 15 nucleotides in length. In some embodiments, the antisense strand is 20 nucleotides in length and the sense strand is 16 nucleotides in length. In some embodiments, the antisense strand is 20 nucleotides in length and the sense strand is 17 nucleotides in length. In some embodiments, the antisense strand is 20 nucleotides in length and the sense strand is 18 nucleotides in length. In some embodiments, the antisense strand is 20 nucleotides in length and the sense strand is 19 nucleotides in length. In some embodiments, the antisense strand is 20 nucleotides in length and the sense strand is 20 nucleotides in length. In some embodiments, the antisense strand is 21 nucleotides in length and the sense strand is 14 nucleotides in length. In some embodiments, the antisense strand is 21 nucleotides in length and the sense strand is 15 nucleotides in length. In some embodiments, the antisense strand is 21 nucleotides in length and the sense strand is 16 nucleotides in length. In some embodiments, the antisense strand is 21 nucleotides in length and the sense strand is 17 nucleotides in length. In some embodiments, the antisense strand is 21 nucleotides in length and the sense strand is 18 nucleotides in length. In some embodiments, the antisense strand is 21 nucleotides in length and the sense strand is 19 nucleotides in length. In some embodiments, the antisense strand is 21 nucleotides in length and the sense strand is 20 nucleotides in length. In some embodiments, the antisense strand is 21 nucleotides in length and the sense strand is 21 nucleotides in length. In some embodiments, the antisense strand is 22 nucleotides in length and the sense strand is 14 nucleotides in length. In some embodiments, the antisense strand is 22 nucleotides in length and the sense strand is 15 nucleotides in length. In some embodiments, the antisense strand is 22 nucleotides in length and the sense strand is 16 nucleotides in length. In some embodiments, the antisense strand is 22 nucleotides in length and the sense strand is 17 nucleotides in length. In some embodiments, the antisense strand is 22 nucleotides in length and the sense strand is 18 nucleotides in length. In some embodiments, the antisense strand is 22 nucleotides in length and the sense strand is 19 nucleotides in length. In some embodiments, the antisense strand is 22 nucleotides in length and the sense strand is 20 nucleotides in length. In some embodiments, the antisense strand is 22 nucleotides in length and the sense strand is 21 nucleotides in length. In some embodiments, the antisense strand is 22 nucleotides in length and the sense strand is 22 nucleotides in length. In some embodiments, the antisense strand is 23 nucleotides in length and the sense strand is 14 nucleotides in length. In some embodiments, the antisense strand is 23 nucleotides in length and the sense strand is 15 nucleotides in length. In some embodiments, the antisense strand is 23 nucleotides in length and the sense strand is 16 nucleotides in length. In some embodiments, the antisense strand is 23 nucleotides in length and the sense strand is 17 nucleotides in length. In some embodiments, the antisense strand is 23 nucleotides in length and the sense strand is 18 nucleotides in length. In some embodiments, the antisense strand is 23 nucleotides in length and the sense strand is 19 nucleotides in length. In some embodiments, the antisense strand is 23 nucleotides in length and the sense strand is 20 nucleotides in length. In some embodiments, the antisense strand is 23 nucleotides in length and the sense strand is 21 nucleotides in length. In some embodiments, the antisense strand is 23 nucleotides in length and the sense strand is 22 nucleotides in length. In some embodiments, the antisense strand is 23 nucleotides in length and the sense strand is 23 nucleotides in length. In some embodiments, the antisense strand is 24 nucleotides in length and the sense strand is 14 nucleotides in length. In some embodiments, the antisense strand is 24 nucleotides in length and the sense strand is 15 nucleotides in length. In some embodiments, the antisense strand is 24 nucleotides in length and the sense strand is 16 nucleotides in length. In some embodiments, the antisense strand is 24 nucleotides in length and the sense strand is 17 nucleotides in length. In some embodiments, the antisense strand is 24 nucleotides in length and the sense strand is 18 nucleotides in length. In some embodiments, the antisense strand is 24 nucleotides in length and the sense strand is 19 nucleotides in length. In some embodiments, the antisense strand is 24 nucleotides in length and the sense strand is 20 nucleotides in length. In some embodiments, the antisense strand is 24 nucleotides in length and the sense strand is 21 nucleotides in length. In some embodiments, the antisense strand is 24 nucleotides in length and the sense strand is 22 nucleotides in length. In some embodiments, the antisense strand is 24 nucleotides in length and the sense strand is 23 nucleotides in length. In some embodiments, the antisense strand is 24 nucleotides in length and the sense strand is 24 nucleotides in length. In some embodiments, the antisense strand is 25 nucleotides in length and the sense strand is 14 nucleotides in length. In some embodiments, the antisense strand is 25 nucleotides in length and the sense strand is 15 nucleotides in length. In some embodiments, the antisense strand is 25 nucleotides in length and the sense strand is 16 nucleotides in length. In some embodiments, the antisense strand is 25 nucleotides in length and the sense strand is 17 nucleotides in length. In some embodiments, the antisense strand is 25 nucleotides in length and the sense strand is 18 nucleotides in length. In some embodiments, the antisense strand is 25 nucleotides in length and the sense strand is 19 nucleotides in length. In some embodiments, the antisense strand is 25 nucleotides in length and the sense strand is 20 nucleotides in length. In some embodiments, the antisense strand is 25 nucleotides in length and the sense strand is 21 nucleotides in length. In some embodiments, the antisense strand is 25 nucleotides in length and the sense strand is 22 nucleotides in length. In some embodiments, the antisense strand is 25 nucleotides in length and the sense strand is 23 nucleotides in length. In some embodiments, the antisense strand is 25 nucleotides in length and the sense strand is 24 nucleotides in length. In some embodiments, the antisense strand is 25 nucleotides in length and the sense strand is 25 nucleotides in length. In some embodiments, the antisense strand is 26 nucleotides in length and the sense strand is 14 nucleotides in length. In some embodiments, the antisense strand is 26 nucleotides in length and the sense strand is 15 nucleotides in length. In some embodiments, the antisense strand is 26 nucleotides in length and the sense strand is 16 nucleotides in length. In some embodiments, the antisense strand is 26 nucleotides in length and the sense strand is 17 nucleotides in length. In some embodiments, the antisense strand is 26 nucleotides in length and the sense strand is 18 nucleotides in length. In some embodiments, the antisense strand is 26 nucleotides in length and the sense strand is 19 nucleotides in length. In some embodiments, the antisense strand is 26 nucleotides in length and the sense strand is 20 nucleotides in length. In some embodiments, the antisense strand is 26 nucleotides in length and the sense strand is 21 nucleotides in length. In some embodiments, the antisense strand is 26 nucleotides in length and the sense strand is 22 nucleotides in length. In some embodiments, the antisense strand is 26 nucleotides in length and the sense strand is 23 nucleotides in length. In some embodiments, the antisense strand is 26 nucleotides in length and the sense strand is 24 nucleotides in length. In some embodiments, the antisense strand is 26 nucleotides in length and the sense strand is 25 nucleotides in length. In some embodiments, the antisense strand is 26 nucleotides in length and the sense strand is 26 nucleotides in length. In some embodiments, the antisense strand is 27 nucleotides in length and the sense strand is 14 nucleotides in length. In some embodiments, the antisense strand is 27 nucleotides in length and the sense strand is 15 nucleotides in length. In some embodiments, the antisense strand is 27 nucleotides in length and the sense strand is 16 nucleotides in length. In some embodiments, the antisense strand is 27 nucleotides in length and the sense strand is 17 nucleotides in length. In some embodiments, the antisense strand is 27 nucleotides in length and the sense strand is 18 nucleotides in length. In some embodiments, the antisense strand is 27 nucleotides in length and the sense strand is 19 nucleotides in length. In some embodiments, the antisense strand is 27 nucleotides in length and the sense strand is 20 nucleotides in length. In some embodiments, the antisense strand is 27 nucleotides in length and the sense strand is 21 nucleotides in length. In some embodiments, the antisense strand is 27 nucleotides in length and the sense strand is 22 nucleotides in length. In some embodiments, the antisense strand is 27 nucleotides in length and the sense strand is 23 nucleotides in length. In some embodiments, the antisense strand is 27 nucleotides in length and the sense strand is 24 nucleotides in length. In some embodiments, the antisense strand is 27 nucleotides in length and the sense strand is 25 nucleotides in length. In some embodiments, the antisense strand is 27 nucleotides in length and the sense strand is 26 nucleotides in length. In some embodiments, the antisense strand is 27 nucleotides in length and the sense strand is 27 nucleotides in length. In some embodiments, the antisense strand is 28 nucleotides in length and the sense strand is 14 nucleotides in length. In some embodiments, the antisense strand is 28 nucleotides in length and the sense strand is 15 nucleotides in length. In some embodiments, the antisense strand is 28 nucleotides in length and the sense strand is 16 nucleotides in length. In some embodiments, the antisense strand is 28 nucleotides in length and the sense strand is 17 nucleotides in length. In some embodiments, the antisense strand is 28 nucleotides in length and the sense strand is 18 nucleotides in length. In some embodiments, the antisense strand is 28 nucleotides in length and the sense strand is 19 nucleotides in length. In some embodiments, the antisense strand is 28 nucleotides in length and the sense strand is 20 nucleotides in length. In some embodiments, the antisense strand is 28 nucleotides in length and the sense strand is 21 nucleotides in length. In some embodiments, the antisense strand is 28 nucleotides in length and the sense strand is 22 nucleotides in length. In some embodiments, the antisense strand is 28 nucleotides in length and the sense strand is 23 nucleotides in length. In some embodiments, the antisense strand is 28 nucleotides in length and the sense strand is 24 nucleotides in length. In some embodiments, the antisense strand is 28 nucleotides in length and the sense strand is 25 nucleotides in length. In some embodiments, the antisense strand is 28 nucleotides in length and the sense strand is 26 nucleotides in length. In some embodiments, the antisense strand is 28 nucleotides in length and the sense strand is 27 nucleotides in length. In some embodiments, the antisense strand is 28 nucleotides in length and the sense strand is 28 nucleotides in length. In some embodiments, the antisense strand is 29 nucleotides in length and the sense strand is 14 nucleotides in length. In some embodiments, the antisense strand is 29 nucleotides in length and the sense strand is 15 nucleotides in length. In some embodiments, the antisense strand is 29 nucleotides in length and the sense strand is 16 nucleotides in length. In some embodiments, the antisense strand is 29 nucleotides in length and the sense strand is 17 nucleotides in length. In some embodiments, the antisense strand is 29 nucleotides in length and the sense strand is 18 nucleotides in length. In some embodiments, the antisense strand is 29 nucleotides in length and the sense strand is 19 nucleotides in length. In some embodiments, the antisense strand is 29 nucleotides in length and the sense strand is 20 nucleotides in length. In some embodiments, the antisense strand is 29 nucleotides in length and the sense strand is 21 nucleotides in length. In some embodiments, the antisense strand is 29 nucleotides in length and the sense strand is 22 nucleotides in length. In some embodiments, the antisense strand is 29 nucleotides in length and the sense strand is 23 nucleotides in length. In some embodiments, the antisense strand is 29 nucleotides in length and the sense strand is 24 nucleotides in length. In some embodiments, the antisense strand is 29 nucleotides in length and the sense strand is 25 nucleotides in length. In some embodiments, the antisense strand is 29 nucleotides in length and the sense strand is 26 nucleotides in length. In some embodiments, the antisense strand is 29 nucleotides in length and the sense strand is 27 nucleotides in length. In some embodiments, the antisense strand is 29 nucleotides in length and the sense strand is 28 nucleotides in length. In some embodiments, the antisense strand is 29 nucleotides in length and the sense strand is 29 nucleotides in length. In some embodiments, the antisense strand is 30 nucleotides in length and the sense strand is 14 nucleotides in length. In some embodiments, the antisense strand is 30 nucleotides in length and the sense strand is 15 nucleotides in length. In some embodiments, the antisense strand is 30 nucleotides in length and the sense strand is 16 nucleotides in length. In some embodiments, the antisense strand is 30 nucleotides in length and the sense strand is 17 nucleotides in length. In some embodiments, the antisense strand is 30 nucleotides in length and the sense strand is 18 nucleotides in length. In some embodiments, the antisense strand is 30 nucleotides in length and the sense strand is 19 nucleotides in length. In some embodiments, the antisense strand is 30 nucleotides in length and the sense strand is 20 nucleotides in length. In some embodiments, the antisense strand is 30 nucleotides in length and the sense strand is 21 nucleotides in length. In some embodiments, the antisense strand is 30 nucleotides in length and the sense strand is 22 nucleotides in length. In some embodiments, the antisense strand is 30 nucleotides in length and the sense strand is 23 nucleotides in length. In some embodiments, the antisense strand is 30 nucleotides in length and the sense strand is 24 nucleotides in length. In some embodiments, the antisense strand is 30 nucleotides in length and the sense strand is 25 nucleotides in length. In some embodiments, the antisense strand is 30 nucleotides in length and the sense strand is 26 nucleotides in length. In some embodiments, the antisense strand is 30 nucleotides in length and the sense strand is 27 nucleotides in length. In some embodiments, the antisense strand is 30 nucleotides in length and the sense strand is 28 nucleotides in length. In some embodiments, the antisense strand is 30 nucleotides in length and the sense strand is 29 nucleotides in length. In some embodiments, the antisense strand is 30 nucleotides in length and the sense strand is 30 nucleotides in length.

In a further aspect, the disclosure provides a pharmaceutical composition containing an siRNA molecule of any of the preceding aspects or embodiments of the disclosure, and a pharmaceutically acceptable excipient, carrier, or diluent.

In a further aspect, the disclosure provides a method of delivering an siRNA molecule to a subject diagnosed as having ALS by administering a therapeutically effective amount of the siRNA molecule or a pharmaceutical composition of any of the preceding aspects or embodiments of the disclosure to the subject.

In a further aspect, the disclosure provides a method of delivering an siRNA molecule to a subject diagnosed as having FTD by administering a therapeutically effective amount of the siRNA molecule or a pharmaceutical composition of any of the preceding aspects or embodiments of the disclosure to the subject.

In a further aspect, the disclosure provides a method of treating ALS in a subject in need thereof by administering a therapeutically effective amount of an siRNA molecule or a pharmaceutical composition of any of the preceding aspects or embodiments of the disclosure to the subject.

In a further aspect, the disclosure provides a method of treating FTD in a subject in need thereof by administering a therapeutically effective amount of an siRNA molecule or a pharmaceutical composition of any of the preceding aspects or embodiments of the disclosure to the subject.

In another aspect, the disclosure provides a method of reducing C9orf72 expression in a subject in need thereof by administering a therapeutically effective amount of an siRNA or pharmaceutical composition of any of the preceding aspects or embodiments of the disclosure.

In some embodiments, the siRNA molecule or the pharmaceutical composition is administered to the subject by way of intracerebroventricular, intrastriatal, intraparenchymal or intrathecal injection. In some embodiments, the siRNA molecule or the pharmaceutical composition is administered to the subject by way of intravenous, intramuscular, or subcutaneous injection.

In some embodiments, the subject is a human.

In another aspect, the disclosure provides a kit containing an siRNA molecule or pharmaceutical composition of any of the preceding aspects or embodiments of the disclosure, and a package insert that instructs a user of the kit to perform the method of any of the preceding aspects or embodiments of the disclosure.

### Definitions

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively.

As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group, or a variant thereof, on its 3′ or 5′ sugar hydroxyl group. Examples of phosphate group variants include, but are not limited to, saturated alkyl phosphonates, unsaturated alkenyl phosphonates, phosphorothioates, and phosphoramidites.

In the context of this disclosure, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring (e.g., modified) portions that function similarly. Such modified or substituted oligonucleotides are often preferred over native forms because of desirable properties such as, for example, enhanced cellular uptake, enhanced affinity for nucleic acid target and increased stability in the presence of nucleases.

As used herein, the term “siRNA” refers to small interfering RNA duplexes that induce the RNA interference (RNAi) pathway. siRNA molecules may vary in length (generally, between 10 and 30 base pairs) and may contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

The term “interfering RNA molecule” refers to an RNA molecule, such as a small interfering RNA (siRNA), microRNA (miRNA), short hairpin RNA (shRNA), or an antisense oligonucleotide (ASO) that suppresses the endogenous function of a target RNA transcript.

As used herein, the terms “express” and “expression” refer to one or more of the following events: (1) production of an RNA template from a DNA sequence (e.g., by transcription); (2) processing of an RNA transcript (e.g., by splicing, editing, 5′ cap formation, and/or 3′ end processing); and (3) translation of an RNA into a polypeptide or protein. In the context of a gene that encodes a protein product, the terms “gene expression” and the like are used interchangeably with the terms “protein expression” and the like. Expression of a gene or protein of interest in a patient can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the patient. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the terms “target,” “targeting,” and “targeted,” in the context of the design of an siRNA, refers to generating an antisense strand so as to anneal the antisense strand to a region within the mRNA transcript of interest in a manner that results in a reduction in translation of the mRNA into the protein product.

As used herein, the terms “chemically modified nucleotide,” “nucleotide analog,” “altered nucleotide,” and “modified nucleotide” refer to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function.

As used herein, the term “metabolically stabilized” refers to RNA molecules that contain ribonucleotides that have been chemically modified in order to decrease the rate of metabolism of an RNA molecule that is administered to a subject. Exemplary modifications include 2′-hydroxy to 2′-O-methoxy or 2′-fluoro, and phosphodiester to phosphorothioate.

As used herein, the term “phosphorothioate” refers to a phosphate group of a nucleotide that is modified by substituting one or more of the oxygens of the phosphate group with sulfur.

As used herein, the terms “internucleoside” and “internucleotide” refer to the bonds between nucleosides and nucleotides, respectively.

As used herein, the term “antagomirs” refers to nucleic acids that can function as inhibitors of miRNA activity.

As used herein, the term “gapmers” refers to chimeric antisense nucleic acids that contain a central block of deoxynucleotide monomers sufficiently long to induce RNase H cleavage. The deoxynucleotide block is flanked by ribonucleotide monomers or ribonucleotide monomers containing modifications.

As used herein, the term “mixmers” refers to nucleic acids that contain a mix of locked nucleic acids (LNAs) and DNA.

As used herein, the term “guide RNAs” refers to nucleic acids that have sequence complementarity to a specific sequence in the genome immediately or 1 base pair upstream of the protospacer adjacent motif (PAM) sequence as used in CRISPR/Cas9 gene editing systems. Alternatively, “guide RNAs” may refer to nucleic acids that have sequence complementarity (e.g., are antisense) to a specific messenger RNA (mRNA) sequence. In this context, a guide RNA may also have sequence complementarity to a “passenger RNA” sequence of equal or shorter length, which is identical or substantially identical to the sequence of mRNA to which the guide RNA hybridizes.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 SiRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

The term “phosphate moiety” as used herein, refers to a terminal phosphate group that includes phosphates as well as modified phosphates. The phosphate moiety may be located at either terminus but is preferred at the 5′-terminal nucleoside. In one aspect, the terminal phosphate is unmodified having the formula —O—P(—O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group may include from 1 to 3 phosphate moieties that are each, independently, unmodified (di- or tri-phosphates) or modified.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety may be located at either terminus but is preferred at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group may include from 1 to 3 phosphate moieties that are each, independently, unmodified (di- or tri-phosphates) or modified.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143. Certain of the above-referenced modifications (e.g., phosphate group modifications) preferably decrease the rate of hydrolysis of, for example, polynucleotides including said analogs in vivo or in vitro.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

$$100 multiplied by ( the fraction X / Y )$$

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

$$100 multiplied by ( the fraction X / Y )$$

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The term “complementarity sufficient to hybridize,” as used herein, refers to a nucleic acid sequence or a portion thereof that need not be fully complementary (e.g., 100% complementary) to a target region or a nucleic acid sequence or a portion thereof that has one or more nucleotide mismatches relative to the target region but that is still capable of hybridizing to the target region under specified conditions. For example, the nucleic acid may be, e.g., 95% complementary, 90%, complementary, 85% complementary, 80% complementary, 75% complementary, 70% complementary, 65% complementary, 60% complementary, 55% complementary, 50% complementary, or less, but still form sufficient base pairs with the target so as to hybridize across its length.

“Hybridization” or “annealing” of nucleic acids is achieved when one or more nucleoside residues within a polynucleotide base pairs with one or more complementary nucleosides to form a stable duplex. The base pairing is typically driven by hydrogen bonding events. Hybridization includes Watson-Crick base pairs formed from natural and/or modified nucleobases. The hybridization can also include non-Watson-Crick base pairs, such as wobble base pairs (guanosine-uracil, hypoxanthine-uracil, hypoxanthine-adenine, and hypoxanthine-cytosine) and Hoogsteen base pairs. Nucleic acids need not be 100% complementary to undergo hybridization. For example, one nucleic acid may be, e.g., 95% complementary, 90%, complementary, 85% complementary, 80% complementary, 75% complementary, 70% complementary, 65% complementary, 60% complementary, 55% complementary, 50% complementary, or less, relative to another nucleic acid, but the two nucleic acids may still form sufficient base pairs with one another so as to hybridize.

The “stable duplex” formed upon the annealing/hybridization of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

The term “gene silencing” refers to the suppression of gene expression, e.g., endogenous gene expression of C9orf72, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner by way of RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C≡C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C≡CH and —C≡C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group may be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl group generally has the formula of phenyl —CH₂—. A benzyl group may be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “triazole” refers to heterocyclic compounds with the formula (C₂H₃N₃), having a five-membered ring of two carbons and three nitrogens, the positions of which can change resulting in multiple isomers.

As used herein, the term “terminal group” refers to the group at which a carbon chain or nucleic acid ends.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

As used herein, the term “lipophilic amino acid” refers to an amino acid including a hydrophobic moiety (e.g., an alkyl chain or an aromatic ring).

As used herein, the term “target of delivery” refers to the organ or part of the body to which it is desired to deliver the branched oligonucleotide compositions.

As used herein, the term “between X and Y” is inclusive of the values of X and Y. For example, “between X and Y” refers to the range of values between the value of X and the value of Y, as well as the value of X and the value of Y.

As used herein, the terms “subject” and “patient” are used interchangeably and refer to an organism, such as a mammal (e.g., a human), that experiences a neurodegenerative disease or disorder (e.g., amyotrophic lateral sclerosis of frontotemporal dementia) and/or contains a gain-of-function C9orf72 variant allele or a repeat expansion of the C9orf72 gene.

As used herein, the terms “neuroinflammatory disease” and “neuroinflammatory disorder” are used interchangeably to refer to any condition that is in some way caused by neuroinflammation. “Neuroinflammation” refers to a range of immune responses in the central nervous system (e.g., in microglia). Neuroinflammation may be brain-derived or result from a systemic inflammatory response.

As used herein, the terms “neurodegenerative disease” and “neurodegenerative disorder” are used interchangeably to refer to any condition that is in some way caused by a loss of function or death of cells of the central nervous system or peripheral nervous system. Exemplary neurodegenerative diseases are Alzheimer's disease, Huntington's disease, amyotrophic lateral sclerosis, frontotemporal dementia, and spinocerebellar ataxias.

As used herein, the term “C9orf72” refers to the gene encoding chromosome, including any native C9orf72 gene from any source. The term encompasses “full-length,” unprocessed C9orf72 as well as any form of C9orf72 that results from processing in the cell. The term also encompasses naturally occurring variants of C9orf72, e.g., splice variants or allelic variants. The nucleic acid sequence of an exemplary C9orf72 gene is shown in European Nucleotide Archive (ENA) Accession No. JN681271.1. The amino acid sequence of an exemplary protein encoded by an C9orf72 gene is shown in UNIPROT™ Accession No. Q96LT7.

As used herein, the terms “treat,” “treated,” and “treating” mean both therapeutic treatment and prophylactic or preventative measures wherein the object is to prevent, ameliorate, or slow down (lessen) an undesired physiological condition, disorder, or disease, or obtain beneficial or desired clinical results. Beneficial or desired clinical results include, but are not limited to, a reduction in a patient's reliance on pharmacological treatments; alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical, cognitive, or behavioral (e.g., depressive behavior or apathy) parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “benefit” and “response” are used interchangeably in the context of a subject undergoing therapy for the treatment of, for example, amyotrophic lateral sclerosis (ALS) or frontotemporal dementia (FTD). For example, clinical benefits in the context of a subject having ALS administered an siRNA molecule or siRNA composition of the disclosure include, without limitation, a reduction in involuntary movements, muscle cramps, weakness, loss of motor control. As a further example, clinical benefits in the context of a subject having FTD administered an siRNA molecule or SIRNA composition of the disclosure include, without limitation, a reduction in memory problems, behavioral problems, and language problems. “Benefit” and “response” are also used interchangeably to refer to, for example, a reduction in wild type C9orf72 transcripts, mutant C9orf72 transcripts, variant C9orf72 transcripts, splice isoforms of C9orf72 transcripts, and/or overexpressed C9orf72 transcripts.

### DETAILED DESCRIPTION

The present disclosure provides compositions of small interfering RNA (siRNA) molecules with sequence homology to a chromosome 9 open reading frame 72 (C9orf72) gene and methods for administering said siRNA molecules to a subject. Furthermore, the siRNA molecules described herein may be composed as branched siRNA structures, such as di-branched, tri-branched, and tetra-branched siRNA structures and may further include specific patterns of chemical modifications (e.g., 2′ ribose modifications or internucleoside linkage modifications) to improve resistance against nuclease enzymes, toxicity profile, and physicochemical properties (e.g., thermostability). Small interfering RNA molecules are short, double-stranded RNA molecules. They are capable of mediating RNA interference (RNAi) by degrading mRNA with a complementary nucleotide sequence, thus preventing the translation of the target gene.

The siRNA molecules of the disclosure may exhibit, for example, robust gene-specific suppression of C9orf72, relative to other human genes.

The siRNA molecules of the disclosure may feature an antisense strand having a nucleic acid sequence that is complementary to a region of a C9orf72 mRNA transcript having the nucleic acid sequence of any one of SEQ ID NOS: 1-384. The degree of complementarity of the antisense strand to the region of the C9orf72 mRNA transcript may be sufficient for the antisense strand to anneal over the full length of the region of the C9orf72 mRNA transcript. For example, the antisense strand may have a nucleic acid sequence that is at least 60% complementary (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary) to the region of the C9orf72 mRNA transcript. In some embodiments, the region of the C9orf72 RNA transcript has the sequence of any one of SEQ ID NOs: 1-192. In some embodiments, the region of the C9orf72 RNA transcript has the sequence of any one of SEQ ID NOs: 193-384.

In some embodiments, the siRNA molecules of the disclosure feature an antisense strand having the nucleic acid sequence of any one of SEQ ID NOs: 769-1152, or a nucleic acid sequence that is at least 60% identical thereto. For example, the siRNA molecules of the disclosure may feature an antisense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 769-1152. In some embodiments, the nucleic acid sequence is any one of SEQ ID NOs: 769-960. In some embodiments, the nucleic acid sequence is any one of SEQ ID NOs: 961-1152.

In some embodiments, the siRNA molecules of the disclosure feature a sense strand having the nucleic acid sequence of any one of SEQ ID NOS: 385-768, or a nucleic acid sequence that is at least 60% identical thereto. For example, the siRNA molecules of the disclosure may feature a sense strand having a nucleic acid sequence that is at least 60% identical (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical) to the nucleic acid sequence of any one of SEQ ID NOs: 385-768. In some embodiments, the nucleic acid sequence is any one of SEQ ID NOs: 385-576. In some embodiments, the nucleic acid sequence is any one of SEQ ID NOs: 577-768.

Exemplary siRNA molecules of the disclosure are those shown in Table 1, below. Table 1 summarizes the antisense strands, sense strands, and corresponding regions of a C9orf72mRNA transcript that are targeted by each antisense strand.

TABLE 1
Nucleotide sequences for gene-specific C9orf72-targeting siRNA

| Gene | | | | | |
|---|---|---|---|---|---|
| region | | Sense | | Antisense | |
| SEQ ID | | SEQ ID | | SEQ ID | |
| NO: | Gene Region | NO: | Sense Sequence | NO: | Antisense Sequence |
| 1 | UCCAGCUGUUG | 385 | CUGUUGCCAAGA | 769 | UCUGUCUUGGCAACA |
| | CCAAGACAGA | | CAGA | | GCUGGA |
| 2 | CAGCUGUUGCC | 386 | GUUGCCAAGACA | 770 | UCUCUGUCUUGGCAA |
| | AAGACAGAGA | | GAGA | | CAGCUG |
| 3 | GCUGUUGCCAA | 387 | UGCCAAGACAGA | 771 | UAUCUCUGUCUUGGC |
| | GACAGAGAUU | | GAUA | | AACAGC |
| 4 | CUGUUGCCAAG | 388 | GCCAAGACAGAG | 772 | UAAUCUCUGUCUUGG |
| | ACAGAGAUUG | | AUUA | | CAACAG |
| 5 | UGUUGCCAAGA | 389 | CCAAGACAGAGAU | 773 | UCAAUCUCUGUCUUG |
| | CAGAGAUUGC | | UGA | | GCAACA |
| 6 | GUUGCCAAGAC | 390 | CAAGACAGAGAUU | 774 | UGCAAUCUCUGUCUU |
| | AGAGAUUGCU | | GCA | | GGCAAC |
| 7 | UUGCCAAGACA | 391 | AAGACAGAGAUU | 775 | UAGCAAUCUCUGUCU |
| | GAGAUUGCUU | | GCUA | | UGGCAA |
| 8 | CCAAGACAGAGA | 392 | ACAGAGAUUGCU | 776 | UUAAAGCAAUCUCUG |
| | UUGCUUUAA | | UUAA | | UCUUGG |
| 9 | ACAGAGAUUGC | 393 | GAUUGCUUUAAG | 777 | UCCACUUAAAGCAAU |
| | UUUAAGUGGC | | UGGA | | CUCUGU |
| 10 | AAUCACCUUUAU | 394 | CCUUUAUUAGCA | 778 | UAGCUGCUAAUAAAG |
| | UAGCAGCUA | | GCUA | | GUGAUU |
| 11 | GGACAAUAUUC | 395 | AUAUUCUUGGUC | 779 | UUAGGACCAAGAAUA |
| | UUGGUCCUAG | | CUAA | | UUGUCC |
| 12 | GACAAUAUUCUU | 396 | UAUUCUUGGUCC | 780 | UCUAGGACCAAGAAU |
| | GGUCCUAGA | | UAGA | | AUUGUC |
| 13 | CAAUAUUCUUG | 397 | UUCUUGGUCCUA | 781 | UCUCUAGGACCAAGA |
| | GUCCUAGAGU | | GAGA | | AUAUUG |
| 14 | AUAUUCUUGGU | 398 | CUUGGUCCUAGA | 782 | UUACUCUAGGACCAA |
| | CCUAGAGUAA | | GUAA | | GAAUAU |
| 15 | UUCUUGGUCCU | 399 | GGUCCUAGAGUA | 783 | UCCUUACUCUAGGAC |
| | AGAGUAAGGC | | AGGA | | CAAGAA |
| 16 | UCUUGGUCCUA | 400 | GUCCUAGAGUAA | 784 | UGCCUUACUCUAGGA |
| | GAGUAAGGCA | | GGCA | | CCAAGA |
| 17 | CUUGGUCCUAG | 401 | UCCUAGAGUAAG | 785 | UUGCCUUACUCUAGG |
| | AGUAAGGCAC | | GCAA | | ACCAAG |
| 18 | GCACAUUUGGG | 402 | UUUGGGCUCCAA | 786 | UUCUUUGGAGCCCAA |
| | CUCCAAAGAC | | AGAA | | AUGUGC |
| 19 | CACAUUUGGGC | 403 | UUGGGCUCCAAA | 787 | UGUCUUUGGAGCCCA |
| | UCCAAAGACA | | GACA | | AAUGUG |
| 20 | ACAUUUGGGCU | 404 | UGGGCUCCAAAG | 788 | UUGUCUUUGGAGCCC |
| | CCAAAGACAG | | ACAA | | AAAUGU |
| 21 | CAUUUGGGCUC | 405 | GGGCUCCAAAGA | 789 | UCUGUCUUUGGAGCC |
| | CAAAGACAGA | | CAGA | | CAAAUG |
| 22 | AGAAAUAACUUU | 406 | UAACUUUUCUUG | 790 | UUGGCAAGAAAAGUU |
| | UCUUGCCAA | | CCAA | | AUUUCU |
| 23 | AAUAACUUUUCU | 407 | CUUUUCUUGCCA | 791 | UGGUUGGCAAGAAAA |
| | UGCCAACCA | | ACCA | | GUUAUU |
| 24 | AUAACUUUUCUU | 408 | UUUUCUUGCCAA | 792 | UUGGUUGGCAAGAAA |
| | GCCAACCAC | | CCAA | | AGUUAU |
| 25 | UAACUUUUCUU | 409 | UUUCUUGCCAAC | 793 | UGUGGUUGGCAAGAA |
| | GCCAACCACA | | CACA | | AAGUUA |
| 26 | UUUUCUUGCCA | 410 | UUGCCAACCACAC | 794 | UGAGUGUGGUUGGC |
| | ACCACACUCU | | UCA | | AAGAAAA |
| 27 | UUUCUUGCCAA | 411 | UGCCAACCACACU | 795 | UAGAGUGUGGUUGG |
| | CCACACUCUA | | CUA | | CAAGAAA |
| 28 | UUCUUGCCAAC | 412 | GCCAACCACACUC | 796 | UUAGAGUGUGGUUG |
| | CACACUCUAA | | UAA | | GCAAGAA |
| 29 | UCUUGCCAACC | 413 | CCAACCACACUCU | 797 | UUUAGAGUGUGGUUG |
| | ACACUCUAAA | | AAA | | GCAAGA |
| 30 | CUUGCCAACCA | 414 | CAACCACACUCUA | 798 | UUUUAGAGUGUGGUU |
| | CACUCUAAAU | | AAA | | GGCAAG |
| 31 | UUGCCAACCACA | 415 | AACCACACUCUAA | 799 | UAUUUAGAGUGUGGU |
| | CUCUAAAUG | | AUA | | UGGCAA |
| 32 | UGCCAACCACAC | 416 | ACCACACUCUAAA | 800 | UCAUUUAGAGUGUGG |
| | UCUAAAUGG | | UGA | | UUGGCA |
| 33 | CAACCACACUCU | 417 | ACACUCUAAAUGG | 801 | UCUCCAUUUAGAGUG |
| | AAAUGGAGA | | AGA | | UGGUUG |
| 34 | AAUCCUUCGAAA | 418 | UUCGAAAUGCAG | 802 | UUCUCUGCAUUUCGA |
| | UGCAGAGAG | | AGAA | | AGGAUU |
| 35 | CCUUCGAAAUG | 419 | GAAAUGCAGAGA | 803 | UCACUCUCUGCAUUU |
| | CAGAGAGUGG | | GUGA | | CGAAGG |
| 36 | AGAGUGGUGCU | 420 | GGUGCUAUAGAU | 804 | UUACAUCUAUAGCAC |
| | AUAGAUGUAA | | GUAA | | CACUCU |
| 37 | GAGUGGUGCUA | 421 | GUGCUAUAGAUG | 805 | UUUACAUCUAUAGCA |
| | UAGAUGUAAA | | UAAA | | CCACUC |
| 38 | UGGUGCUAUAG | 422 | CUAUAGAUGUAAA | 806 | UACUUUACAUCUAUA |
| | AUGUAAAGUU | | GUA | | GCACCA |
| 39 | GUGCUAUAGAU | 423 | AUAGAUGUAAAGU | 807 | UAAACUUUACAUCUA |
| | GUAAAGUUUU | | UUA | | UAGCAC |
| 40 | UUUGUCUUGUC | 424 | CUUGUCUGAAAA | 808 | UCCCUUUUCAGACAA |
| | UGAAAAGGGA | | GGGA | | GACAAA |
| 41 | UCUUGUCUGAA | 425 | UCUGAAAAGGGA | 809 | UCACUCCCUUUUCAG |
| | AAGGGAGUGA | | GUGA | | ACAAGA |
| 42 | GUCUGAAAAGG | 426 | AAAAGGGAGUGA | 810 | UUAAUCACUCCCUUU |
| | GAGUGAUUAU | | UUAA | | UCAGAC |
| 43 | UCUGAAAAGGG | 427 | AAAGGGAGUGAU | 811 | UAUAAUCACUCCCUU |
| | AGUGAUUAUU | | UAUA | | UUCAGA |
| 44 | AAAAGGGAGUG | 428 | GGAGUGAUUAUU | 812 | UAACAAUAAUCACUC |
| | AUUAUUGUUU | | GUUA | | CCUUUU |
| 45 | AUCUUUGAUGG | 429 | UGAUGGAAACUG | 813 | UUUCCAGUUUCCAUC |
| | AAACUGGAAU | | GAAA | | AAAGAU |
| 46 | AUUAUACUUCCA | 430 | ACUUCCACAGACA | 814 | UUCUGUCUGUGGAAG |
| | CAGACAGAA | | GAA | | UAUAAU |
| 47 | UUAUACUUCCAC | 431 | CUUCCACAGACA | 815 | UUUCUGUCUGUGGAA |
| | AGACAGAAC | | GAAA | | GUAUAA |
| 48 | UACUUCCACAGA | 432 | CCACAGACAGAAC | 816 | UAAGUUCUGUCUGUG |
| | CAGAACUUA | | UUA | | GAAGUA |
| 49 | CACAGACAGAAC | 433 | ACAGAACUUAGUU | 817 | UGAAACUAAGUUCUG |
| | UUAGUUUCU | | UCA | | UCUGUG |
| 50 | UAGUUUCUACC | 434 | UCUACCUCCCAC | 818 | UGAAGUGGGAGGUAG |
| | UCCCACUUCA | | UUCA | | AAACUA |
| 51 | AGUUUCUACCU | 435 | CUACCUCCCACU | 819 | UUGAAGUGGGAGGUA |
| | CCCACUUCAU | | UCAA | | GAAACU |
| 52 | CACAUAUAAUCC | 436 | AUAAUCCGGAAAG | 820 | UUCCUUUCCGGAUUA |
| | GGAAAGGAA | | GAA | | UAUGUG |
| 53 | UAUAAUCCGGAA | 437 | UCCGGAAAGGAA | 821 | UUUCUUCCUUUCCGG |
| | AGGAAGAAU | | GAAA | | AUUAUA |
| 54 | AAUCCGGAAAG | 438 | GGAAAGGAAGAA | 822 | UAUAUUCUUCCUUUC |
| | GAAGAAUAUG | | UAUA | | CGGAUU |
| 55 | UCCGGAAAGGA | 439 | AAAGGAAGAAUAU | 823 | UCCAUAUUCUUCCUU |
| | AGAAUAUGGA | | GGA | | UCCGGA |
| 56 | AAGGAAGAAUAU | 440 | AGAAUAUGGAUG | 824 | UAUGCAUCCAUAUUC |
| | GGAUGCAUA | | CAUA | | UUCCUU |
| 57 | GAAGAAUAUGG | 441 | AUAUGGAUGCAU | 825 | UCUUAUGCAUCCAUA |
| | AUGCAUAAGG | | AAGA | | UUCUUC |
| 58 | AGAAUAUGGAU | 442 | AUGGAUGCAUAA | 826 | UUCCUUAUGCAUCCA |
| | GCAUAAGGAA | | GGAA | | UAUUCU |
| 59 | GAAUAUGGAUG | 443 | UGGAUGCAUAAG | 827 | UUUCCUUAUGCAUCC |
| | CAUAAGGAAA | | GAAA | | AUAUUC |
| 60 | AAUAUGGAUGC | 444 | GGAUGCAUAAGG | 828 | UUUUCCUUAUGCAUC |
| | AUAAGGAAAG | | AAAA | | CAUAUU |
| 61 | AUGGAUGCAUA | 445 | UGCAUAAGGAAA | 829 | UGUCUUUCCUUAUGC |
| | AGGAAAGACA | | GACA | | AUCCAU |
| 62 | UGGAUGCAUAA | 446 | GCAUAAGGAAAGA | 830 | UUGUCUUUCCUUAUG |
| | GGAAAGACAA | | CAA | | CAUCCA |
| 63 | GGAUGCAUAAG | 447 | CAUAAGGAAAGAC | 831 | UUUGUCUUUCCUUAU |
| | GAAAGACAAG | | AAA | | GCAUCC |
| 64 | AGGAAAGACAAG | 448 | AGACAAGAAAAUG | 832 | UGACAUUUUCUUGUC |
| | AAAAUGUCC | | UCA | | UUUCCU |
| 65 | AAGACAAGAAAA | 449 | AAGAAAAUGUCCA | 833 | UUCUGGACAUUUUCU |
| | UGUCCAGAA | | GAA | | UGUCUU |
| 66 | UAUCUUAGAAG | 450 | UAGAAGGCACAG | 834 | UUCUCUGUGCCUUCU |
| | GCACAGAGAG | | AGAA | | AAGAUA |
| 67 | AGAGAGAAUGG | 451 | GAAUGGAAGAUC | 835 | UCCUGAUCUUCCAUU |
| | AAGAUCAGGG | | AGGA | | CUCUCU |
| 68 | GAGAAUGGAAG | 452 | UGGAAGAUCAGG | 836 | UGACCCUGAUCUUCC |
| | AUCAGGGUCA | | GUCA | | AUUCUC |
| 69 | AGAAUGGAAGA | 453 | GGAAGAUCAGGG | 837 | UUGACCCUGAUCUUC |
| | UCAGGGUCAG | | UCAA | | CAUUCU |
| 70 | GAAUGGAAGAU | 454 | GAAGAUCAGGGU | 838 | UCUGACCCUGAUCUU |
| | CAGGGUCAGA | | CAGA | | CCAUUC |
| 71 | AAUGGAAGAUCA | 455 | AAGAUCAGGGUC | 839 | UUCUGACCCUGAUCU |
| | GGGUCAGAG | | AGAA | | UCCAUU |
| 72 | AUGGAAGAUCA | 456 | AGAUCAGGGUCA | 840 | UCUCUGACCCUGAUC |
| | GGGUCAGAGU | | GAGA | | UUCCAU |
| 73 | UGGAAGAUCAG | 457 | GAUCAGGGUCAG | 841 | UACUCUGACCCUGAU |
| | GGUCAGAGUA | | AGUA | | CUUCCA |
| 74 | GGAAGAUCAGG | 458 | AUCAGGGUCAGA | 842 | UUACUCUGACCCUGA |
| | GUCAGAGUAU | | GUAA | | UCUUCC |
| 75 | GAAGAUCAGGG | 459 | UCAGGGUCAGAG | 843 | UAUACUCUGACCCUG |
| | UCAGAGUAUU | | UAUA | | AUCUUC |
| 76 | GAUCAGGGUCA | 460 | GGGUCAGAGUAU | 844 | UAUAAUACUCUGACC |
| | GAGUAUUAUU | | UAUA | | CUGAUC |
| 77 | CAGGGUCAGAG | 461 | UCAGAGUAUUAU | 845 | UGGAAUAAUACUCUG |
| | UAUUAUUCCA | | UCCA | | ACCCUG |
| 78 | UGGAGAAGUGA | 462 | AAGUGAUUCCUG | 846 | UUUACAGGAAUCACU |
| | UUCCUGUAAU | | UAAA | | UCUCCA |
| 79 | CUGUAAUGGAA | 463 | AUGGAACUGCUU | 847 | UUGAAAGCAGUUCCA |
| | CUGCUUUCAU | | UCAA | | UUACAG |
| 80 | AUGGAACUGCU | 464 | ACUGCUUUCAUC | 848 | UAUAGAUGAAAGCAG |
| | UUCAUCUAUG | | UAUA | | UUCCAU |
| 81 | UUCAUCUAUGAA | 465 | CUAUGAAAUCACA | 849 | UUGUGUGAUUUCAUA |
| | AUCACACAG | | CAA | | GAUGAA |
| 82 | CUAUGAAAUCAC | 466 | AAAUCACACAGUG | 850 | UAACACUGUGUGAUU |
| | ACAGUGUUC | | UUA | | UCAUAG |
| 83 | UCCUGAAGAAAU | 467 | AAGAAAUAGAUAU | 851 | UCUAUAUCUAUUUCU |
| | AGAUAUAGC | | AGA | | UCAGGA |
| 84 | AAUAGAUAUAGC | 468 | AUAUAGCUGAUAC | 852 | UCUGUAUCAGCUAUA |
| | UGAUACAGU | | AGA | | UCUAUU |
| 85 | CUGAUACAGUA | 469 | ACAGUACUCAAUG | 853 | UAUCAUUGAGUACUG |
| | CUCAAUGAUG | | AUA | | UAUCAG |
| 86 | UGAAGGCUUUC | 470 | GCUUUCUUCUCA | 854 | UCAUUGAGAAGAAAG |
| | UUCUCAAUGC | | AUGA | | CCUUCA |
| 87 | GAAGGCUUUCU | 471 | CUUUCUUCUCAA | 855 | UGCAUUGAGAAGAAA |
| | UCUCAAUGCC | | UGCA | | GCCUUC |
| 88 | AAGGCUUUCUU | 472 | UUUCUUCUCAAU | 856 | UGGCAUUGAGAAGAA |
| | CUCAAUGCCA | | GCCA | | AGCCUU |
| 89 | CUUCUCAAUGC | 473 | CAAUGCCAUCAG | 857 | UGAGCUGAUGGCAUU |
| | CAUCAGCUCA | | CUCA | | GAGAAG |
| 90 | UUCUCAAUGCC | 474 | AAUGCCAUCAGC | 858 | UUGAGCUGAUGGCAU |
| | AUCAGCUCAC | | UCAA | | UGAGAA |
| 91 | UCUCAAUGCCA | 475 | AUGCCAUCAGCU | 859 | UGUGAGCUGAUGGCA |
| | UCAGCUCACA | | CACA | | UUGAGA |
| 92 | UCAAUGCCAUCA | 476 | GCCAUCAGCUCA | 860 | UGUGUGAGCUGAUG |
| | GCUCACACU | | CACA | | GCAUUGA |
| 93 | CAAACCUGUGG | 477 | CUGUGGCUGUUC | 861 | UACGGAACAGCCACA |
| | CUGUUCCGUU | | CGUA | | GGUUUG |
| 94 | ACCUGUGGCUG | 478 | UGGCUGUUCCGU | 862 | UACAACGGAACAGCC |
| | UUCCGUUGUA | | UGUA | | ACAGGU |
| 95 | CUGUGGCUGUU | 479 | GCUGUUCCGUUG | 863 | UCUACAACGGAACAG |
| | CCGUUGUAGU | | UAGA | | CCACAG |
| 96 | GUUGUAGUAGG | 480 | AGUAGGUAGCAG | 864 | UGCACUGCUACCUAC |
| | UAGCAGUGCA | | UGCA | | UACAAC |
| 97 | GGUAGCAGUGC | 48 | CAGUGCAGAGAA | 865 | UACUUUCUCUGCACU |
| | AGAGAAAGUA | | AGUA | | GCUACC |
| 98 | AGCAGUGCAGA | 482 | UGCAGAGAAAGU | 866 | UUUUACUUUCUCUGC |
| | GAAAGUAAAU | | AAAA | | ACUGCU |
| 99 | GCAGUGCAGAG | 483 | GCAGAGAAAGUAA | 867 | UAUUUACUUUCUCUG |
| | AAAGUAAAUA | | AUA | | CACUGC |
| 100 | GCAGAGAAAGU | 484 | GAAAGUAAAUAAG | 868 | UAUCUUAUUUACUUU |
| | AAAUAAGAUA | | AUA | | CUCUGC |
| 101 | GAUAGUCAGAA | 485 | UCAGAACAUUAUG | 869 | UGGCAUAAUGUUCUG |
| | CAUUAUGCCU | | CCA | | ACUAUC |
| 102 | UGAAGCAGAAU | 486 | CAGAAUCAUCAUU | 870 | UUAAAUGAUGAUUCU |
| | CAUCAUUUAA | | UAA | | GCUUCA |
| 103 | GAAGCAGAAUCA | 487 | AGAAUCAUCAUUU | 871 | UUUAAAUGAUGAUUC |
| | UCAUUUAAA | | AAA | | UGCUUC |
| 104 | CUGCUAAAGGA | 488 | AAAGGAUUCAACU | 872 | UCCAGUUGAAUCCUU |
| | UUCAACUGGA | | GGA | | UAGCAG |
| 105 | UUCCGGCAAGU | 489 | GCAAGUCAUGUA | 873 | UGCAUACAUGACUUG |
| | CAUGUAUGCU | | UGCA | | CCGGAA |
| 106 | CUCCAUAUCCCA | 490 | UAUCCCACCACAC | 874 | UGUGUGUGGUGGGA |
| | CCACACACA | | ACA | | UAUGGAG |
| 107 | UGCCACCCUGU | 491 | CCCUGUCAUGAA | 875 | UAUGUUCAUGACAGG |
| | CAUGAACAUA | | CAUA | | GUGGCA |
| 108 | GCCACCCUGUC | 492 | CCUGUCAUGAAC | 876 | UUAUGUUCAUGACAG |
| | AUGAACAUAU | | AUAA | | GGUGGC |
| 109 | CCCUGUCAUGA | 493 | UCAUGAACAUAUU | 877 | UUAAAUAUGUUCAUG |
| | ACAUAUUUAU | | UAA | | ACAGGG |
| 110 | UUAUAAUCAGC | 494 | AUCAGCGUAGAU | 878 | UUGUAUCUACGCUGA |
| | GUAGAUACAU | | ACAA | | UUAUAA |
| 111 | AAUCAGCGUAG | 495 | GCGUAGAUACAU | 879 | UCUCAUGUAUCUACG |
| | AUACAUGAGA | | GAGA | | CUGAUU |
| 112 | AUGGCUCAGGA | 496 | UCAGGAUACGAU | 880 | UAUGAUCGUAUCCUG |
| | UACGAUCAUC | | CAUA | | AGCCAU |
| 113 | CGAUCAUCUACA | 497 | AUCUACACUGAC | 881 | UUUCGUCAGUGUAGA |
| | CUGACGAAA | | GAAA | | UGAUCG |
| 114 | UGACGAAAGCU | 498 | AAAGCUUUACUCC | 882 | UCAGGAGUAAAGCUU |
| | UUACUCCUGA | | UGA | | UCGUCA |
| 115 | CUUUACUCCUG | 499 | CUCCUGAUUUGA | 883 | UUAUUCAAAUCAGGA |
| | AUUUGAAUAU | | AUAA | | GUAAAG |
| 116 | UUUCAAGAUGU | 500 | AGAUGUCUUACA | 884 | UCUGUGUAAGACAUC |
| | CUUACACAGA | | CAGA | | UUGAAA |
| 117 | AUGUCUUACACA | 501 | UUACACAGAGACA | 885 | UAGUGUCUCUGUGUA |
| | GAGACACUC | | CUA | | AGACAU |
| 118 | UGUCUUACACA | 502 | UACACAGAGACAC | 886 | UGAGUGUCUCUGUGU |
| | GAGACACUCU | | UCA | | AAGACA |
| 119 | GUCUUACACAG | 503 | ACACAGAGACACU | 887 | UAGAGUGUCUCUGUG |
| | AGACACUCUA | | CUA | | UAAGAC |
| 120 | UCUUACACAGA | 504 | CACAGAGACACUC | 888 | UUAGAGUGUCUCUGU |
| | GACACUCUAG | | UAA | | GUAAGA |
| 121 | CUUACACAGAGA | 505 | ACAGAGACACUCU | 889 | UCUAGAGUGUCUCUG |
| | CACUCUAGU | | AGA | | UGUAAG |
| 122 | UUACACAGAGAC | 506 | CAGAGACACUCUA | 890 | UACUAGAGUGUCUCU |
| | ACUCUAGUG | | GUA | | GUGUAA |
| 123 | UACACAGAGACA | 507 | AGAGACACUCUA | 891 | UCACUAGAGUGUCUC |
| | CUCUAGUGA | | GUGA | | UGUGUA |
| 124 | ACACAGAGACAC | 508 | GAGACACUCUAG | 892 | UUCACUAGAGUGUCU |
| | UCUAGUGAA | | UGAA | | CUGUGU |
| 125 | CACAGAGACACU | 509 | AGACACUCUAGU | 893 | UUUCACUAGAGUGUC |
| | CUAGUGAAA | | GAAA | | UCUGUG |
| 126 | ACAGAGACACUC | 510 | GACACUCUAGUG | 894 | UUUUCACUAGAGUGU |
| | UAGUGAAAG | | AAAA | | CUCUGU |
| 127 | CAGAAGUACUU | 511 | GUACUUUCCUUG | 895 | UGUGCAAGGAAAGUA |
| | UCCUUGCACA | | CACA | | CUUCUG |
| 128 | AGUACUUUCCU | 512 | UUUCCUUGCACA | 896 | UAACUGUGCAAGGAA |
| | UGCACAGUUU | | GUUA | | AGUACU |
| 129 | CCUUCACAGAAA | 513 | ACAGAAAAGCCUU | 897 | UUCAAGGCUUUUCUG |
| | AGCCUUGAC | | GAA | | UGAAGG |
| 130 | CACAGAAAAGCC | 514 | AAAAGCCUUGACA | 898 | UAGUGUCAAGGCUUU |
| | UUGACACUA | | CUA | | UCUGUG |
| 131 | ACAGAAAAGCCU | 515 | AAAGCCUUGACAC | 899 | UUAGUGUCAAGGCUU |
| | UGACACUAA | | UAA | | UUCUGU |
| 132 | CAGAAAAGCCUU | 516 | AAGCCUUGACAC | 900 | UUUAGUGUCAAGGCU |
| | GACACUAAU | | UAAA | | UUUCUG |
| 133 | ACGAUACGCAG | 517 | ACGCAGAAGGGA | 901 | UUUUUCCCUUCUGCG |
| | AAGGGAAAAA | | AAAA | | UAUCGU |
| 134 | CGAUACGCAGA | 518 | CGCAGAAGGGAA | 902 | UUUUUUCCCUUCUGC |
| | AGGGAAAAAA | | AAAA | | GUAUCG |
| 135 | GACCUUGAUUU | 519 | UGAUUUAACAGCA | 903 | UUCUGCUGUUAAAUC |
| | AACAGCAGAG | | GAA | | AAGGUC |
| 136 | ACCUUGAUUUAA | 520 | GAUUUAACAGCA | 904 | UCUCUGCUGUUAAAU |
| | CAGCAGAGG | | GAGA | | CAAGGU |
| 137 | CCUUGAUUUAA | 521 | AUUUAACAGCAGA | 905 | UCCUCUGCUGUUAAA |
| | CAGCAGAGGG | | GGA | | UCAAGG |
| 138 | CUUGAUUUAACA | 522 | UUUAACAGCAGA | 906 | UCCCUCUGCUGUUAA |
| | GCAGAGGGC | | GGGA | | AUCAAG |
| 139 | UUGAUUUAACA | 523 | UUAACAGCAGAG | 907 | UGCCCUCUGCUGUUA |
| | GCAGAGGGCG | | GGCA | | AAUCAA |
| 140 | UGAUUUAACAG | 524 | UAACAGCAGAGG | 908 | UCGCCCUCUGCUGUU |
| | CAGAGGGCGA | | GCGA | | AAAUCA |
| 141 | AUUUAACAGCAG | 525 | ACAGCAGAGGGC | 909 | UAUCGCCCUCUGCUG |
| | AGGGCGAUC | | GAUA | | UUAAAU |
| 142 | UUUAACAGCAGA | 526 | CAGCAGAGGGCG | 910 | UGAUCGCCCUCUGCU |
| | GGGCGAUCU | | AUCA | | GUUAAA |
| 143 | UAACAGCAGAG | 527 | GCAGAGGGCGAU | 911 | UAAGAUCGCCCUCUG |
| | GGCGAUCUUA | | CUUA | | CUGUUA |
| 144 | AACAGCAGAGG | 528 | CAGAGGGCGAUC | 912 | UUAAGAUCGCCCUCU |
| | GCGAUCUUAA | | UUAA | | GCUGUU |
| 145 | CAGCAGAGGGC | 529 | GAGGGCGAUCUU | 913 | UGUUAAGAUCGCCCU |
| | GAUCUUAACA | | AACA | | CUGCUG |
| 146 | AGAGGGCGAUC | 530 | GCGAUCUUAACA | 914 | UUUAUGUUAAGAUCG |
| | UUAACAUAAU | | UAAA | | CCCUCU |
| 147 | GAGGGCGAUCU | 531 | CGAUCUUAACAUA | 915 | UAUUAUGUUAAGAUC |
| | UAACAUAAUA | | AUA | | GCCCUC |
| 148 | AGGGCGAUCUU | 532 | GAUCUUAACAUAA | 916 | UUAUUAUGUUAAGAU |
| | AACAUAAUAA | | UAA | | CGCCCU |
| 149 | GGGCGAUCUUA | 533 | AUCUUAACAUAAU | 917 | UUUAUUAUGUUAAGA |
| | ACAUAAUAAU | | AAA | | UCGCCC |
| 150 | CGAUCUUAACAU | 534 | UUAACAUAAUAAU | 918 | UCCAUUAUUAUGUUA |
| | AAUAAUGGC | | GGA | | AGAUCG |
| 151 | GAUCUUAACAUA | 535 | UAACAUAAUAAUG | 919 | UGCCAUUAUUAUGUU |
| | AUAAUGGCU | | GCA | | AAGAUC |
| 152 | UCUUAACAUAAU | 536 | ACAUAAUAAUGGC | 920 | UGAGCCAUUAUUAUG |
| | AAUGGCUCU | | UCA | | UUAAGA |
| 153 | CUUAACAUAAUA | 537 | CAUAAUAAUGGCU | 921 | UAGAGCCAUUAUUAU |
| | AUGGCUCUG | | CUA | | GUUAAG |
| 154 | GGCUCUGGCUG | 538 | UGGCUGAGAAAA | 922 | UUAAUUUUCUCAGCC |
| | AGAAAAUUAA | | UUAA | | AGAGCC |
| 155 | CUCUGGCUGAG | 539 | GCUGAGAAAAUUA | 923 | UUUUAAUUUUCUCAG |
| | AAAAUUAAAC | | AAA | | CCAGAG |
| 156 | UAAACCAGGCC | 540 | CAGGCCUACACU | 924 | UAAGAGUGUAGGCCU |
| | UACACUCUUU | | CUUA | | GGUUUA |
| 157 | UGGAAGACCUU | 541 | GACCUUUCUACA | 925 | UUAGUGUAGAAAGGU |
| | UCUACACUAG | | CUAA | | CUUCCA |
| 158 | GGAAGACCUUU | 542 | ACCUUUCUACACU | 926 | UCUAGUGUAGAAAGG |
| | CUACACUAGU | | AGA | | UCUUCC |
| 159 | GAAGACCUUUC | 543 | CCUUUCUACACUA | 927 | UACUAGUGUAGAAAG |
| | UACACUAGUG | | GUA | | GUCUUC |
| 160 | AAGACCUUUCUA | 544 | CUUUCUACACUA | 928 | UCACUAGUGUAGAAA |
| | CACUAGUGU | | GUGA | | GGUCUU |
| 161 | UGCAAGAACGA | 545 | GAACGAGAUGUU | 929 | UUAGAACAUCUCGUU |
| | GAUGUUCUAA | | CUAA | | CUUGCA |
| 162 | GUGUAACUUAA | 546 | ACUUAAUAAGCCU | 930 | UAUAGGCUUAUUAAG |
| | UAAGCCUAUU | | AUA | | UUACAC |
| 163 | UAACUUAAUAAG | 547 | UAAUAAGCCUAUU | 931 | UGGAAUAGGCUUAUU |
| | CCUAUUCCA | | CCA | | AAGUUA |
| 164 | UAAUAAGCCUAU | 548 | AGCCUAUUCCAU | 932 | UGUGAUGGAAUAGGC |
| | UCCAUCACA | | CACA | | UUAUUA |
| 165 | CAGCAAUUGCA | 549 | AUUGCAGUUAAG | 933 | UUUACUUAACUGCAA |
| | GUUAAGUAAG | | UAAA | | UUGCUG |
| 166 | UGCAGUUAAGU | 550 | UUAAGUAAGUUAC | 934 | UGUGUAACUUACUUA |
| | AAGUUACACU | | ACA | | ACUGCA |
| 167 | GUAAGUUACAC | 551 | UUACACUACAGUU | 935 | UAGAACUGUAGUGUA |
| | UACAGUUCUC | | CUA | | ACUUAC |
| 168 | CAGGUGCAUCA | 552 | GCAUCAUUACAUU | 936 | UCCAAUGUAAUGAUG |
| | UUACAUUGGG | | GGA | | CACCUG |
| 169 | UGCUUUUGGGA | 553 | UUGGGAUACAGA | 937 | UAGGUCUGUAUCCCA |
| | UACAGACCUA | | CCUA | | AAAGCA |
| 170 | GCUUUUGGGAU | 554 | UGGGAUACAGAC | 938 | UUAGGUCUGUAUCCC |
| | ACAGACCUAU | | CUAA | | AAAAGC |
| 171 | UUUUGGGAUAC | 555 | GGAUACAGACCU | 939 | UCAUAGGUCUGUAUC |
| | AGACCUAUGU | | AUGA | | CCAAAA |
| 172 | UUGGGAUACAG | 556 | AUACAGACCUAUG | 940 | UAACAUAGGUCUGUA |
| | ACCUAUGUUU | | UUA | | UCCCAA |
| 173 | UGGGAUACAGA | 557 | UACAGACCUAUG | 941 | UAAACAUAGGUCUGU |
| | CCUAUGUUUA | | UUUA | | AUCCCA |
| 174 | UACAGACCUAU | 558 | ACCUAUGUUUACA | 942 | UAUUGUAAACAUAGG |
| | GUUUACAAUA | | AUA | | UCUGUA |
| 175 | CAGACCUAUGU | 559 | CUAUGUUUACAAU | 943 | UAUAUUGUAAACAUA |
| | UUACAAUAUA | | AUA | | GGUCUG |
| 176 | AUACAUGAUUCA | 560 | UGAUUCAUGGUU | 944 | UGUAAACCAUGAAUC |
| | UGGUUUACA | | UACA | | AUGUAU |
| 177 | GUUGACUUUCU | 561 | CUUUCUAUCUUU | 945 | UCCAAAAGAUAGAAA |
| | AUCUUUUGGC | | UGGA | | GUCAAC |
| 178 | AUUCCACAGAAA | 562 | ACAGAAAGAAAGU | 946 | UUCACUUUCUUUCUG |
| | GAAAGUGAG | | GAA | | UGGAAU |
| 179 | UGAUCAAGCAG | 563 | AAGCAGAUGUUU | 947 | UAUUAAACAUCUGCU |
| | AUGUUUAAUU | | AAUA | | UGAUCA |
| 180 | CAAGCAGAUGU | 564 | AGAUGUUUAAUU | 948 | UUCCAAUUAAACAUC |
| | UUAAUUGGAA | | GGAA | | UGCUUG |
| 181 | CCCUGGGAUUC | 565 | GGAUUCAGUCUG | 949 | UCUACAGACUGAAUC |
| | AGUCUGUAGA | | UAGA | | CCAGGG |
| 182 | UUCAGUCUGUA | 566 | UCUGUAGAAAUG | 950 | UAGACAUUUCUACAG |
| | GAAAUGUCUA | | UCUA | | ACUGAA |
| 183 | UCCUGGUGAAC | 567 | GUGAACCACAGU | 951 | UCUAACUGUGGUUCA |
| | CACAGUUAGG | | UAGA | | CCAGGA |
| 184 | UAUGUAGUUGA | 568 | AGUUGAGCUCUG | 952 | UUUACAGAGCUCAAC |
| | GCUCUGUAAA | | UAAA | | UACAUA |
| 185 | AUGUAGUUGAG | 569 | GUUGAGCUCUGU | 953 | UUUUACAGAGCUCAA |
| | CUCUGUAAAA | | AAAA | | CUACAU |
| 186 | GUAGUUGAGCU | 570 | UGAGCUCUGUAA | 954 | UCUUUUACAGAGCUC |
| | CUGUAAAAGG | | AAGA | | AACUAC |
| 187 | AGUUGAGCUCU | 571 | AGCUCUGUAAAA | 955 | UUCCUUUUACAGAGC |
| | GUAAAAGGAA | | GGAA | | UCAACU |
| 188 | GUUGAGCUCUG | 572 | GCUCUGUAAAAG | 956 | UUUCCUUUUACAGAG |
| | UAAAAGGAAA | | GAAA | | CUCAAC |
| 189 | UUGAGCCAAAU | 573 | CCAAAUUGAAAUG | 957 | UCACAUUUCAAUUUG |
| | UGAAAUGUGC | | UGA | | GCUCAA |
| 190 | UGUGCACCUCC | 574 | ACCUCCUGUGCC | 958 | UAAAGGCACAGGAGG |
| | UGUGCCUUUU | | UUUA | | UGCACA |
| 191 | UUCAGAUUUCA | 575 | AUUUCACUGGUC | 959 | UACUGACCAGUGAAA |
| | CUGGUCAGUC | | AGUA | | UCUGAA |
| 192 | UCUUACCAUGU | 576 | CCAUGUACCUGC | 960 | UAAAGCAGGUACAUG |
| | ACCUGCUUUG | | UUUA | | GUAAGA |
| 193 | GUCCCGCUAGG | 577 | GCUAGGAAAGAG | 961 | UCCUCUCUUUCCUAG |
| | AAAGAGAGGU | | AGGA | | CGGGAC |
| 194 | GUCAAACAGCG | 578 | ACAGCGACAAGU | 962 | UGGAACUUGUCGCUG |
| | ACAAGUUCCG | | UCCA | | UUUGAC |
| 195 | UCAAACAGCGAC | 579 | CAGCGACAAGUU | 963 | UCGGAACUUGUCGCU |
| | AAGUUCCGC | | CCGA | | GUUUGA |
| 196 | CAGCGACAAGU | 580 | ACAAGUUCCGCC | 964 | UGUGGGCGGAACUU |
| | UCCGCCCACG | | CACA | | GUCGCUG |
| 197 | CGACAAGUUCC | 581 | AGUUCCGCCCAC | 965 | UUACGUGGGCGGAAC |
| | GCCCACGUAA | | GUAA | | UUGUCG |
| 198 | GACAAGUUCCG | 582 | GUUCCGCCCACG | 966 | UUUACGUGGGCGGAA |
| | CCCACGUAAA | | UAAA | | CUUGUC |
| 199 | AAGUUCCGCCC | 583 | CCGCCCACGUAA | 967 | UCUUUUACGUGGGCG |
| | ACGUAAAAGA | | AAGA | | GAACUU |
| 200 | UUCCGCCCACG | 584 | CCCACGUAAAAGA | 968 | UCAUCUUUUACGUGG |
| | UAAAAGAUGA | | UGA | | GCGGAA |
| 201 | UCCGCCCACGU | 585 | CCACGUAAAAGAU | 969 | UUCAUCUUUUACGUG |
| | AAAAGAUGAC | | GAA | | GGCGGA |
| 202 | CCGCCCACGUA | 586 | CACGUAAAAGAUG | 970 | UGUCAUCUUUUACGU |
| | AAAGAUGACG | | ACA | | GGGCGG |
| 203 | CGCCCACGUAA | 587 | ACGUAAAAGAUGA | 971 | UCGUCAUCUUUUACG |
| | AAGAUGACGC | | CGA | | UGGGCG |
| 204 | CCACGUAAAAGA | 588 | UAAAAGAUGACGC | 972 | UAAGCGUCAUCUUUU |
| | UGACGCUUG | | UUA | | ACGUGG |
| 205 | GUGUGUCAGCC | 589 | UCAGCCGUCCCU | 973 | UAGCAGGGACGGCUG |
| | GUCCCUGCUG | | GCUA | | ACACAC |
| 206 | UGCUGCCCGGU | 590 | CCCGGUUGCUUC | 974 | UAGAGAAGCAACCGG |
| | UGCUUCUCUU | | UCUA | | GCAGCA |
| 207 | GUCUAGCAAGA | 591 | GCAAGAGCAGGU | 975 | UCACACCUGCUCUUG |
| | GCAGGUGUGG | | GUGA | | CUAGAC |
| 208 | CAAGAGCAGGU | 592 | GCAGGUGUGGGU | 976 | UUAAACCCACACCUG |
| | GUGGGUUUAG | | UUAA | | CUCUUG |
| 209 | AGGUGUGGGUU | 593 | UGGGUUUAGGAG | 977 | UCACCUCCUAAACCC |
| | UAGGAGGUGU | | GUGA | | ACACCU |
| 210 | GGUGUGGGUUU | 594 | GGGUUUAGGAGG | 978 | UACACCUCCUAAACC |
| | AGGAGGUGUG | | UGUA | | CACACC |
| 211 | GUGUGGGUUUA | 595 | GGUUUAGGAGGU | 979 | UCACACCUCCUAAAC |
| | GGAGGUGUGU | | GUGA | | CCACAC |
| 212 | GUGGGUUUAGG | 596 | UUUAGGAGGUGU | 980 | UCACACACCUCCUAA |
| | AGGUGUGUGU | | GUGA | | ACCCAC |
| 213 | UGGGUUUAGGA | 597 | UUAGGAGGUGUG | 981 | UACACACACCUCCUA |
| | GGUGUGUGUU | | UGUA | | AACCCA |
| 214 | GGGUUUAGGAG | 598 | UAGGAGGUGUGU | 982 | UAACACACACCUCCU |
| | GUGUGUGUUU | | GUUA | | AAACCC |
| 215 | GUUUAGGAGGU | 599 | GGAGGUGUGUGU | 983 | UAAAACACACACCUC |
| | GUGUGUUUUU | | UUUA | | CUAAAC |
| 216 | UUUAGGAGGUG | 600 | GAGGUGUGUGUU | 984 | UAAAAACACACACCU |
| | UGUGUUUUUG | | UUUA | | CCUAAA |
| 217 | UUAGGAGGUGU | 601 | AGGUGUGUGUUU | 985 | UCAAAAACACACACC |
| | GUGUUUUUGU | | UUGA | | UCCUAA |
| 218 | GUUUUUGUUUU | 602 | UGUUUUUCCCAC | 986 | UAGGGUGGGAAAAAC |
| | UCCCACCCUC | | CCUA | | AAAAAC |
| 219 | UCGCUGAGGGU | 603 | GAGGGUGAACAA | 987 | UUUCUUGUUCACCCU |
| | GAACAAGAAA | | GAAA | | CAGCGA |
| 220 | CGCUGAGGGUG | 604 | AGGGUGAACAAG | 988 | UUUUCUUGUUCACCC |
| | AACAAGAAAA | | AAAA | | UCAGCG |
| 221 | GAGGGUGAACA | 605 | UGAACAAGAAAAG | 989 | UGUCUUUUCUUGUUC |
| | AGAAAAGACC | | ACA | | ACCCUC |
| 222 | GGUGAACAAGA | 606 | ACAAGAAAAGACC | 990 | UCAGGUCUUUUCUUG |
| | AAAGACCUGA | | UGA | | UUCACC |
| 223 | GUGAACAAGAAA | 607 | CAAGAAAAGACCU | 991 | UUCAGGUCUUUUCUU |
| | AGACCUGAU | | GAA | | GUUCAC |
| 224 | UGAACAAGAAAA | 608 | AAGAAAAGACCUG | 992 | UAUCAGGUCUUUUCU |
| | GACCUGAUA | | AUA | | UGUUCA |
| 225 | AACAAGAAAAGA | 609 | GAAAAGACCUGAU | 993 | UUUAUCAGGUCUUUU |
| | CCUGAUAAA | | AAA | | CUUGUU |
| 226 | AGACCUGAUAAA | 610 | UGAUAAAGAUUAA | 994 | UGGUUAAUCUUUAUC |
| | GAUUAACCA | | CCA | | AGGUCU |
| 227 | GACCUGAUAAA | 611 | GAUAAAGAUUAAC | 995 | UUGGUUAAUCUUUAU |
| | GAUUAACCAG | | CAA | | CAGGUC |
| 228 | ACCUGAUAAAGA | 612 | AUAAAGAUUAACC | 996 | UCUGGUUAAUCUUUA |
| | UUAACCAGA | | AGA | | UCAGGU |
| 229 | CUGAUAAAGAUU | 613 | AAAGAUUAACCAG | 997 | UUUCUGGUUAAUCUU |
| | AACCAGAAG | | AAA | | UAUCAG |
| 230 | UGAUAAAGAUUA | 614 | AAGAUUAACCAGA | 998 | UCUUCUGGUUAAUCU |
| | ACCAGAAGA | | AGA | | UUAUCA |
| 231 | GAUUAACCAGAA | 615 | ACCAGAAGAAAAC | 999 | UUUGUUUUCUUCUGG |
| | GAAAACAAG | | AAA | | UUAAUC |
| 232 | AUUAACCAGAAG | 616 | CCAGAAGAAAACA | 1000 | UCUUGUUUUCUUCUG |
| | AAAACAAGG | | AGA | | GUUAAU |
| 233 | UUAACCAGAAGA | 617 | CAGAAGAAAACAA | 1001 | UCCUUGUUUUCUUCU |
| | AAACAAGGA | | GGA | | GGUUAA |
| 234 | ACAAGGAGGGA | 618 | GAGGGAAACAAC | 1002 | UGCGGUUGUUUCCCU |
| | AACAACCGCA | | CGCA | | CCUUGU |
| 235 | CAAGGAGGGAA | 619 | AGGGAAACAACC | 1003 | UUGCGGUUGUUUCCC |
| | ACAACCGCAG | | GCAA | | UCCUUG |
| 236 | GGAGGGAAACA | 620 | GAAACAACCGCAG | 1004 | UGGCUGCGGUUGUU |
| | ACCGCAGCCU | | CCA | | UCCCUCC |
| 237 | GGGAAACAACC | 621 | ACAACCGCAGCC | 1005 | UACAGGCUGCGGUUG |
| | GCAGCCUGUA | | UGUA | | UUUCCC |
| 238 | CAGGAGUCGCG | 622 | GUCGCGCGCUAG | 1006 | UCCCCUAGCGCGCGA |
| | CGCUAGGGGC | | GGGA | | CUCCUG |
| 239 | GGGGCCGGGGC | 623 | CGGGGCCGGGGC | 1007 | UCCGGCCCCGGCCCC |
| | CGGGGCCGGG | | CGGA | | GGCCCC |
| 240 | GGGCCGGGGCC | 624 | GGGGCCGGGGCC | 1008 | UCCCGGCCCCGGCCC |
| | GGGGCCGGGG | | GGGA | | CGGCCC |
| 241 | GGCCGGGGCCG | 625 | GGGCCGGGGCCG | 1009 | UCCCCGGCCCCGGCC |
| | GGGCCGGGGC | | GGGA | | CCGGCC |
| 242 | GCCGGGGCCGG | 626 | GGCCGGGGCCGG | 1010 | UGCCCCGGCCCCGG |
| | GGCCGGGGCC | | GGCA | | CCCCGGC |
| 243 | CCGGGGCCGGG | 627 | GCCGGGGCCGGG | 1011 | UGGCCCCGGCCCCG |
| | GCCGGGGCCG | | GCCA | | GCCCCGG |
| 244 | CGGGGCCGGGG | 628 | CCGGGGCCGGGG | 1012 | UCGGCCCCGGCCCC |
| | CCGGGGCCGG | | CCGA | | GGCCCCG |
| 245 | CUCAGAGCUCG | 629 | AGCUCGACGCAU | 1013 | UAAAAUGCGUCGAGC |
| | ACGCAUUUUU | | UUUA | | UCUGAG |
| 246 | UCAGAGCUCGA | 630 | GCUCGACGCAUU | 1014 | UAAAAAUGCGUCGAG |
| | CGCAUUUUUA | | UUUA | | CUCUGA |
| 247 | UCGACGCAUUU | 631 | GCAUUUUUACUU | 1015 | UGGAAAGUAAAAAUG |
| | UUACUUUCCC | | UCCA | | CGUCGA |
| 248 | GACGCAUUUUU | 632 | AUUUUUACUUUC | 1016 | UAGGGAAAGUAAAAA |
| | ACUUUCCCUC | | CCUA | | UGCGUC |
| 249 | UUCCCUCUCAU | 633 | UCUCAUUUCUCU | 1017 | UGUCAGAGAAAUGAG |
| | UUCUCUGACC | | GACA | | AGGGAA |
| 250 | CCUCUCAUUUC | 634 | CAUUUCUCUGAC | 1018 | UUCGGUCAGAGAAAU |
| | UCUGACCGAA | | CGAA | | GAGAGG |
| 251 | CUCUCAUUUCU | 635 | AUUUCUCUGACC | 1019 | UUUCGGUCAGAGAAA |
| | CUGACCGAAG | | GAAA | | UGAGAG |
| 252 | UUCUCUGACCG | 636 | UGACCGAAGCUG | 1020 | UACCCAGCUUCGGUC |
| | AAGCUGGGUG | | GGUA | | AGAGAA |
| 253 | CUUUCGCCUCU | 637 | GCCUCUAGCGAC | 1021 | UCCAGUCGCUAGAGG |
| | AGCGACUGGU | | UGGA | | CGAAAG |
| 254 | UUUCGCCUCUA | 638 | CCUCUAGCGACU | 1022 | UACCAGUCGCUAGAG |
| | GCGACUGGUG | | GGUA | | GCGAAA |
| 255 | UCGCCUCUAGC | 639 | UCUAGCGACUGG | 1023 | UCCACCAGUCGCUAG |
| | GACUGGUGGA | | UGGA | | AGGCGA |
| 256 | CGCCUCUAGCG | 640 | CUAGCGACUGGU | 1024 | UUCCACCAGUCGCUA |
| | ACUGGUGGAA | | GGAA | | GAGGCG |
| 257 | UUCCCGCCCUC | 641 | GCCCUCAGUACC | 1025 | UUCGGGUACUGAGG |
| | AGUACCCGAG | | CGAA | | GCGGGAA |
| 258 | GGAGACGCCUG | 642 | CGCCUGCACAAU | 1026 | UGAAAUUGUGCAGGC |
| | CACAAUUUCA | | UUCA | | GUCUCC |
| 259 | CGCCUGCACAA | 643 | GCACAAUUUCAG | 1027 | UGGGCUGAAAUUGUG |
| | UUUCAGCCCA | | CCCA | | CAGGCG |
| 260 | UCUAGAGAGUG | 644 | AGAGUGGUGAUG | 1028 | UAGUCAUCACCACUC |
| | GUGAUGACUU | | ACUA | | UCUAGA |
| 261 | UAGAGAGUGGU | 645 | AGUGGUGAUGAC | 1029 | UCAAGUCAUCACCAC |
| | GAUGACUUGC | | UUGA | | UCUCUA |
| 262 | GGUGAUGACUU | 646 | UGACUUGCAUAU | 1030 | UCUCAUAUGCAAGUC |
| | GCAUAUGAGG | | GAGA | | AUCACC |
| 263 | UGAUGACUUGC | 647 | ACUUGCAUAUGA | 1031 | UCCCUCAUAUGCAAG |
| | AUAUGAGGGC | | GGGA | | UCAUCA |
| 264 | GAUGACUUGCA | 648 | CUUGCAUAUGAG | 1032 | UGCCCUCAUAUGCAA |
| | UAUGAGGGCA | | GGCA | | GUCAUC |
| 265 | CUUGCAUAUGA | 649 | AUAUGAGGGCAG | 1033 | UUUGCUGCCCUCAUA |
| | GGGCAGCAAU | | CAAA | | UGCAAG |
| 266 | AUAUGAGGGCA | 650 | AGGGCAGCAAUG | 1034 | UUUGCAUUGCUGCCC |
| | GCAAUGCAAG | | CAAA | | UCAUAU |
| 267 | UGAGGGCAGCA | 651 | GCAGCAAUGCAA | 1035 | UGACUUGCAUUGCUG |
| | AUGCAAGUCG | | GUCA | | CCCUCA |
| 268 | GGCAGCAAUGC | 652 | CAAUGCAAGUCG | 1036 | UCACCGACUUGCAUU |
| | AAGUCGGUGU | | GUGA | | GCUGCC |
| 269 | GUGGGACAUGA | 653 | ACAUGACCUGGU | 1037 | UGCAACCAGGUCAUG |
| | CCUGGUUGCU | | UGCA | | UCCCAC |
| 270 | CCUGGUUGCUU | 654 | UUGCUUCACAGC | 1038 | UGGAGCUGUGAAGCA |
| | CACAGCUCCG | | UCCA | | ACCAGG |
| 271 | UGGUUGCUUCA | 655 | GCUUCACAGCUC | 1039 | UUCGGAGCUGUGAAG |
| | CAGCUCCGAG | | CGAA | | CAACCA |
| 272 | UUGCUUCACAG | 656 | UCACAGCUCCGA | 1040 | UAUCUCGGAGCUGUG |
| | CUCCGAGAUG | | GAUA | | AAGCAA |
| 273 | UCACAGCUCCG | 657 | GCUCCGAGAUGA | 1041 | UGUGUCAUCUCGGAG |
| | AGAUGACACA | | CACA | | CUGUGA |
| 274 | CUCCGAGAUGA | 658 | AGAUGACACAGAC | 1042 | UAAGUCUGUGUCAUC |
| | CACAGACUUG | | UUA | | UCGGAG |
| 275 | UCCGAGAUGAC | 659 | GAUGACACAGAC | 1043 | UCAAGUCUGUGUCAU |
| | ACAGACUUGC | | UUGA | | CUCGGA |
| 276 | CCGAGAUGACA | 660 | AUGACACAGACUU | 1044 | UGCAAGUCUGUGUCA |
| | CAGACUUGCU | | GCA | | UCUCGG |
| 277 | AGAUGACACAGA | 661 | ACACAGACUUGC | 1045 | UUAAGCAAGUCUGUG |
| | CUUGCUUAA | | UUAA | | UCAUCU |
| 278 | AUGACACAGACU | 662 | ACAGACUUGCUU | 1046 | UUUUAAGCAAGUCUG |
| | UGCUUAAAG | | AAAA | | UGUCAU |
| 279 | CACAGACUUGC | 663 | ACUUGCUUAAAG | 1047 | UUUCCUUUAAGCAAG |
| | UUAAAGGAAG | | GAAA | | UCUGUG |
| 280 | CUUGCUUAAAG | 664 | UUAAAGGAAGUG | 1048 | UAGUCACUUCCUUUA |
| | GAAGUGACUA | | ACUA | | AGCAAG |
| 281 | UGCUUAAAGGA | 665 | AAAGGAAGUGAC | 1049 | UAUAGUCACUUCCUU |
| | AGUGACUAUU | | UAUA | | UAAGCA |
| 282 | UUAAAGGAAGU | 666 | GGAAGUGACUAU | 1050 | UACAAUAGUCACUUC |
| | GACUAUUGUG | | UGUA | | CUUUAA |
| 283 | UAAAGGAAGUG | 667 | GAAGUGACUAUU | 1051 | UCACAAUAGUCACUU |
| | ACUAUUGUGA | | GUGA | | CCUUUA |
| 284 | GAAGUGACUAU | 668 | GACUAUUGUGAC | 1052 | UCAAGUCACAAUAGU |
| | UGUGACUUGG | | UUGA | | CACUUC |
| 285 | GUGACUAUUGU | 669 | UAUUGUGACUUG | 1053 | UGCCCAAGUCACAAU |
| | GACUUGGGCA | | GGCA | | AGUCAC |
| 286 | GACUAUUGUGA | 670 | UUGUGACUUGGG | 1054 | UAUGCCCAAGUCACA |
| | CUUGGGCAUC | | CAUA | | AUAGUC |
| 287 | UUGUGACUUGG | 671 | ACUUGGGCAUCA | 1055 | UAAGUGAUGCCCAAG |
| | GCAUCACUUG | | CUUA | | UCACAA |
| 288 | UGUGACUUGGG | 672 | CUUGGGCAUCAC | 1056 | UCAAGUGAUGCCCAA |
| | CAUCACUUGA | | UUGA | | GUCACA |
| 289 | GUGACUUGGGC | 673 | UUGGGCAUCACU | 1057 | UUCAAGUGAUGCCCA |
| | AUCACUUGAC | | UGAA | | AGUCAC |
| 290 | UGACUUGGGCA | 674 | UGGGCAUCACUU | 1058 | UGUCAAGUGAUGCCC |
| | UCACUUGACU | | GACA | | AAGUCA |
| 291 | CUUGGGCAUCA | 675 | GCAUCACUUGAC | 1059 | UUCAGUCAAGUGAUG |
| | CUUGACUGAU | | UGAA | | CCCAAG |
| 292 | UUGGGCAUCAC | 676 | CAUCACUUGACU | 1060 | UAUCAGUCAAGUGAU |
| | UUGACUGAUG | | GAUA | | GCCCAA |
| 293 | UCACUUGACUG | 677 | UGACUGAUGGUA | 1061 | UGAUUACCAUCAGUC |
| | AUGGUAAUCA | | AUCA | | AAGUGA |
| 294 | ACUUGACUGAU | 678 | ACUGAUGGUAAU | 1062 | UCUGAUUACCAUCAG |
| | GGUAAUCAGU | | CAGA | | UCAAGU |
| 295 | UUGACUGAUGG | 679 | UGAUGGUAAUCA | 1063 | UAACUGAUUACCAUC |
| | UAAUCAGUUG | | GUUA | | AGUCAA |
| 296 | UGACUGAUGGU | 680 | GAUGGUAAUCAG | 1064 | UCAACUGAUUACCAU |
| | AAUCAGUUGU | | UUGA | | CAGUCA |
| 297 | GACUGAUGGUA | 681 | AUGGUAAUCAGU | 1065 | UACAACUGAUUACCA |
| | AUCAGUUGUC | | UGUA | | UCAGUC |
| 298 | UGAUGGUAAUC | 682 | GUAAUCAGUUGU | 1066 | UUAGACAACUGAUUA |
| | AGUUGUCUAA | | CUAA | | CCAUCA |
| 299 | GAUGGUAAUCA | 683 | UAAUCAGUUGUC | 1067 | UUUAGACAACUGAUU |
| | GUUGUCUAAA | | UAAA | | ACCAUC |
| 300 | GGUAAUCAGUU | 684 | UCAGUUGUCUAA | 1068 | UUCUUUAGACAACUG |
| | GUCUAAAGAA | | AGAA | | AUUACC |
| 301 | AUCAGUUGUCU | 685 | UUGUCUAAAGAA | 1069 | UCACUUCUUUAGACA |
| | AAAGAAGUGC | | GUGA | | ACUGAU |
| 302 | UCAGUUGUCUA | 686 | UGUCUAAAGAAG | 1070 | UGCACUUCUUUAGAC |
| | AAGAAGUGCA | | UGCA | | AACUGA |
| 303 | GUUGUCUAAAG | 687 | CUAAAGAAGUGCA | 1071 | UUGUGCACUUCUUUA |
| | AAGUGCACAG | | CAA | | GACAAC |
| 304 | UUGUCUAAAGAA | 688 | UAAAGAAGUGCAC | 1072 | UCUGUGCACUUCUUU |
| | GUGCACAGA | | AGA | | AGACAA |
| 305 | UGUCUAAAGAA | 689 | AAAGAAGUGCACA | 1073 | UUCUGUGCACUUCUU |
| | GUGCACAGAU | | GAA | | UAGACA |
| 306 | GUCUAAAGAAG | 690 | AAGAAGUGCACA | 1074 | UAUCUGUGCACUUCU |
| | UGCACAGAUU | | GAUA | | UUAGAC |
| 307 | UCUAAAGAAGU | 691 | AGAAGUGCACAG | 1075 | UAAUCUGUGCACUUC |
| | GCACAGAUUA | | AUUA | | UUUAGA |
| 308 | CUAAAGAAGUG | 692 | GAAGUGCACAGA | 1076 | UUAAUCUGUGCACUU |
| | CACAGAUUAC | | UUAA | | CUUUAG |
| 309 | UAAAGAAGUGCA | 693 | AAGUGCACAGAU | 1077 | UGUAAUCUGUGCACU |
| | CAGAUUACA | | UACA | | UCUUUA |
| 310 | AGUGCACAGAU | 694 | ACAGAUUACAUGU | 1078 | UGGACAUGUAAUCUG |
| | UACAUGUCCG | | CCA | | UGCACU |
| 311 | UGCACAGAUUA | 695 | AGAUUACAUGUC | 1079 | UACGGACAUGUAAUC |
| | CAUGUCCGUG | | CGUA | | UGUGCA |
| 312 | AGAUUACAUGU | 696 | ACAUGUCCGUGU | 1080 | UAGCACACGGACAUG |
| | CCGUGUGCUC | | GCUA | | UAAUCU |
| 313 | CGUGUGCUCAU | 697 | GCUCAUUGGGUC | 1081 | UAUAGACCCAAUGAG |
| | UGGGUCUAUC | | UAUA | | CACACG |
| 314 | UGUGCUCAUUG | 698 | UCAUUGGGUCUA | 1082 | UAGAUAGACCCAAUG |
| | GGUCUAUCUG | | UCUA | | AGCACA |
| 315 | UCAUUGGGUCU | 699 | GGGUCUAUCUGG | 1083 | UCGGCCAGAUAGACC |
| | AUCUGGCCGC | | CCGA | | CAAUGA |
| 316 | GUCUAUCUGGC | 700 | UCUGGCCGCGUU | 1084 | UUUCAACGCGGCCAG |
| | CGCGUUGAAC | | GAAA | | AUAGAC |
| 317 | CCGCGUUGAAC | 701 | UUGAACACCACCA | 1085 | UCCUGGUGGUGUUCA |
| | ACCACCAGGC | | GGA | | ACGCGG |
| 318 | GUUGAACACCA | 702 | ACACCACCAGGC | 1086 | UAAAGCCUGGUGGUG |
| | CCAGGCUUUG | | UUUA | | UUCAAC |
| 319 | UGAACACCACCA | 703 | ACCACCAGGCUU | 1087 | UACAAAGCCUGGUGG |
| | GGCUUUGUA | | UGUA | | UGUUCA |
| 320 | GAACACCACCAG | 704 | CCACCAGGCUUU | 1088 | UUACAAAGCCUGGUG |
| | GCUUUGUAU | | GUAA | | GUGUUC |
| 321 | UUUGUAUUCAG | 705 | AUUCAGAAACAGG | 1089 | UCUCCUGUUUCUGAA |
| | AAACAGGAGG | | AGA | | UACAAA |
| 322 | CAGAAACAGGA | 706 | ACAGGAGGGAGG | 1090 | UGGACCUCCCUCCUG |
| | GGGAGGUCCU | | UCCA | | UUUCUG |
| 323 | GAGGGAGGUCC | 707 | AGGUCCUGCACU | 1091 | UGAAAGUGCAGGACC |
| | UGCACUUUCC | | UUCA | | UCCCUC |
| 324 | GGUGGCCCUUU | 708 | CCCUUUCAGAUG | 1092 | UUUGCAUCUGAAAGG |
| | CAGAUGCAAU | | CAAA | | GCCACC |
| 325 | GUGGCCCUUUC | 709 | CCUUUCAGAUGC | 1093 | UAUUGCAUCUGAAAG |
| | AGAUGCAAUC | | AAUA | | GGCCAC |
| 326 | UGGCCCUUUCA | 710 | CUUUCAGAUGCA | 1094 | UGAUUGCAUCUGAAA |
| | GAUGCAAUCG | | AUCA | | GGGCCA |
| 327 | CCCUUUCAGAU | 711 | UCAGAUGCAAUC | 1095 | UCUCGAUUGCAUCUG |
| | GCAAUCGAGA | | GAGA | | AAAGGG |
| 328 | UUUCAGAUGCA | 712 | GAUGCAAUCGAG | 1096 | UAAUCUCGAUUGCAU |
| | AUCGAGAUUG | | AUUA | | CUGAAA |
| 329 | CAGAUGCAAUC | 713 | GCAAUCGAGAUU | 1097 | UAACAAUCUCGAUUG |
| | GAGAUUGUUA | | GUUA | | CAUCUG |
| 330 | AAUCGAGAUUG | 714 | AGAUUGUUAGGC | 1098 | UAGAGCCUAACAAUC |
| | UUAGGCUCUG | | UCUA | | UCGAUU |
| 331 | AUCGAGAUUGU | 715 | GAUUGUUAGGCU | 1099 | UCAGAGCCUAACAAU |
| | UAGGCUCUGG | | CUGA | | CUCGAU |
| 332 | UCGAGAUUGUU | 716 | AUUGUUAGGCUC | 1100 | UCCAGAGCCUAACAA |
| | AGGCUCUGGG | | UGGA | | UCUCGA |
| 333 | CGAGAUUGUUA | 717 | UUGUUAGGCUCU | 1101 | UCCCAGAGCCUAACA |
| | GGCUCUGGGA | | GGGA | | AUCUCG |
| 334 | GAGAUUGUUAG | 718 | UGUUAGGCUCUG | 1102 | UUCCCAGAGCCUAAC |
| | GCUCUGGGAG | | GGAA | | AAUCUC |
| 335 | UUGUUAGGCUC | 719 | AGGCUCUGGGAG | 1103 | UACUCUCCCAGAGCC |
| | UGGGAGAGUA | | AGUA | | UAACAA |
| 336 | GUUAGGCUCUG | 720 | GCUCUGGGAGAG | 1104 | UCUACUCUCCCAGAG |
| | GGAGAGUAGU | | UAGA | | CCUAAC |
| 337 | UUAGGCUCUGG | 721 | CUCUGGGAGAGU | 1105 | UACUACUCUCCCAGA |
| | GAGAGUAGUU | | AGUA | | GCCUAA |
| 338 | UAGGCUCUGGG | 722 | UCUGGGAGAGUA | 1106 | UAACUACUCUCCCAG |
| | AGAGUAGUUG | | GUUA | | AGCCUA |
| 339 | GGCUCUGGGAG | 723 | UGGGAGAGUAGU | 1107 | UGCAACUACUCUCCC |
| | AGUAGUUGCC | | UGCA | | AGAGCC |
| 340 | CUGGGAGAGUA | 724 | AGAGUAGUUGCC | 1108 | UCCAGGCAACUACUC |
| | GUUGCCUGGU | | UGGA | | UCCCAG |
| 341 | GGGAGAGUAGU | 725 | AGUAGUUGCCUG | 1109 | UAACCAGGCAACUAC |
| | UGCCUGGUUG | | GUUA | | UCUCCC |
| 342 | GAGAGUAGUUG | 726 | UAGUUGCCUGGU | 1110 | UACAACCAGGCAACU |
| | CCUGGUUGUG | | UGUA | | ACUCUC |
| 343 | UUGCCUGGUUG | 727 | UGGUUGUGGCAG | 1111 | UCAACUGCCACAACC |
| | UGGCAGUUGG | | UUGA | | AGGCAA |
| 344 | UGCCUGGUUGU | 728 | GGUUGUGGCAGU | 1112 | UCCAACUGCCACAAC |
| | GGCAGUUGGU | | UGGA | | CAGGCA |
| 345 | GCCUGGUUGUG | 729 | GUUGUGGCAGUU | 1113 | UACCAACUGCCACAA |
| | GCAGUUGGUA | | GGUA | | CCAGGC |
| 346 | CCUGGUUGUGG | 730 | UUGUGGCAGUUG | 1114 | UUACCAACUGCCACA |
| | CAGUUGGUAA | | GUAA | | ACCAGG |
| 347 | CUGGUUGUGGC | 731 | UGUGGCAGUUGG | 1115 | UUUACCAACUGCCAC |
| | AGUUGGUAAA | | UAAA | | AACCAG |
| 348 | UGGUUGUGGCA | 732 | GUGGCAGUUGGU | 1116 | UUUUACCAACUGCCA |
| | GUUGGUAAAU | | AAAA | | CAACCA |
| 349 | UUGUGGCAGUU | 733 | GCAGUUGGUAAA | 1117 | UAAAUUUACCAACUG |
| | GGUAAAUUUC | | UUUA | | CCACAA |
| 350 | UGUGGCAGUUG | 734 | CAGUUGGUAAAU | 1118 | UGAAAUUUACCAACU |
| | GUAAAUUUCU | | UUCA | | GCCACA |
| 351 | GUGGCAGUUGG | 735 | AGUUGGUAAAUU | 1119 | UAGAAAUUUACCAAC |
| | UAAAUUUCUA | | UCUA | | UGCCAC |
| 352 | UGGCAGUUGGU | 736 | GUUGGUAAAUUU | 1120 | UUAGAAAUUUACCAA |
| | AAAUUUCUAU | | CUAA | | CUGCCA |
| 353 | GGCAGUUGGUA | 737 | UUGGUAAAUUUC | 1121 | UAUAGAAAUUUACCA |
| | AAUUUCUAUU | | UAUA | | ACUGCC |
| 354 | GCAGUUGGUAA | 738 | UGGUAAAUUUCU | 1122 | UAAUAGAAAUUUACC |
| | AUUUCUAUUC | | AUUA | | AACUGC |
| 355 | CAGUUGGUAAA | 739 | GGUAAAUUUCUA | 1123 | UGAAUAGAAAUUUAC |
| | UUUCUAUUCA | | UUCA | | CAACUG |
| 356 | UCUAUUCAAACA | 740 | UCAAACAGUUGC | 1124 | UAUGGCAACUGUUUG |
| | GUUGCCAUG | | CAUA | | AAUAGA |
| 357 | UAUUCAAACAGU | 741 | AAACAGUUGCCAU | 1125 | UGCAUGGCAACUGUU |
| | UGCCAUGCA | | GCA | | UGAAUA |
| 358 | AUUCAAACAGUU | 742 | AACAGUUGCCAU | 1126 | UUGCAUGGCAACUGU |
| | GCCAUGCAC | | GCAA | | UUGAAU |
| 359 | UUCAAACAGUU | 743 | ACAGUUGCCAUG | 1127 | UGUGCAUGGCAACUG |
| | GCCAUGCACC | | CACA | | UUUGAA |
| 360 | CAGUUGCCAUG | 744 | GCCAUGCACCAG | 1128 | UCAACUGGUGCAUGG |
| | CACCAGUUGU | | UUGA | | CAACUG |
| 361 | AGUUGCCAUGC | 745 | CCAUGCACCAGU | 1129 | UACAACUGGUGCAUG |
| | ACCAGUUGUU | | UGUA | | GCAACU |
| 362 | UUGCCAUGCAC | 746 | AUGCACCAGUUG | 1130 | UGAACAACUGGUGCA |
| | CAGUUGUUCA | | UUCA | | UGGCAA |
| 363 | UGCCAUGCACC | 747 | UGCACCAGUUGU | 1131 | UUGAACAACUGGUGC |
| | AGUUGUUCAC | | UCAA | | AUGGCA |
| 364 | GCCAUGCACCA | 748 | GCACCAGUUGUU | 1132 | UGUGAACAACUGGUG |
| | GUUGUUCACA | | CACA | | CAUGGC |
| 365 | AUGCACCAGUU | 749 | CCAGUUGUUCAC | 1133 | UGUUGUGAACAACUG |
| | GUUCACAACA | | AACA | | GUGCAU |
| 366 | GUUGUUCACAA | 750 | UCACAACAAGGG | 1134 | UGUACCCUUGUUGUG |
| | CAAGGGUACG | | UACA | | AACAAC |
| 367 | UUCACAACAAGG | 751 | AACAAGGGUACG | 1135 | UUUACGUACCCUUGU |
| | GUACGUAAU | | UAAA | | UGUGAA |
| 368 | UCACAACAAGG | 752 | ACAAGGGUACGU | 1136 | UAUUACGUACCCUUG |
| | GUACGUAAUC | | AAUA | | UUGUGA |
| 369 | CACAACAAGGG | 753 | CAAGGGUACGUA | 1137 | UGAUUACGUACCCUU |
| | UACGUAAUCU | | AUCA | | GUUGUG |
| 370 | CAAGGGUACGU | 754 | GUACGUAAUCUG | 1138 | UAGACAGAUUACGUA |
| | AAUCUGUCUG | | UCUA | | CCCUUG |
| 371 | AGGGUACGUAA | 755 | ACGUAAUCUGUC | 1139 | UCCAGACAGAUUACG |
| | UCUGUCUGGC | | UGGA | | UACCCU |
| 372 | GUACGUAAUCU | 756 | UAAUCUGUCUGG | 1140 | UAUGCCAGACAGAUU |
| | GUCUGGCAUU | | CAUA | | ACGUAC |
| 373 | UACGUAAUCUG | 757 | AAUCUGUCUGGC | 1141 | UAAUGCCAGACAGAU |
| | UCUGGCAUUA | | AUUA | | UACGUA |
| 374 | CGUAAUCUGUC | 758 | UCUGUCUGGCAU | 1142 | UGUAAUGCCAGACAG |
| | UGGCAUUACU | | UACA | | AUUACG |
| 375 | AUCUGUCUGGC | 759 | UCUGGCAUUACU | 1143 | UAGAAGUAAUGCCAG |
| | AUUACUUCUA | | UCUA | | ACAGAU |
| 376 | CUGUCUGGCAU | 760 | UGGCAUUACUUC | 1144 | UGUAGAAGUAAUGCC |
| | UACUUCUACU | | UACA | | AGACAG |
| 377 | UCUGGCAUUAC | 761 | CAUUACUUCUACU | 1145 | UAAAGUAGAAGUAAU |
| | UUCUACUUUU | | UUA | | GCCAGA |
| 378 | CUGGCAUUACU | 762 | AUUACUUCUACUU | 1146 | UAAAAGUAGAAGUAA |
| | UCUACUUUUG | | UUA | | UGCCAG |
| 379 | UGGCAUUACUU | 763 | UUACUUCUACUU | 1147 | UCAAAAGUAGAAGUA |
| | CUACUUUUGU | | UUGA | | AUGCCA |
| 380 | UCUACUUUUGU | 764 | UUUUGUACAAAG | 1148 | UAUCCUUUGUACAAA |
| | ACAAAGGAUC | | GAUA | | AGUAGA |
| 381 | CUACUUUUGUA | 765 | UUUGUACAAAGG | 1149 | UGAUCCUUUGUACAA |
| | CAAAGGAUCA | | AUCA | | AAGUAG |
| 382 | UACUUUUGUAC | 766 | UUGUACAAAGGA | 1150 | UUGAUCCUUUGUACA |
| | AAAGGAUCAA | | UCAA | | AAAGUA |
| 383 | ACUUUUGUACAA | 767 | UGUACAAAGGAU | 1151 | UUUGAUCCUUUGUAC |
| | AGGAUCAAA | | CAAA | | AAAAGU |
| 384 | CUUUUGUACAAA | 768 | GUACAAAGGAUCA | 1152 | UUUUGAUCCUUUGUA |
| | GGAUCAAAA | | AAA | | CAAAAG |

siRNA Structure

The siRNA molecules of the disclosure may be in the form of a single-stranded (ss) or double-stranded (ds) oligonucleotide structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNAi activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, preferably an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

The siRNA molecules described herein may employ modifications to the nucleobase, phosphate backbone, ribose core, 5′- and 3′-ends, and branching, wherein multiple strands of siRNA may be covalently linked.

### Lengths of Small Interfering RNA Molecules

It is within the scope of the disclosure that any length, known and previously unknown in the art, may be employed for the current invention. As described herein, potential lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the siRNA molecules of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 2′ Sugar Modifications

The present disclosure may include ss- and ds-siRNA molecule compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O—, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cc, Br, CN, CF₃, OCF₃, SOCH₃, SO₂CH₃, ONO, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### Nucleobase Modifications

The siRNA molecules of the disclosure may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. The siRNA molecules of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### Internucleoside Linkage Modifications

Another variable in the design of the present disclosure is the internucleoside linkage making up the phosphate backbone of the siRNA molecule. Although the natural RNA phosphate backbone may be employed here, derivatives thereof may be used which enhance desirable characteristics of the siRNA molecule. Although not limiting, of particular importance in the present disclosure is protecting parts, or the whole, of the siRNA molecule from hydrolysis. One example of a modification that decreases the rate of hydrolysis is phosphorothioates. Any portion or the whole of the backbone may contain phosphate substitutions (e.g., phosphorothioates). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential siRNA molecules useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. A preferred phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Patterns of Modifications of siRNA Molecules

The following section provides a set of exemplary scaffolds into which the siRNA molecules of the disclosure may be incorporated.

In some embodiments of the disclosure, the siRNA may contain an antisense strand including a region represented by Formula I, wherein Formula I is, in the 5′-to-3′ direction

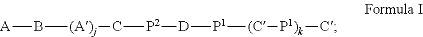

wherein A is represented by the formula C—P¹-D-P¹; each A′ is represented by the formula C—P²-D-P²; B is represented by the formula C—P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

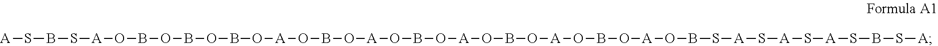

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′-to-3′ direction:

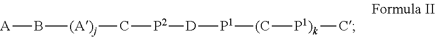

wherein A is represented by the formula C—P¹-D-P¹; each A′ is represented by the formula C—P²-D-P²; B is represented by the formula C—P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

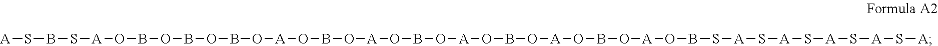

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′-to-3′ direction:

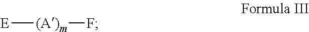

wherein E is represented by the formula (C—P¹)₂; F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

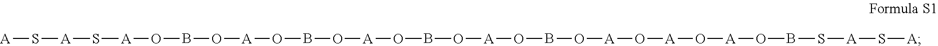

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′-to-3′ direction:

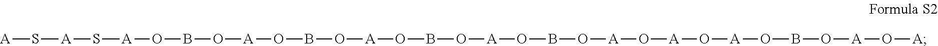

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

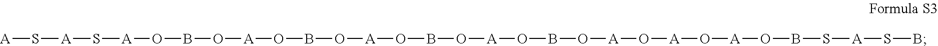

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

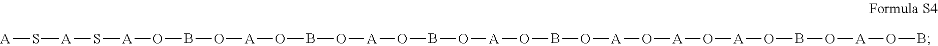

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′-to-3′ direction:

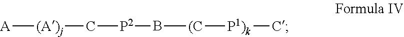

wherein A is represented by the formula C—P¹-D-P¹; each A′ is represented by the formula C—P²-D-P²; B is represented by the formula D-P¹—C—P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′-to-3′ direction:

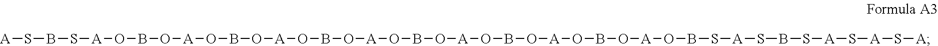

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′-to-3′ direction:

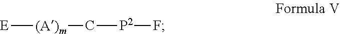

wherein E is represented by the formula (C—P¹)₂; F is represented by the formula D-P¹—C—P¹—C, D-P²—C—P²—C, D-P¹—C—P¹-D, or D-P²—C—P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

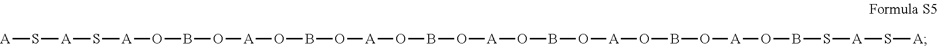

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

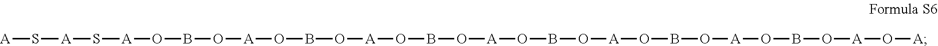

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S7, wherein Formula S7 is, in the 5′-to-3′ direction:

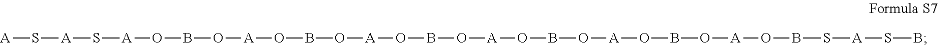

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

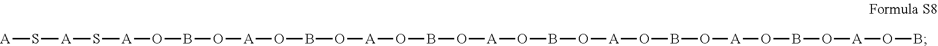

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′-to-3′ direction:

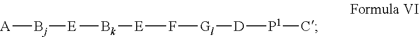

wherein A is represented by the formula C—P¹-D-P¹; each B is represented by the formula C—P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²—C—P²; F is represented by the formula D-P¹—C—P¹; each G is represented by the formula C—P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and l is an integer from 1 to 7 (e.g., 1, 2, 3, 4, 5, 6, or 7). In some embodiments, j is 3. In some embodiments, k is 6. In some embodiments, l is 2. In some embodiments, j is 3, k is 6, and l is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

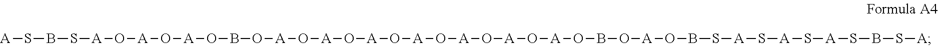

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′-to-3′ direction:

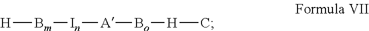

wherein A′ is represented by the formula C—P²-D-P²; each H is represented by the formula (C—P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

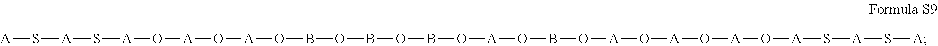

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

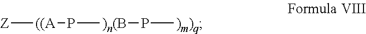

wherein Z is a 5′ phosphorus stabilizing moiety; each A is a 2′-O-methyl (2′-O-Me) ribonucleoside; each B is a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).
   Methods of siRNA Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

### 5′ Phosphorus Stabilizing Moieties

To further protect the siRNA molecules of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each strand of a siRNA molecule may independently and optionally employ any suitable 5′-phosphorus stabilizing moiety.

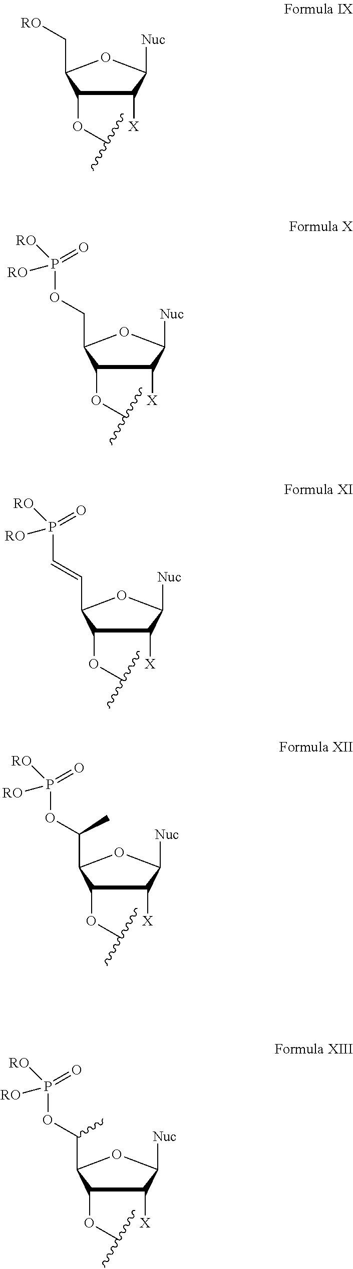

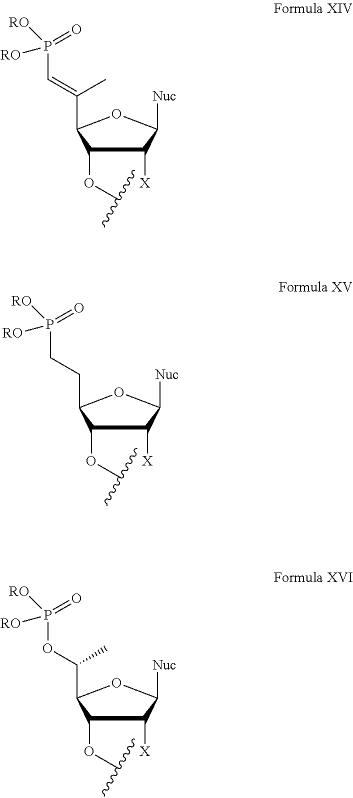

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula IX, phosphate as in Formula X, vinylphosphonates as in Formula XI and XIV, 5′-methyl-substituted phosphates as in Formula XII, XIII, and XVI, methylenephosphonates as in Formula XV, or vinyl 5′-vinylphsophonate as a 5′-phosphorus stabilizing moiety as demonstrated in Formula XI.

### Hydrophobic Moieties

The present disclosure further provides siRNA molecules having one or more hydrophobic moieties attached thereto. The hydrophobic moiety may be covalently attached to the 5′ end or the 3′ end of the siRNA molecules of the disclosure. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

siRNA Branching

The siRNA molecules of the disclosure may be branched. For example, the siRNA molecules of the disclosure may have one of several branching patterns, as described herein.

According to the present disclosure, the siRNA molecules disclosed herein may be branched SiRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2.

TABLE 2
Branched siRNA structures

| Di-branched | Tri-branched | Tetra-branched |
|---|---|---|
| RNA-L-RNA Formula XVII | 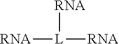 | 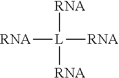 |
| | Formula XX | Formula XXIV |
| 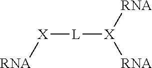 | 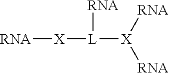 | 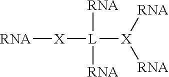 |
| Formula XVIII | Formula XXI | Formula XXV |
| 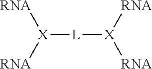 | 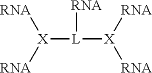 | 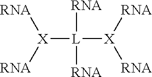 |
| Formula XIX | Formula XXII | Formula XXVI |
| | 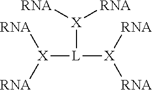 | 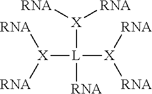 |
| | Formula XXIII | Formula XXVII |
| | | 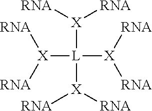 |
| | | Formula XXVIII |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVII-XIX, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XX-XXIII, wherein each RNA, independently, is an siRNA molecule, L is a linker, and each X, independently, represents a branch point moiety.

In some embodiments, the tetra-branched siRNA molecule represented by any one of Formulas XXIV-XXVIII, wherein each RNA, independently, is an siRNA molecule, L is a linker, and each X, independently, represents a branch point moiety.

### Linkers

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene glycol (TEG) linker.

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.).

In some embodiments, the linker has a structure of Formula L1:

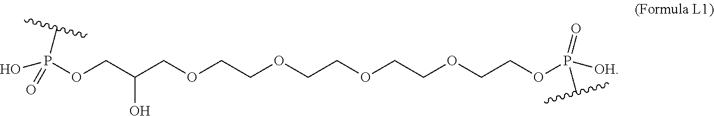 [SMILES: *P(=O)(O)OCCOCCOCCOCCOCC(O)COP(*)(=O)O]

In some embodiments, the linker has a structure of Formula L2:

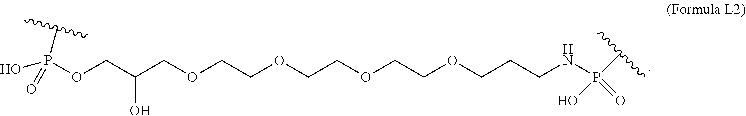 [SMILES: *P(=O)(O)NCCCOCCOCCOCCOCC(O)COP(*)(=O)O]

In some embodiments, the linker has a structure of Formula L3:

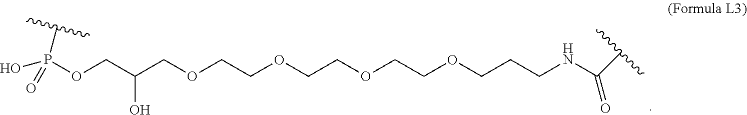 [SMILES: *C(=O)NCCCOCCOCCOCCOCC(O)COP(*)(=O)O]

In some embodiments, the linker has a structure of Formula L4:

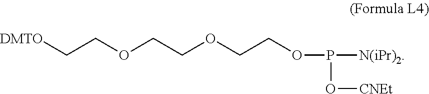

In some embodiments, the linker has a structure of Formula L5:

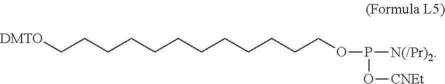

In some embodiments, the linker has a structure of Formula L6:

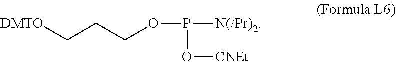

In some embodiments, the linker has a structure of Formula L7, as is shown below:

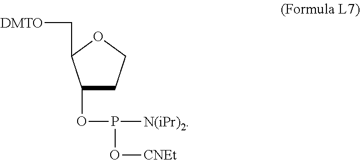

In some embodiments, the linker has a structure of Formula L8:

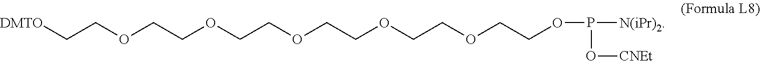

In some embodiments, the linker has a structure of Formula L9:

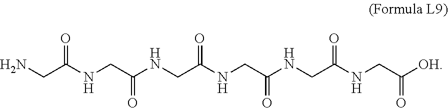 [SMILES: NCC(=O)NCC(=O)NCC(=O)NCC(=O)NCC(=O)NCC(=O)O]

In some embodiments, the selection of a linker for use with one or more of the branched SIRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### Methods of Treatment

The C9orf72-targeting siRNA molecules of the disclosure may be delivered to a subject, thereby treating amyotrophic lateral sclerosis (ALS), frontotemporal dementia (FTD), and/or mitigating phenotypes associated with the disorders. For example, the siRNA molecules may be delivered to a subject, thereby treating ALS and/or mitigating ALS disease associated phenotypes (e.g., involuntary movements, muscle cramps, weakness, loss of motor control). Alternatively, the C9orf72-targeting siRNA molecules of the disclosure may be delivered to a subject, thereby treating FTD and/or mitigating FTD-associated phenotypes (e.g., memory problems, behavioral problems, and language problems). Furthermore, the siRNA molecules of the disclosure may also be delivered to a subject having a variant of the C9orf72 gene for which siRNA-mediated gene silencing of the C9orf72 variant gene reduces the expression level of C9orf72transcript, thereby treating ALS, FTD, or other C9orf72-related diseases or disorders.

The disclosure provides methods of treating a subject by way of C9orf72 gene silencing with one or more of the siRNA molecules described herein. The gene silencing may be performed in a subject to silence wild type C9orf72 transcripts, mutant C9orf72 transcripts, splice isoforms of C9orf72 transcripts, and/or overexpressed C9orf72 transcripts thereof, relative to a healthy subject. The method may include delivering to the CNS or affected tissues of the subject (e.g., a human) the SiRNA molecules of the disclosure or a pharmaceutical composition containing the same by any appropriate route of administration (e.g., intracerebroventricular, intrathecal injection, intrastriatal injection, intra-cisterna magna injection by catheterization, intraparenchymal injection, intravenous injection, subcutaneous injection, or intramuscular injection). The active compound can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### Selection of Subjects

Subjects that may be treated with the siRNA molecules disclosed herein are subjects in need of treatment of, for example, ALS, FTD, and/or any other medical risk(s) associated with repeat expansions or a gain of function mutations in the C9orf72 gene. Subjects that may be treated with the siRNA molecules disclosed herein may include, for example, humans, monkeys, rats, mice, pigs, and other mammals containing at least one orthologous copy of the C9orf72 gene. Subjects may be adult or pediatric humans, with or without comorbid diseases.

### Pharmaceutical Compositions

The siRNA molecules in the present disclosure may be formulated into a pharmaceutical composition for administration to a subject in a biologically compatible form suitable for administration in vivo. Accordingly, the present disclosure provides a pharmaceutical composition containing a siRNA molecule of the disclosure in admixture with a suitable diluent, carrier, or excipient. The siRNA molecules may be administered, for example, directly into the CNS or affected tissues of the subject (e.g., by way of intracerebroventricular, intrastriatally, intrathecal injection, intra-cisterna magna injection by catheterization, intraparenchymal injection, intravenous injection, subcutaneous injection, or intramuscular injection).

Conventional procedures and ingredients for the selection and preparation of suitable formulations are described, for example, in Remington, J. P. The Science and Practice of Pharmacy, Easton, PA. Mack Publishers, 2012, 22nd ed. and in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### Dosing Regimens

A physician having ordinary skill in the art can readily determine an effective amount of the siRNA molecule for administration to a mammalian subject (e.g., a human) in need thereof. For example, a physician could start prescribing doses of one the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until reaching a minimal dosage at which a therapeutic effect is achieved (e.g., a reduction in expression of a target gene sequence). In general, a suitable daily dose of one of the siRNA molecules of the disclosure will be an amount of the siRNA molecule which is the lowest dose effective to produce a therapeutic effect. The ss- or ds-siRNA molecules of the disclosure may be administered by injection, e.g., intrathecally, intracerebroventricularly, by intra-cisterna magna injection by catheterization, intraparenchymally, intravenously, subcutaneously, or intramuscularly. A daily dose of a therapeutic composition of the siRNA molecules of the disclosure may be administered as a single dose or as two, three, four, five, six or more doses administered separately at appropriate intervals throughout the day, week, month, or year, optionally, in unit dosage forms. While it is possible for the siRNA molecules of the disclosure to be administered alone, it may also be administered as a pharmaceutical formulation in combination with excipients, carriers, and optionally, additional therapeutic agents.

### Routes of Administration

The method of the disclosure contemplates any route of administration tolerated by the therapeutic composition. Some embodiments of the method include injection intrathecally, intracerebroventricularly, intrastriatally, intraparenchymally, or by intra-cisterna magna injection by catheterization.

Intrathecal injection is the direct injection into the spinal column or subarachnoid space. By injecting directly into the CSF of the spinal column the siRNA molecules of the disclosure have direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

Intraparenchymal administration is the direct injection into the parenchyma (e.g., the brain parenchyma). Injection into the brain parenchyma allows for injection directly into brain regions affected by a disease or disorder while bypassing the blood brain barrier.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

In some embodiments of the methods described herein, the therapeutic composition may be delivered to the subject by way of systemic administration, e.g., intravenously, intramuscularly, or subcutaneously.

Intravenous (IV) injection is a method to directly inject into the bloodstream of a subject. The IV administration may be in the form of a bolus dose or by way of continuous infusion, or any other method tolerated by the therapeutic composition.

Intramuscular (IM) injection is injection into a muscle of a subject, such as the deltoid muscle or gluteal muscle. IM may allow for rapid absorption of the therapeutic composition.

Subcutaneous injection is injection into subcutaneous tissue. Absorption of compositions delivered subcutaneously may be slower than IV or IM injection, which may be beneficial for compositions requiring continuous absorption.

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. C9orf72 Knockdown

### Objective

This Example describes the results of a series of experiments undertaken to investigate the ability of siRNA molecules complementary to specific regions within a human C9orf72 mRNA transcript to effectuate reduced expression of the C9orf72 gene.

### Materials and Methods

SK-Mel-28 cells were actively transfected with C9orf72 siRNA at either 20 nM or 0.5 nM concentration. After 72 hours, cells were lysed, and mRNA levels of C9orf72 and a housekeeping gene (ATP5b) were assessed via reverse transcription-quantitative polymerase chain reaction (RT-qPCR), using standard reagents and Applied Biosystems TaqMan Assays. Results are presented as the percent residual C9orf72 mRNA relative to untreated control cells in the same assay (% untreated C9orf72 mRNA).

### Results

Cells were treated with an siRNA molecule of the disclosure having an antisense strand and a sense strand as shown in Tables 3 and 4, below. The knockdown efficiency of the siRNA molecule was measured as the residual mRNA expression as a percent of untreated at 20 nM and 0.5 nM. The knockdown results are reported in Table 3 (20 nM) and Table 4 (0.5 nM).

TABLE 3
C9orf72 knockdown with siRNA molecules
of the disclosure at 20 nM

| | | Residual |
|---|---|---|
| | | mRNA |
| | | expression |
| Sense | Antisense | (% of |
| SEQ ID | SEQ ID | untreated) |
| NO: | NO: | at 20 nM |
| 385 | 769 | 74.00 |
| 386 | 770 | 85.78 |
| 387 | 771 | 79.70 |
| 388 | 772 | 77.28 |
| 389 | 773 | 54.30 |
| 390 | 774 | 64.79 |
| 391 | 775 | 58.64 |
| 392 | 776 | 54.69 |
| 393 | 777 | 55.58 |
| 394 | 778 | 51.96 |
| 395 | 779 | 58.93 |
| 396 | 780 | 44.61 |
| 397 | 781 | 86.51 |
| 398 | 782 | 102.55 |
| 399 | 783 | 112.99 |
| 400 | 784 | 91.22 |
| 401 | 785 | 89.90 |
| 402 | 786 | 83.64 |
| 403 | 787 | 61.39 |
| 404 | 788 | 65.81 |
| 405 | 789 | 103.09 |
| 406 | 790 | 54.51 |
| 407 | 791 | 52.45 |
| 408 | 792 | 50.84 |
| 409 | 793 | 69.80 |
| 410 | 794 | 96.64 |
| 411 | 795 | 119.22 |
| 412 | 796 | 114.33 |
| 413 | 797 | 90.95 |
| 414 | 798 | 107.05 |
| 415 | 799 | 91.26 |
| 416 | 800 | 94.56 |
| 417 | 801 | 75.07 |
| 418 | 802 | 99.10 |
| 419 | 803 | 74.43 |
| 420 | 804 | 53.74 |
| 421 | 805 | 47.34 |
| 422 | 806 | 58.39 |
| 423 | 807 | 65.98 |
| 424 | 808 | 77.90 |
| 425 | 809 | 71.81 |
| 426 | 810 | 61.33 |
| 427 | 811 | 48.92 |
| 428 | 812 | 58.04 |
| 429 | 813 | 49.80 |
| 430 | 814 | 68.66 |
| 431 | 815 | 34.03 |
| 432 | 816 | 39.74 |
| 433 | 817 | 49.56 |
| 434 | 818 | 62.23 |
| 435 | 819 | 87.09 |
| 436 | 820 | 72.18 |
| 437 | 821 | 82.11 |
| 438 | 822 | 62.77 |
| 439 | 823 | 81.36 |
| 440 | 824 | 63.83 |
| 441 | 825 | 66.81 |
| 442 | 826 | 74.00 |
| 443 | 827 | 36.80 |
| 444 | 828 | 58.77 |
| 445 | 829 | 74.49 |
| 446 | 830 | 53.66 |
| 447 | 831 | 74.80 |
| 448 | 832 | 79.27 |
| 449 | 833 | 74.76 |
| 450 | 834 | 76.84 |
| 451 | 835 | 60.15 |
| 452 | 836 | 79.33 |
| 453 | 837 | 63.84 |
| 454 | 838 | 66.99 |
| 455 | 839 | 69.32 |
| 456 | 840 | 55.81 |
| 457 | 841 | 73.89 |
| 458 | 842 | 80.64 |
| 459 | 843 | 57.18 |
| 460 | 844 | 59.46 |
| 461 | 845 | 47.81 |
| 462 | 846 | 34.66 |
| 463 | 847 | 60.85 |
| 464 | 848 | 53.90 |
| 465 | 849 | 74.73 |
| 466 | 850 | 54.02 |
| 467 | 851 | 51.19 |
| 468 | 852 | 45.04 |
| 469 | 853 | 43.66 |
| 470 | 854 | 58.92 |
| 471 | 855 | 60.19 |
| 472 | 856 | 75.60 |
| 473 | 857 | 59.15 |
| 474 | 858 | 50.57 |
| 475 | 859 | 84.36 |
| 476 | 860 | 59.34 |
| 477 | 861 | 65.52 |
| 478 | 862 | 56.57 |
| 479 | 863 | 41.69 |
| 480 | 864 | 63.84 |
| 481 | 865 | 52.36 |
| 482 | 866 | 71.01 |
| 483 | 867 | 44.30 |
| 484 | 868 | 49.39 |
| 485 | 869 | 46.47 |
| 486 | 870 | 57.40 |
| 487 | 871 | 49.00 |
| 488 | 872 | 46.76 |
| 489 | 873 | 57.33 |
| 490 | 874 | 80.26 |
| 491 | 875 | 32.25 |
| 492 | 876 | 68.91 |
| 493 | 877 | 50.10 |
| 494 | 878 | 92.45 |
| 495 | 879 | 78.79 |
| 496 | 880 | 74.28 |
| 497 | 881 | 77.65 |
| 498 | 882 | 74.29 |
| 499 | 883 | 71.13 |
| 500 | 884 | 49.89 |
| 501 | 885 | 63.02 |
| 502 | 886 | 81.52 |
| 503 | 887 | 46.23 |
| 504 | 888 | 67.99 |
| 505 | 889 | 57.11 |
| 506 | 890 | 68.80 |
| 507 | 891 | 63.82 |
| 508 | 892 | 53.86 |
| 509 | 893 | 58.30 |
| 510 | 894 | 64.76 |
| 511 | 895 | 46.48 |
| 512 | 896 | 47.97 |
| 513 | 897 | 64.87 |
| 514 | 898 | 51.53 |
| 515 | 899 | 36.03 |
| 516 | 900 | 70.51 |
| 517 | 901 | 79.20 |
| 518 | 902 | 90.79 |
| 519 | 903 | 57.01 |
| 520 | 904 | 75.99 |
| 521 | 905 | 78.42 |
| 522 | 906 | 98.79 |
| 523 | 907 | 72.07 |
| 524 | 908 | 92.75 |
| 525 | 909 | 59.36 |
| 526 | 910 | 58.82 |
| 527 | 911 | 34.47 |
| 528 | 912 | 84.04 |
| 529 | 913 | 92.25 |
| 530 | 914 | 55.10 |
| 531 | 915 | 52.36 |
| 532 | 916 | 60.91 |
| 533 | 917 | 62.38 |
| 534 | 918 | 70.29 |
| 535 | 919 | 79.64 |
| 536 | 920 | 63.22 |
| 537 | 921 | 93.23 |
| 538 | 922 | 83.98 |
| 539 | 923 | 74.11 |
| 540 | 924 | 90.48 |
| 541 | 925 | 64.63 |
| 542 | 926 | 61.97 |
| 543 | 927 | 60.43 |
| 544 | 928 | 66.78 |
| 545 | 929 | 93.84 |
| 546 | 930 | 45.91 |
| 547 | 931 | 49.41 |
| 548 | 932 | 78.51 |
| 549 | 933 | 60.34 |
| 550 | 934 | 55.75 |
| 551 | 935 | 59.67 |
| 552 | 936 | 60.02 |
| 553 | 937 | 46.00 |
| 554 | 938 | 67.79 |
| 555 | 939 | 73.43 |
| 556 | 940 | 61.62 |
| 557 | 941 | 61.09 |
| 558 | 942 | 53.05 |
| 559 | 943 | 47.16 |
| 560 | 944 | 67.65 |
| 561 | 945 | 52.99 |
| 562 | 946 | 57.49 |
| 563 | 947 | 92.57 |
| 564 | 948 | 93.27 |
| 565 | 949 | 75.14 |
| 566 | 950 | 39.93 |
| 567 | 951 | 61.67 |
| 568 | 952 | 37.64 |
| 569 | 953 | 52.46 |
| 570 | 954 | 63.78 |
| 571 | 955 | 67.30 |
| 572 | 956 | 58.16 |
| 573 | 957 | 55.93 |
| 574 | 958 | 66.79 |
| 575 | 959 | 62.89 |
| 576 | 960 | 79.90 |

TABLE 4
C9orf72 knockdown with siRNA molecules
of the disclosure at 0.5 nM

| | | Residual |
|---|---|---|
| | | mRNA |
| | | expression |
| Sense | Antisense | (% of |
| SEQ ID | SEQ ID | untreated) |
| NO: | NO: | at 0.5 nM |
| 385 | 769 | 84.18 |
| 386 | 770 | 97.05 |
| 387 | 771 | 81.22 |
| 388 | 772 | 97.76 |
| 389 | 773 | 79.78 |
| 390 | 774 | 51.41 |
| 391 | 775 | 79.03 |
| 392 | 776 | 58.50 |
| 393 | 777 | 61.25 |
| 394 | 778 | 65.12 |
| 395 | 779 | 86.17 |
| 396 | 780 | 84.40 |
| 397 | 781 | 94.73 |
| 398 | 782 | 92.59 |
| 399 | 783 | 92.94 |
| 400 | 784 | 99.21 |
| 401 | 785 | 92.58 |
| 402 | 786 | 92.91 |
| 403 | 787 | 93.13 |
| 404 | 788 | 93.43 |
| 405 | 789 | 99.32 |
| 406 | 790 | 87.63 |
| 407 | 791 | 101.10 |
| 408 | 792 | 98.80 |
| 409 | 793 | 74.44 |
| 410 | 794 | 98.27 |
| 411 | 795 | 101.08 |
| 412 | 796 | 100.91 |
| 413 | 797 | 93.69 |
| 414 | 798 | 102.04 |
| 415 | 799 | 95.92 |
| 416 | 800 | 95.63 |
| 417 | 801 | 99.42 |
| 418 | 802 | 105.08 |
| 419 | 803 | 90.83 |
| 420 | 804 | 85.58 |
| 421 | 805 | 69.67 |
| 422 | 806 | 66.74 |
| 423 | 807 | 84.63 |
| 424 | 808 | 71.51 |
| 425 | 809 | 84.20 |
| 426 | 810 | 79.29 |
| 427 | 811 | 71.81 |
| 428 | 812 | 66.27 |
| 429 | 813 | 88.06 |
| 430 | 814 | 104.23 |
| 431 | 815 | 73.08 |
| 432 | 816 | 66.69 |
| 433 | 817 | 72.79 |
| 434 | 818 | 81.19 |
| 435 | 819 | 98.19 |
| 436 | 820 | 101.08 |
| 437 | 821 | 95.62 |
| 438 | 822 | 86.32 |
| 439 | 823 | 96.12 |
| 440 | 824 | 85.32 |
| 441 | 825 | 94.05 |
| 442 | 826 | 93.73 |
| 443 | 827 | 82.68 |
| 444 | 828 | 88.17 |
| 445 | 829 | 83.81 |
| 446 | 830 | 84.38 |
| 447 | 831 | 94.46 |
| 448 | 832 | 88.26 |
| 449 | 833 | 87.97 |
| 450 | 834 | 84.91 |
| 451 | 835 | 88.73 |
| 452 | 836 | 93.46 |
| 453 | 837 | 96.57 |
| 454 | 838 | 96.64 |
| 455 | 839 | 87.89 |
| 456 | 840 | 85.89 |
| 457 | 841 | 77.76 |
| 458 | 842 | 95.41 |
| 459 | 843 | 69.57 |
| 460 | 844 | 71.21 |
| 461 | 845 | 64.29 |
| 462 | 846 | 70.72 |
| 463 | 847 | 94.36 |
| 464 | 848 | 81.27 |
| 465 | 849 | 84.43 |
| 466 | 850 | 94.31 |
| 467 | 851 | 91.42 |
| 468 | 852 | 94.65 |
| 469 | 853 | 58.63 |
| 470 | 854 | 68.50 |
| 471 | 855 | 69.36 |
| 472 | 856 | 89.47 |
| 473 | 857 | 88.74 |
| 474 | 858 | 78.16 |
| 475 | 859 | 70.96 |
| 476 | 860 | 94.34 |
| 477 | 861 | 78.84 |
| 478 | 862 | 93.03 |
| 479 | 863 | 91.53 |
| 480 | 864 | 95.85 |
| 481 | 865 | 52.13 |
| 482 | 866 | 82.47 |
| 483 | 867 | 62.75 |
| 484 | 868 | 86.17 |
| 485 | 869 | 50.62 |
| 486 | 870 | 69.02 |
| 487 | 871 | 55.35 |
| 488 | 872 | 61.01 |
| 489 | 873 | 83.95 |
| 490 | 874 | 95.17 |
| 491 | 875 | 77.58 |
| 492 | 876 | 86.29 |
| 493 | 877 | 67.01 |
| 494 | 878 | 80.27 |
| 495 | 879 | 111.22 |
| 496 | 880 | 85.77 |
| 497 | 881 | 87.74 |
| 498 | 882 | 58.48 |
| 499 | 883 | 94.27 |
| 500 | 884 | 85.55 |
| 501 | 885 | 97.34 |
| 502 | 886 | 107.06 |
| 503 | 887 | 104.85 |
| 504 | 888 | 86.50 |
| 505 | 889 | 78.24 |
| 506 | 890 | 111.98 |
| 507 | 891 | 117.07 |
| 508 | 892 | 96.64 |
| 509 | 893 | 80.66 |
| 510 | 894 | 95.83 |
| 511 | 895 | 80.29 |
| 512 | 896 | 72.07 |
| 513 | 897 | 99.75 |
| 514 | 898 | 89.84 |
| 515 | 899 | 90.49 |
| 516 | 900 | 89.41 |
| 517 | 901 | 134.33 |
| 518 | 902 | 126.60 |
| 519 | 903 | 109.67 |
| 520 | 904 | 134.21 |
| 521 | 905 | 99.58 |
| 522 | 906 | 83.35 |
| 523 | 907 | 102.65 |
| 524 | 908 | 99.01 |
| 525 | 909 | 75.79 |
| 526 | 910 | 98.36 |
| 527 | 911 | 96.92 |
| 528 | 912 | 89.97 |
| 529 | 913 | 126.77 |
| 530 | 914 | 94.63 |
| 531 | 915 | 79.73 |
| 532 | 916 | 64.65 |
| 533 | 917 | 107.40 |
| 534 | 918 | 89.47 |
| 535 | 919 | 95.48 |
| 536 | 920 | 100.38 |
| 537 | 921 | 110.71 |
| 538 | 922 | 108.97 |
| 539 | 923 | 82.87 |
| 540 | 924 | 84.50 |
| 541 | 925 | 80.19 |
| 542 | 926 | 124.01 |
| 543 | 927 | 80.66 |
| 544 | 928 | 97.55 |
| 545 | 929 | 94.59 |
| 546 | 930 | 49.41 |
| 547 | 931 | 76.37 |
| 548 | 932 | 99.19 |
| 549 | 933 | 66.92 |
| 550 | 934 | 76.66 |
| 551 | 935 | 90.08 |
| 552 | 936 | 89.27 |
| 553 | 937 | 44.62 |
| 554 | 938 | 114.96 |
| 555 | 939 | 106.06 |
| 556 | 940 | 85.79 |
| 557 | 941 | 60.33 |
| 558 | 942 | 75.62 |
| 559 | 943 | 70.66 |
| 560 | 944 | 69.97 |
| 561 | 945 | 96.74 |
| 562 | 946 | 81.67 |
| 563 | 947 | 92.52 |
| 564 | 948 | 109.02 |
| 565 | 949 | 59.35 |
| 566 | 950 | 58.60 |
| 567 | 951 | 71.91 |
| 568 | 952 | 76.83 |
| 569 | 953 | 58.74 |
| 570 | 954 | 50.59 |
| 571 | 955 | 67.64 |
| 572 | 956 | 49.70 |
| 573 | 957 | 52.16 |
| 574 | 958 | 68.24 |
| 575 | 959 | 95.02 |
| 576 | 960 | 105.03 |

### Example 2. Generating C9orf72-Targeting siRNA Molecules

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or modified nucleotides can be easily prepared. Specific examples of siRNA molecules, with the nucleotide sequence of the sense and antisense strand, as well as the C9orf72 mRNA target sequence, are shown in Table 1, above. It is appreciated that one of skill in the art could anneal the antisense (AS) strand to the corresponding sense(S) strand to yield a ds-siRNA molecule. Alternatively, one of skill in the art could derive a ss-siRNA molecule using antisense strand only.

### Example 3. Optimizing C9orf72-Targeting siRNA Molecules

It is contemplated that for any small interfering RNA (siRNA) agent disclosed herein, modifications to the siRNA may further optimize the molecule's efficacy or biophysical properties (e.g., increasing serum stability or circulating half-life, increasing thermal stability, enhancing transmembrane delivery, and/or targeting to a particular location or cell type). Such optimization could be achieved by systematically either adding or removing linked nucleosides to generate longer or shorter sequences. Further siRNA optimization could include the incorporation of, for example, one or more alternative nucleosides, alternative 2′ sugar moieties, and/or alternative internucleoside linkages. Further still, such optimized siRNA molecules may include the introduction of hydrophobic and/or stabilizing moieties at the 5′ and/or 3′ ends.

siRNA Optimization with Alternative Nucleosides

Optimization of the siRNA molecules of the disclosure may include one or more of the following nucleoside modifications: 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine, and/or 3-deazaguanine and 3-deazaadenine. The siRNA molecules may also include nucleobases in which the purine or pyrimidine base is replaced with other heterocycles, for example 7-deaza-adenine, 7-deazaguanosine, 2-aminopyridine, and/or 2-pyridone. Further optimization of the siRNA molecules of the disclosure may include nucleobases disclosed in U.S. Pat. No. 3,687,808; Kroschwitz, J. I., ed. The Concise Encyclopedia of Polymer Science and Engineering, New York, John Wiley & Sons, 1990, pp. 858-859; Englisch et al., Angewandte Chemie, International Edition 30:613, 1991; and Sanghvi, Y. S., Chapter 16, Antisense Research and Applications, CRC Press, Gait, M. J. ed., 1993, pp. 289-302.

SiRNA Optimization with Alternative Sugar Modifications

Optimization of the siRNA molecules of the disclosure may include one or more of the following 2′ sugar modifications: 2′-O-methyl (2′-O-Me), 2′-methoxyethoxy (2′-O—CH₂CH₂OCH₃, also known as 2′-O-(2-methoxyethyl) or 2′-MOE), 2′-dimethylaminooxyethoxy, i.e., a O(CH₂)₂ON(CH₃)₂ group, also known as 2′-DMAOE, and/or 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other possible 2′-modifications that can optimize the siRNA molecules of the disclosure include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

siRNA Optimization with Alternative Internucleoside Linkages

Optimization of the siRNA molecules of the disclosure may include one or more of the following internucleoside modifications: phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

SIRNA Optimization with Hydrophobic Moieties

Optimization of the siRNA molecules of the disclosure may include hydrophobic moieties covalently attached to the 5′ end or the 3′ end. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

SIRNA Optimization with Stabilizing Moieties

Optimization of the siRNA molecules of the disclosure may include a 5′-phosphorous stabilizing moiety that protects the siRNA molecules from degradation. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each siRNA strand may independently and optionally employ any suitable 5′-phosphorus stabilizing moiety. Non-limiting examples of 5′ stabilizing moieties suitable for use with the siRNA molecules of the disclosure may include those demonstrated by Formulas IX-XVI above.

siRNA Optimization with Branched siRNA

Optimization of the siRNA molecules of the disclosure may include the incorporation of branching patterns, such as, for example, di-branched, tri-branched, or tetra-branched siRNAs connected by way of a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2, above.

The siRNA composition of the disclosure may be optimized to be in the form of: di-branched siRNA molecules, as represented by any one of Formulas XVII-XIX; tri-branched siRNA molecules, as represented by any one of Formulas XX-XXIII; and/or tetra-branched siRNA molecules, as represented by any one of Formulas XXIV-XXVIII, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

### Example 4. Preparation and Administrating C9orf72-Targeting siRNA Molecules

The siRNA molecules in the present disclosure may be formulated into a pharmaceutical composition for administration to a subject in a biologically compatible form suitable for administration in vivo. For example, the siRNA molecules of the disclosure may be administered in a suitable diluent, carrier, or excipient, and may further contain a preservative, e.g., to prevent the growth of microorganisms. Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

The method of the disclosure contemplates any route of administration to the subject that is tolerated by the siRNA compositions of the disclosure. Non-limiting examples of siRNA injections into the CNS include intrathecally, intracerebroventricularly, intrastriatally, intraparenchymally, or intra-cisterna magna injection by catheterization. Examples of systemic administration include intravenous, intramuscular, and subcutaneous injection. A physician having ordinary skill in the art can readily determine an effective route of administration.

### Example 5. Methods for the Treatment of Amyotrophic Lateral Sclerosis or Frontotemporal Dementia Using C9orf72-Targeting siRNA Molecules

A subject in need of treatment of amyotrophic lateral sclerosis (ALS) or frontotemporal dementia (FTD), is treated with a dosage of the siRNA molecule or siRNA composition of the disclosure, formulated as a salt, at frequency determined by a practitioner. A physician having ordinary skill in the art can readily determine an effective amount of the siRNA molecule for administration to a mammalian subject (e.g., a human) in need thereof. For example, a physician could start prescribing doses of one of the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until a minimum dose that produces a therapeutic effect (e.g., a reduction in expression of C9orf72 mRNA or suitable biomarker or a reduction of ALS- or FTD-associated phenotypes) is achieved. In general, a suitable daily dose of one of one of the siRNA molecules of the disclosure will be an amount which is the lowest dose effective to produce a therapeutic effect. The ss- or ds-siRNA molecules of the disclosure may be administered by injection, e.g., intrathecally, intracerebroventricularly, intrastriatally, intraparenchymally, intravenously, intramuscularly, subcutaneously, or by intra-cisterna magna injection via catheterization. A daily dose of a therapeutic composition of one of the siRNA molecules of the disclosure may be administered as a single dose or as two, three, four, five, six or more doses administered separately at appropriate intervals throughout the day, week, month, or year, optionally, in unit dosage forms. While it is possible for any of the SIRNA molecules of the disclosure to be administered alone, it may also be administered as a pharmaceutical formulation in combination with excipients, carriers, and optionally, additional therapeutic agents. Dosage and frequency are determined based on the subject's height, weight, age, sex, and other disorders.

The siRNA molecule(s) of the disclosure is selected by the practitioner for compatibility with the subject. Single- or double-stranded siRNA molecules (e.g., non-branched siRNA, di-branched SIRNA, tri-branched siRNA, tetra-branched siRNA) are available for selection. The siRNA molecule chosen has an antisense strand and may have a sense strand with a sequence and RNA modifications (e.g., natural and non-natural internucleoside linkages, modified sugars, 5′-phosphorus stabilizing moieties, hydrophobic moieties, and/or branching structures) best suited to the patient.

The siRNA molecule is delivered by the route best suited the patient (e.g., intrathecally, intracerebroventricularly, intrastriatally, intraparenchymally, intravenously, intramuscularly, subcutaneously, or by intra-cisterna magna injection via catheterization) and condition at a rate tolerable to the patient until the subject has reached a maximum tolerated dose, or until symptoms are ameliorated satisfactorily.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A small interfering RNA (siRNA) molecule comprising an antisense strand and sense strand having complementarity to the antisense strand, wherein the antisense strand has complementarity sufficient to hybridize to a region within a chromosome 9 open reading frame 72 (C9orf72) mRNA transcript having the nucleic acid sequence of any one of SEQ ID NOs: 1-384.

2. The siRNA molecule of claim 1, wherein the antisense strand has at least 70% complementarity to a region of 19, 20, 21, or more contiguous nucleobases within the C9orf72 mRNA transcript having the nucleic acid sequence of any one of SEQ ID NOs: 1-384, optionally wherein the antisense strand has at least 70% complementarity to the C9orf72 mRNA transcript having the nucleic acid sequence of any one of SEQ ID NOs: 1-384.

3. The siRNA molecule of claim 2, wherein the antisense strand has at least 75% complementarity to a region of 21 contiguous nucleobases within the C9orf72 mRNA transcript having the nucleic acid sequence of any one of SEQ ID NOs: 1-384, optionally wherein the antisense strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the C9orf72 mRNA transcript having the nucleic acid sequence of any one of SEQ ID Nos: 1-384.

4. The siRNA molecule of any one of claims 1-3, wherein the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the C9orf72 RNA transcript having the nucleic acid sequence of any one of SEQ ID NOS: 1-384.

5. The siRNA molecule of claim 4, wherein the antisense strand comprises from 10 to 30 contiguous nucleotides that are fully complementary to a contiguous polynucleotide segment of equal length within the region of the C9orf72 RNA transcript having the nucleic acid sequence of any one of SEQ ID NOS: 1-384.

6. The siRNA molecule of claim 5, wherein the antisense strand comprises from 12 to 30 contiguous nucleotides that are fully complementary to a contiguous polynucleotide segment of equal length within the region of the C9orf72 RNA transcript having the nucleic acid sequence of any one of SEQ ID NOs: 1-384.

7. The siRNA molecule of claim 6, wherein the antisense strand comprises from 15 to 30 contiguous nucleotides that are fully complementary to a contiguous polynucleotide segment of equal length within the region of the C9orf72 RNA transcript having the nucleic acid sequence of any one of SEQ ID NOS: 1-384.

8. The siRNA molecule of claim 7, wherein the antisense strand comprises from 18 to 30 contiguous nucleotides that are fully complementary to a contiguous polynucleotide segment of equal length within the region of the C9orf72 RNA transcript having the nucleic acid sequence of any one of SEQ ID NOs: 1-384.

9. The siRNA molecule of claim 8, wherein the antisense strand comprises from 18 to 25 contiguous nucleotides that are fully complementary to a contiguous polynucleotide segment of equal length within the region of the C9orf72 RNA transcript having the nucleic acid sequence of any one of SEQ ID NOs: 1-384.

10. The siRNA molecule of any one of claim 9, wherein the antisense strand comprises from 18 to 21 contiguous nucleotides that are fully complementary to a contiguous polynucleotide segment of equal length within the region of the C9orf72 RNA transcript having the nucleic acid sequence of any one of SEQ ID NOs: 1-384.

11. The siRNA molecule of claim 10, wherein the antisense strand comprises 21 contiguous nucleotides that are fully complementary to a contiguous polynucleotide segment of equal length within the region of the C9orf72 RNA transcript having the nucleic acid sequence of any one of SEQ ID NOs: 1-384.

12. The siRNA molecule of any one of claims 1-11, wherein the antisense strand comprises 9 or fewer nucleotide mismatches relative to a region of 21 contiguous nucleobases of the C9orf72 RNA transcript having the nucleic acid sequence of any one of SEQ ID NOS: 1-384, optionally wherein the antisense strand comprises 8 or fewer, 7 or fewer, 6 or fewer, 5 or fewer, 4 or fewer, 3 or fewer, 2 or fewer, or only 1 mismatch relative to the region of the C9orf72 RNA transcript having the nucleic acid sequence of any one of SEQ ID NOS: 1-384.

13. The siRNA molecule of any one of claims 1-12, wherein the region of the C9orf72 RNA transcript has the nucleic acid sequence of any one of SEQ ID NOS: 1-192.

14. The siRNA molecule of any one of claims 1-12, wherein the region of the C9orf72 RNA transcript has the nucleic acid sequence of any one of SEQ ID NOs: 193-384.

15. The siRNA molecule of any one of claims 1-14, wherein the antisense strand has a nucleic acid sequence that is at least 85% identical to the nucleic acid sequence of any one of SEQ ID NOs: 769-1152.

16. The siRNA molecule of claim 15, wherein the antisense strand has a nucleic acid sequence that is at least 90% identical to the nucleic acid sequence of any one of SEQ ID NOs: 769-1152.

17. The siRNA molecule of claim 16, wherein the antisense strand has a nucleic acid sequence that is at least 95% identical to the nucleic acid sequence of SEQ ID NOs: 769-1152, optionally wherein the antisense strand has a nucleic acid sequence that is at least 96%, 97%, 98%, or 99% identical to the nucleic acid sequence of any one of SEQ ID NOS: 769-1152.

18. The siRNA molecule of claim 17, wherein the antisense strand has the nucleic acid sequence of any one of SEQ ID NOS: 769-1152.

19. The siRNA molecule of any one of claims 15-18, wherein the nucleic acid sequence is any one of SEQ ID NOs: 769-960.

20. The siRNA molecule of any one of claims 15-18, wherein the nucleic acid sequence is any one of SEQ ID NOS: 961-1152.

21. The siRNA molecule of any one of claims 1-20, wherein the sense strand has a nucleic acid sequence that is at least 85% identical to the nucleic acid sequence of any one of SEQ ID NOS: 385-768.

22. The siRNA molecule of claim 21, wherein the sense strand has a nucleic acid sequence that is at least 90% identical to the nucleic acid sequence of any one of SEQ ID NOs: 385-768.

23. The siRNA molecule of claim 22, wherein the sense strand has a nucleic acid sequence that is at least 95% identical to the nucleic acid sequence of SEQ ID NOS: 385-768, optionally wherein the sense strand has a nucleic acid sequence that is at least 96%, 97%, 98%, or 99% identical to the nucleic acid sequence of any one of SEQ ID NOS: 385-768.

24. The siRNA molecule of claim 23, wherein the sense strand has the nucleic acid sequence of any one of SEQ ID NOs: 385-768.

25. The siRNA molecule of any one of claims 21-24, wherein the nucleic acid sequence is any one of SEQ ID NOs: 385-576.

26. The siRNA molecule of any one of claims 21-24, wherein the nucleic acid sequence is any one of SEQ ID NOs: 577-768.

27. The siRNA molecule of any one of claims 1-26, wherein the antisense strand comprises a structure represented by Formula I, wherein Formula I is, in the 5′-to-3′ direction: 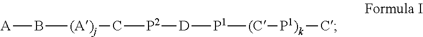
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

28. The siRNA molecule of claim 27, wherein the antisense strand comprises a structure represented by Formula A1, wherein Formula A1 is, in the 5′-to-3′ direction: 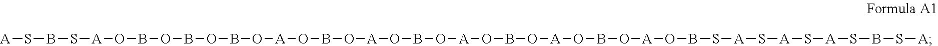
wherein A represents a 2′-O-Me ribonucleoside, B represents a 2′-F ribonucleoside, O represents a phosphodiester internucleoside linkage, and S represents a phosphorothioate internucleoside linkage.

29. The siRNA molecule of any one of claims 1-26, wherein the antisense strand comprises a structure represented by Formula II, wherein Formula II is, in the 5′-to-3′ direction: 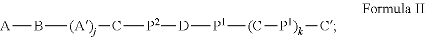
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

30. The siRNA molecule of claim 29, wherein the antisense strand comprises a structure represented by Formula A2, wherein Formula A2 is, in the 5′-to-3′ direction: 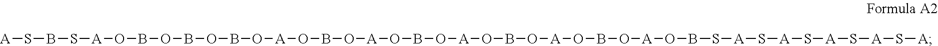
wherein A represents a 2′-O-Me ribonucleoside, B represents a 2′-F ribonucleoside, O represents a phosphodiester internucleoside linkage, and S represents a phosphorothioate internucleoside linkage.

31. The siRNA molecule of any one of claims 1-30, wherein the sense strand comprises a structure represented by Formula III, wherein Formula III is, in the 5′-to-3′ direction: 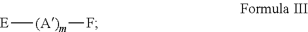
wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7.

32. The siRNA molecule of claim 31, wherein the sense strand comprises a structure represented by Formula S1, wherein Formula S1 is, in the 5′-to-3′ direction: 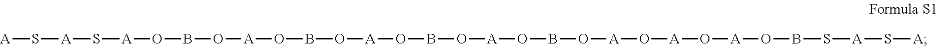
wherein A represents a 2′-O-Me ribonucleoside, B represents a 2′-F ribonucleoside, O represents a phosphodiester internucleoside linkage, and S represents a phosphorothioate internucleoside linkage.

33. The siRNA molecule of claim 31, wherein the sense strand comprises a structure represented by Formula S2, wherein Formula S2 is, in the 5′-to-3′ direction: 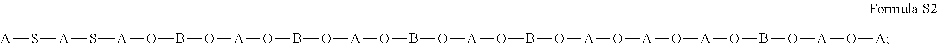
wherein A represents a 2′-O-Me ribonucleoside, B represents a 2′-F ribonucleoside, O represents a phosphodiester internucleoside linkage, and S represents a phosphorothioate internucleoside linkage.

34. The siRNA molecule of claim 31, wherein the sense strand comprises a structure represented by Formula S3, wherein Formula S3 is, in the 5′-to-3′ direction: 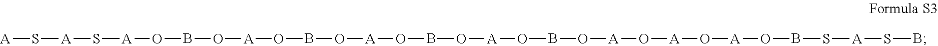
wherein A represents a 2′-O-Me ribonucleoside, B represents a 2′-F ribonucleoside, O represents a phosphodiester internucleoside linkage, and S represents a phosphorothioate internucleoside linkage.

35. The siRNA molecule of claim 31, wherein the sense strand comprises a structure represented by Formula S4, wherein Formula S4 is, in the 5′-to-3′ direction: 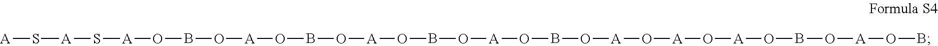
wherein A represents a 2′-O-Me ribonucleoside, B represents a 2′-F ribonucleoside, O represents a phosphodiester internucleoside linkage, and S represents a phosphorothioate internucleoside linkage.

36. The siRNA molecule of any one of claims 1-26 and 31-35, wherein the antisense strand comprises a structure represented by Formula IV, wherein Formula IV is, in the 5′-to-3′ direction: 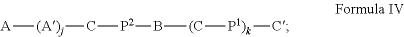
wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

37. The siRNA molecule of claim 36, wherein the antisense strand comprises a structure represented by Formula A3, wherein Formula A3 is, in the 5′-to-3′ direction: 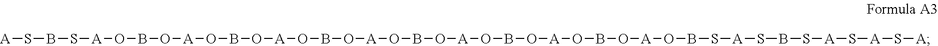
wherein A represents a 2′-O-Me ribonucleoside, B represents a 2′-F ribonucleoside, O represents a phosphodiester internucleoside linkage, and S represents a phosphorothioate internucleoside linkage.

38. The siRNA molecule of any one of claims 1-30, 36, and 37, wherein the sense strand comprises a structure represented by Formula V, wherein Formula V is, in the 5′-to-3′ direction: 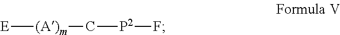
wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7.

39. The siRNA molecule of claim 38, wherein the sense strand comprises a structure represented by Formula S5, wherein Formula S5 is, in the 5′-to-3′ direction: 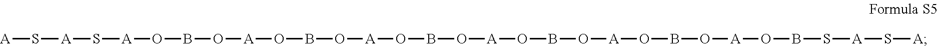
wherein A represents a 2′-O-Me ribonucleoside, B represents a 2′-F ribonucleoside, O represents a phosphodiester internucleoside linkage, and S represents a phosphorothioate internucleoside linkage.

40. The siRNA molecule of claim 38, wherein the sense strand comprises a structure represented by Formula S6, wherein Formula S6 is, in the 5′-to-3′ direction: 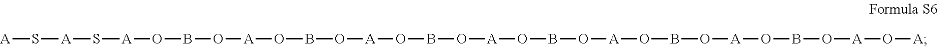
wherein A represents a 2′-O-Me ribonucleoside, B represents a 2′-F ribonucleoside, O represents a phosphodiester internucleoside linkage, and S represents a phosphorothioate internucleoside linkage.

41. The siRNA molecule of claim 38, wherein the sense strand comprises a structure represented by Formula S7, wherein Formula S7 is, in the 5′-to-3′ direction: 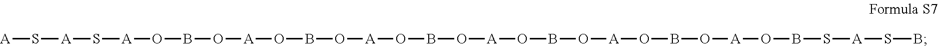
wherein A represents a 2′-O-Me ribonucleoside, B represents a 2′-F ribonucleoside, O represents a phosphodiester internucleoside linkage, and S represents a phosphorothioate internucleoside linkage.

42. The siRNA molecule of claim 38, wherein the sense strand comprises a structure represented by Formula S8, wherein Formula S8 is, in the 5′-to-3′ direction: 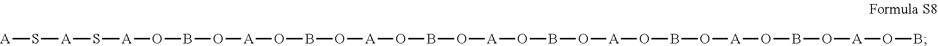
wherein A represents a 2′-O-Me ribonucleoside, B represents a 2′-F ribonucleoside, O represents a phosphodiester internucleoside linkage, and S represents a phosphorothioate internucleoside linkage.

43. The siRNA molecule of any one of claims 1-26, 31-35 and 38-42, wherein the antisense strand comprises a structure represented by Formula VI, wherein Formula VI is, in the 5′-to-3′ direction: 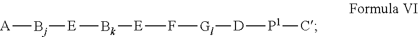
wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

44. The siRNA molecule of claim 43, wherein the antisense strand comprises a structure represented by Formula A4, wherein Formula A4 is, in the 5′-to-3′ direction: 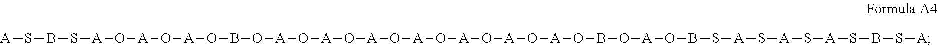
wherein A represents a 2′-O-Me ribonucleoside, B represents a 2′-F ribonucleoside, O represents a phosphodiester internucleoside linkage, and S represents a phosphorothioate internucleoside linkage.

45. The siRNA molecule of any one of claims 1-30, 36, 37, 43, and 44, wherein the sense strand comprises a structure represented by Formula VII, wherein Formula VII is, in the 5′-to-3′ direction: 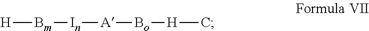
wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7.

46. The siRNA molecule of claim 45, wherein the sense strand comprises a structure represented by Formula S9, wherein Formula S9 is, in the 5′-to-3′ direction: 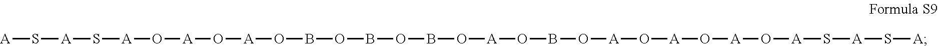
wherein A represents a 2′-O-Me ribonucleoside, B represents a 2′-F ribonucleoside, O represents a phosphodiester internucleoside linkage, and S represents a phosphorothioate internucleoside linkage.

47. The siRNA molecule of any one of claims 1-46, wherein the antisense strand further comprises a 5′ phosphorus stabilizing moiety at the 5′ end of the antisense strand.

48. The siRNA molecule of any one of claims 1-47, wherein the sense strand further comprises a 5′ phosphorus stabilizing moiety at the 5′ end of the sense strand.

49. The siRNA molecule of claim 47 or 48, wherein each 5′ phosphorus stabilizing moiety is, independently, represented by any one of Formulas IX-XVI: 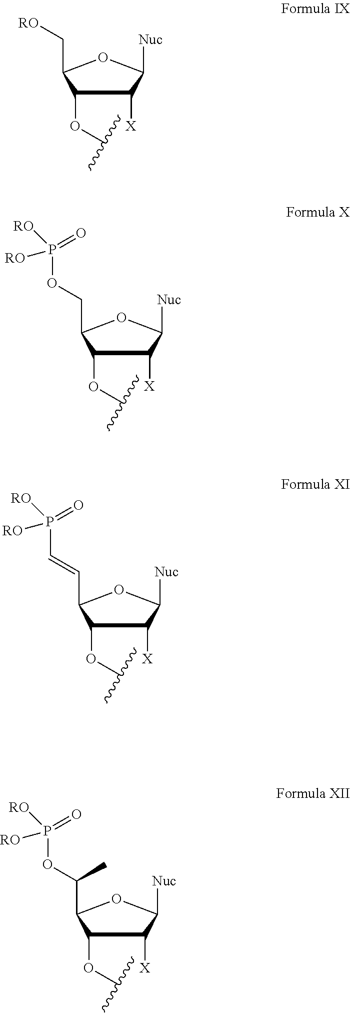 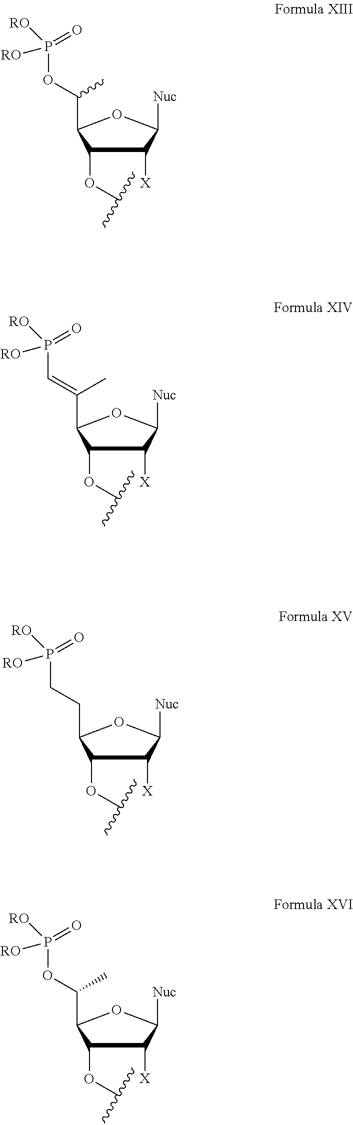
wherein Nuc represents a nucleobase, optionally wherein the nucleobase is selected from the group consisting of adenine, uracil, guanine, thymine, and cytosine, and R represents an optionally substituted alkyl, optionally substituted alkenyl, optionally substituted alkynyl, phenyl, benzyl, a cation, or hydrogen.

50. The siRNA molecule of claim 49, wherein the nucleobase is an adenine, uracil, guanine, thymine, or cytosine.

51. The siRNA molecule of any one of claims 47-50, wherein the 5′ phosphorus stabilizing moiety is (E)-vinylphosphonate represented by Formula XI.

52. The siRNA molecule of any one of claims 1-51, wherein the siRNA molecule further comprises a hydrophobic moiety at the 5′ or the 3′ end of the siRNA molecule.

53. The siRNA molecule of claim 52, wherein the hydrophobic moiety is selected from a group consisting of cholesterol, vitamin D, or tocopherol.

54. The siRNA molecule of any one of claims 1-53, wherein the length of the sense strand is between 10 and 30 nucleotides.

55. The siRNA molecule of claim 54, wherein the length of the sense strand is between 10 and 25 nucleotides.

56. The siRNA molecule of claim 55, wherein the length of the sense strand is between 12 and 25 nucleotides.

57. The siRNA molecule of claim 56, wherein the length of the sense strand is between 12 and 20 nucleotides.

58. The siRNA molecule of claim 57, wherein the length of the sense strand is between 12 and 19 nucleotides.

59. The siRNA molecule of claim 58, wherein the length of the sense strand is 15 nucleotides.

60. The siRNA molecule of claim 58, wherein the length of the sense strand is 16 nucleotides.

61. The siRNA molecule of claim 58, wherein the length of the sense strand is 18 nucleotides.

62. The siRNA molecule of any one of claims 1-61, wherein the length of the antisense strand is between 10 and 30 nucleotides.

63. The siRNA molecule of claim 62, wherein the length of the antisense strand is between 12 and 30 nucleotides.

64. The siRNA molecule of claim 63, wherein the length of the antisense strand is between 15 and 30 nucleotides.

65. The siRNA molecule of claim 64, wherein the length of the antisense strand is between 18 and 30 nucleotides.

66. The siRNA molecule of claim 65, wherein the length of the antisense strand is between 18 and 25 nucleotides.

67. The siRNA molecule of claim 66, wherein the length of the antisense strand is between 18 and 21 nucleotides.

68. The siRNA molecule of claim 67, wherein the length of the antisense strand is 18 nucleotides.

69. The siRNA molecule of claim 67, wherein the length of the antisense strand is 20 nucleotides.

70. The siRNA molecule of claim 67, wherein the length of the antisense strand is 21 nucleotides.

71. The siRNA molecule of any one of claims 1-70, wherein the siRNA molecule is a branched siRNA molecule.

72. The siRNA molecule of claim 71, wherein the branched siRNA molecule is di-branched, tri-branched, or tetra-branched.

73. The siRNA molecule of claim 72, wherein the siRNA molecule is a di-branched siRNA molecule, optionally wherein the di-branched siRNA molecule is represented by any one of Formulas XVII-XIX: 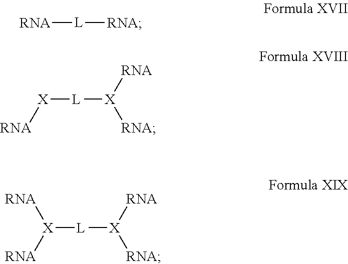
wherein each RNA is, independently, an siRNA molecule, L is a linker, and each X, independently, represents a branch point moiety.

74. The siRNA molecule of claim 72, wherein the siRNA molecule is a tri-branched siRNA molecule, optionally wherein the tri-branched siRNA molecule is represented by any one of Formulas XX-XXIII: 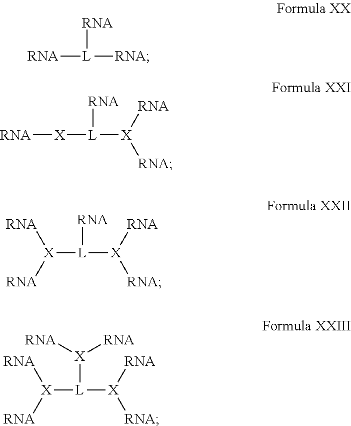
wherein each RNA is, independently, an siRNA molecule, L is a linker, and each X, independently, represents a branch point moiety.

75. The siRNA molecule of claim 72, wherein the siRNA molecule is a tetra-branched siRNA molecule, optionally wherein the tetra-branched siRNA molecule is represented by any one of Formulas XXIV-XXVIII: 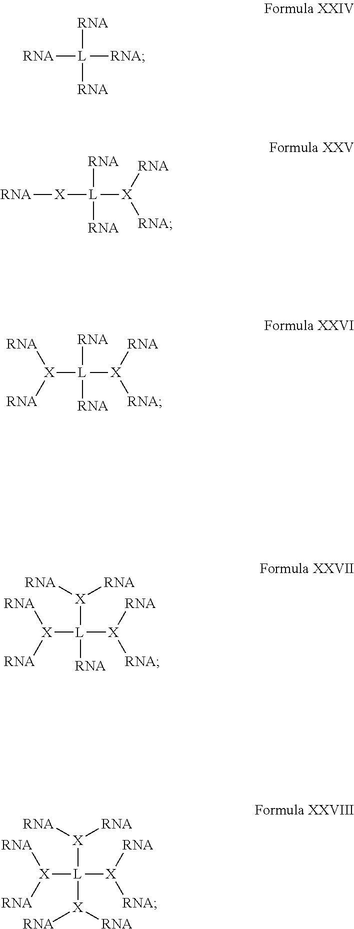
wherein each RNA is, independently, an siRNA molecule, L is a linker, and each X, independently, represents a branch point moiety.

76. The siRNA molecule of any one of claims 73-75, wherein the linker is selected from a group consisting of one or more contiguous subunits of an ethylene glycol, alkyl, carbohydrate, block copolymer, peptide, RNA, and DNA.

77. The siRNA molecule of claim 76, wherein the one or more contiguous subunits is 2 to 20 contiguous subunits.

78. A pharmaceutical composition comprising the siRNA molecule of any one of claims 1-77 and a pharmaceutically acceptable excipient, carrier, or diluent.

79. A method of delivering an siRNA molecule to a subject diagnosed as amyotrophic lateral sclerosis (ALS), the method comprising administering a therapeutically effective amount of the siRNA molecule of any one of claims 1-77 or the pharmaceutical composition of claim 78 to the subject.

80. A method of delivering an siRNA molecule to a subject diagnosed as frontotemporal dementia (FTD), the method comprising administering a therapeutically effective amount of the siRNA molecule of any one of claims 1-77 or the pharmaceutical composition of claim 78 to the subject.

81. A method of treating ALS in a subject in need thereof, the method comprising administering a therapeutically effective amount of the siRNA molecule of any one of claims 1-77 or the pharmaceutical composition of claim 78 to the subject.

82. A method of treating FTD in a subject in need thereof, the method comprising administering a therapeutically effective amount of the siRNA molecule of any one of claims 1-77 or the pharmaceutical composition of claim 78 to the subject.

83. A method of reducing C9orf72 expression in a subject in need thereof, the method comprising administering a therapeutically effective amount of the siRNA molecule of any one of claims 1-77 or the pharmaceutical composition of claim 78 to the subject.

84. The method of any one of claims 79-83, wherein the siRNA molecule or the pharmaceutical composition is administered to the subject by way of intracerebroventricular, intrastriatal, intraparenchymal, or intrathecal injection.

85. The method of any one of claims 79-83, wherein the siRNA molecule or the pharmaceutical composition is administered to the subject by way of intravenous, intramuscular, or subcutaneous injection.

86. The method of any one of claims 79-85, wherein the subject is a human.

87. A kit comprising the siRNA molecule of any one of claims 1-77, or the pharmaceutical composition of claim 78, and a package insert, wherein the package insert instructs a user of the kit to perform the method of any one of claims 79-86.
